(19) United States
(12) Patent Application Publication
(10) Pub. No.: US 2015/0118187 A1
(43) Pub. Date: Apr. 30, 2015

(54) MICRO-ORGANS PROVIDING SUSTAINED DELIVERY OF A THERAPEUTIC POLYPEPTIDE AND METHODS OF USE THEREOF

(71) Applicant: MEDGENICS MEDICAL ISRAEL LTD., Misgav (IL)

(72) Inventors: Nir Shapir, DM Misgav (IL); Reem Miari, Sakhnin (IL); Baruch S. Stern, Haifa (IL); Shany Blum, Haifa (IL); Avi Rimler, Ness Ziona (IL)

(73) Assignee: MEDGENICS MEDICAL ISRAEL LTD., Misgav (IL)

(21) Appl. No.: 14/520,831

(22) Filed: Oct. 22, 2014

Related U.S. Application Data
(60) Provisional application No. 61/894,960, filed on Oct. 24, 2013.
(60) Provisional application No. 61/985,368, filed on Apr. 28, 2014.
(60) Provisional application No. 62/063,608, filed on Oct. 14, 2014.

(51) Int. Cl. A61K 38/18; C07K 14/505; C12N 5/071; A61K 31/573; A61K 35/36; C12N 15/86; C07K 14/56

(52) U.S. Cl. CPC A61K 38/1816; C12N 15/86; C07K 14/505; C07K 14/56; A61K 31/573; A61K 35/36; C12N 5/0625; A61K 48/00
USPC 424/85.7; 435/325; 424/93.21

(57) ABSTRACT

The present invention is directed to long-lasting therapeutic formulations and their methods of use wherein the formulation comprises a genetically modified micro-organ that comprises a nucleic acid sequence operably linked to one or more regulatory sequences. The present invention is further directed to methods providing sustained expression of therapeutic polypeptides and prolonged therapeutic effects, such as erythropoietin and interferon.

### CROSS-REFERENCE TO RELATED APPLICATIONS

This application claims priority to U.S. Provisional Application No. 61/894,960, filed Oct. 24, 2013, U.S. Provisional Application No. 61/985,368, filed Apr. 28, 2014, and U.S. Provisional Application No. 62/063,608, filed Oct. 14, 2014, each of which are incorporated by reference in their entirety.

### REFERENCE TO SEQUENCE LISTING

The instant application contains a Sequence Listing which has been submitted electronically in ASCII format and is hereby incorporated by reference in its entirety. Said ASCII copy, created on Oct. 21, 2014, is named 01118-0001-00US_SL.txt and is 321,532 bytes in size.

### FIELD OF THE INVENTION

This invention is directed to genetically modified micro-organs that provide a sustained delivery of a therapeutic polypeptide. Methods of use of a genetically modified micro-organ that provides a sustained delivery of a therapeutic polypeptide can provide a prolonged therapeutic effect to a subject in need. Therapeutic polypeptides provided by a genetically modified micro-organ of this invention include human erythropoietin and human interferon.

### BACKGROUND OF THE INVENTION

Therapeutic polypeptides can be delivered orally, transdermally, by inhalation, by injection or by depot with slow release. However, effective sustained delivery of a therapeutic polypeptide and a subsequent prolonged therapeutic effect of the therapeutic polypeptide are limited by the catabolism or inactivation that a polypeptide may be subjected in vivo, by the requirement for frequent manufacturing, purification and administration of the polypeptide, and limitations on the size of molecules that can be utilized. For some of the methods, the amount of therapeutic polypeptide varies between administrations.

Development of a purification scheme for a therapeutic polypeptide is a very lengthy process. And once purified recombinant protein has been obtained, it must be further formulated to render it stable and acceptable for introduction into animals or humans. Furthermore, even formulated, purified recombinant proteins have a finite shelf life due to maintenance and storage limitations; often requiring repeated purification and formulation of more protein. The process of developing an appropriate formulation of a therapeutic polypeptide is time consuming, difficult, and costly, as well.

For example, erythropoietin (EPO) is a therapeutic polypeptide critical for the growth and differentiation of committed erythroid progenitor cells, and a continuous low level of EPO must be maintained to meet the ongoing need to replace erythrocytes lost to senescence or blood loss. There is a progressive decline in serum EPO levels as kidney function is reduced culminating in the near universal presence of anemia in patients with end stage renal disease (ESRD). Left untreated, the anemia of chronic kidney disease (CKD) results in deterioration of cardiac function, decreased cognition, and often profound symptoms including fatigue, weakness, and lethargy. Many patients are unable to perform even normal activities of daily living. Current treatment options include the provision of Packed Red Blood Cells (PRBC) transfusions or exogenously administered recombinant human erythropoietin (rHuEPO). Most patients (greater than 90%) on hemodialysis receive intravenous (IV) or subcutaneous (SC) rHuEPO three times per week.

Other studies have suggested that the method by which rHuEPO is delivered can have a substantial effect on clinical efficacy (the increase in Hb per unit dose of rHuEPO). Despite reduced bioavailability as compared to IV administration, subcutaneous (SC) delivery of rHuEPO results in maintenance of Hb within the desired range with a 25-50% reduction in dose requirements. This is felt to be the result of serum EPO pharmacokinetics (PK) such that SC dosing allows for a longer period of time during which serum EPO levels are maintained above a critical threshold to support survival of committed erythroid precursors (burst-forming and colony forming units). Very low or rapidly declining serum EPO levels, as occurs with intermittent IV rHuEPO administration, and even with SC administered rHuEPO, is felt to contribute to the early loss of these committed erythroid precursors and the subsequent need for higher overall doses of rHuEPO.

Frequent administration of rHuEPO and its associated sharp peaks and valleys in rHuEPO levels are thought to lead to adverse health events. A method for delivering EPO that produces sustained, low levels of EPO necessary to support ongoing erythroid precursor survival would be beneficial to more effectively treat the anemia of CKD and ESRD patients at a lower overall exposure to rHuEPO.

Accordingly, there remains a need in the art for therapeutic polypeptide product formulations that provide sustained delivery of the therapeutic polypeptide for periods of time lasting for several weeks, months or more, and for methods of providing these formulations for sustained delivery in a subject in need in order to treat disease.

### SUMMARY OF THE INVENTION

This invention provides, in one embodiment, a genetically modified micro-organ that provides a sustained delivery of a therapeutic polypeptide, wherein the micro-organ comprises a vector comprising a nucleic acid sequence encoding said therapeutic polypeptide operably linked to an upstream MAR regulatory sequence, and wherein the nucleic acid further comprises at least one additional regulatory sequence, and wherein the genetically modified micro-organ provides the therapeutic polypeptide for a sustained period of at least three months, such as at least six months.

In another embodiment, the invention provides a genetically modified micro-organ that provides a sustained delivery of a therapeutic polypeptide, wherein the micro-organ comprises a helper-dependent adenoviral vector or an AAV vector comprising a nucleic acid sequence encoding said therapeutic polypeptide operably linked to an upstream MAR regulatory sequence, and wherein the nucleic acid further comprises at least one additional regulatory sequences, and wherein the genetically modified micro-organ provides the therapeutic polypeptide for a sustained period of at least three months, such as at least six months.

The therapeutic polypeptide may be human erythropoietin or human interferon. In some embodiments, the at least one or more additional regulatory sequences comprises a CAG promoter sequence, an EF1α promoter sequence, an additional MAR regulatory sequence, or a WPRE sequence.

In some embodiments, the at least one additional regulatory sequence comprises another MAR regulatory sequence and an EF1α promoter. In some embodiments, the therapeutic polypeptide is human erythropoietin and the nucleic acid encoding the erythropoietin is optimized as shown in SEQ ID NO: 2. In some embodiments, the genetically modified micro-organ is a genetically modified dermal micro-organ.

In some embodiments, a genetically modified dermal micro-organ comprising the expression cassette shown in FIG. 3 is encompassed. The expression cassette shown in FIG. 3 (see SEQ ID NO:11) specifically includes a CpG free human β-globin MAR regulatory sequence (SEQ ID NO: 6); an EF1α promoter (SEQ ID NO: 18); a gene encoding a therapeutic polypeptide to be expressed from the micro-organ, such as a gene encoding human EPO or IFN either optimized or wildtype; SV40 poly A sequence (SEQ ID NO: 9); and human IFNβ S/MAR regulatory sequence (SEQ ID NO: 5). Thus, some embodiments of the compositions and any of the methods described herein use a genetically modified micro-organ comprising a vector comprising a nucleic acid sequence comprising MAR and EF1α regulatory elements followed by the gene to be expressed followed by an SV40 poly A sequence and another MAR sequence, as illustrated in FIG. 3.

In one embodiment, a genetically modified dermal micro-organ comprising a helper-dependent adenoviral vector or an AAV vector comprising the expression cassette shown in FIG. 3 is encompassed.

In other embodiments, a genetically modified dermal micro-organ comprising the expression cassette shown in FIG. 3 is encompassed, wherein the expression cassette is CpG-free. In another embodiment, only the MAR elements and EPO gene are CpG-free.

In some embodiments, a genetically modified dermal micro-organ comprising a helper-dependent adenoviral vector or an AAV vector comprising the expression cassette shown in FIG. 3 is encompassed, wherein the expression cassette is CpG-free. In another embodiment, only the MAR elements and EPO gene are CpG-free.

In one embodiment, a genetically modified dermal micro-organ comprising nucleic acids having at least 80%, 85%, 90%, or 95% identity to the nucleic acids of SEQ ID NO: 11 is encompassed. In one embodiment, a genetically modified dermal micro-organ comprising the nucleic acids of SEQ ID NO: 11 is encompassed.

In one embodiment, a genetically modified dermal micro-organ comprising a helper-dependent adenoviral vector or an AAV vector comprising nucleic acids having at least 80%, 85%, 90%, or 95% identity to the nucleic acids of SEQ ID NO: 11 is encompassed.

In one embodiment, a genetically modified dermal micro-organ comprising a helper-dependent adenoviral vector or an AAV vector comprising the nucleic acids of SEQ ID NO: 11 is encompassed.

In one embodiment, a genetically modified dermal micro-organ comprising a helper-dependent adenoviral vector comprising the nucleic acids of SEQ ID NO: 22, or a nucleic acid that is at least 80%, 85%, 90%, or 95% identical to SEQ ID NO: 22 is encompassed.

In one embodiment, a genetically modified dermal micro-organ comprising an AAV vector comprising the nucleic acids of SEQ ID NO: 26 or 27, or a nucleic acid that is at least 80%, 85%, 90%, or 95% identical to SEQ ID NO: 26 or 27 is encompassed.

In one embodiment, a genetically modified dermal micro-organ comprising a helper-dependent adenoviral vector or an AAV vector comprising the nucleic acids of SEQ ID NO: 11 is encompassed, wherein the nucleic acids of SEQ ID NO: 11 are CpG-free. In another embodiment, only the MAR elements and EPO gene components of SEQ ID NO: 11 are CpG-free. In one embodiment, nucleic acids are at least 80%, 85%, 90%, or 95% identical to the nucleic acids of SEQ ID NO: 11 and are partially or fully CpG-free.

This invention provides, in one method embodiment, a method of treating anemia in a human subject in need over a sustained time period comprising the steps of: providing at least one genetically modified micro-organ that provides a sustained delivery of a human erythropoietin, the micro-organ comprising a vector comprising a nucleic acid sequence encoding human erythropoietin operably linked to an upstream MAR regulatory sequence, and wherein the nucleic acid optionally further comprises at least one additional regulatory sequence; determining erythropoietin secretion levels of the at least one genetically modified micro-organ in vitro; implanting the at least one genetically modified micro-organ in the human subject at an effective dosage; and measuring erythropoietin levels in the blood serum of the subject; wherein implantation of the at least one genetically modified micro-organ increases the in vivo serum erythropoietin levels in the subject over basal levels for at least three months, such as for at least six months.

This invention provides, in another method embodiment, a method of treating anemia in a human subject in need over a sustained time period comprising the steps of: providing at least one genetically modified micro-organ that provides a sustained delivery of a human erythropoietin, the micro-organ comprising a helper-dependent adenoviral vector or AAV vector comprising a nucleic acid sequence encoding human erythropoietin operably linked to an upstream MAR regulatory sequence, and wherein the nucleic acid optionally further comprises at least one additional regulatory sequence; determining erythropoietin secretion levels of the at least one genetically modified micro-organ in vitro; implanting the at least one genetically modified micro-organ in the human subject at an effective dosage; and measuring erythropoietin levels in the blood serum of the subject; wherein implantation of the at least one genetically modified micro-organ increases the in vivo serum erythropoietin levels in the subject over basal levels for at least three months.

In each of the method and non-method embodiments above, the at least one genetically modified micro-organ may be a genetically modified dermal micro-organ. In some embodiments, the optional further at least one additional regulatory sequence may comprise a CAG promoter sequence, an EF1α promoter sequence, an additional MAR regulatory sequence, or a WPRE sequence. The additional regulatory sequences may include an EF1 promoter sequence and an additional MAR regulatory sequence. In some embodiments the method of treating anemia comprises providing a genetically modified dermal micro-organ comprising a helper-dependent adenoviral vector or AAV vector comprising the nucleic acids of SEQ ID NO: 11, or a nucleic acid sequence that is at least 85%, at least 90%, or at least 95% identical to SEQ ID NO: 11.

In certain embodiments, an effective dosage of erythropoietin is about 18-150 U (or IU) erythropoietin/Kg body weight of the subject/day. In other embodiments, an effective dosage of erythropoietin is about 18-25 U (or IU) erythropoietin/Kg body weight of the subject/day, 35-45 U (or IU) erythropoietin/Kg body weight of the subject/day, or 55-65 U (or IU) erythropoietin/Kg body weight of the subject/day. In one embodiment, an implanted at least one genetically modified micro-organ provides sustained delivery of erythropoietin to a subject in need for at least three months. In another embodiment, the in vivo serum erythropoietin levels in the subject are increased over basal levels for at least six months.

In some embodiments, methods of this invention further comprise a step of measuring hemoglobin levels in the blood of the subject following the implantation of at least one genetically modified micro-organ, wherein the measured hemoglobin levels in the subject are increased and then maintained at 9-11 g/dl or 9-12 g/dl in at least 50% of the measurements for at least three months or hemoglobin levels are maintained at 9-11 g/dl or 9-12 g/dl in at least 50% of the measurements for at least three months. In some cases, the measured hemoglobin levels are 9-11 g/dl in at least 50% of the measurements for at least six months. In some cases, the measured hemoglobin levels are at least 9-11 g/dl in at least 50% of the measurements for at least three months. In some cases, the measured hemoglobin levels are at least 9-11 g/dl in at least 50% of the measurements for at least six months.

In some embodiments, methods of this invention further comprise a step of implanting at a later date to the subject, at least one additional genetically modified micro-organ that provides a sustained delivery of a human erythropoietin, the micro-organ comprising a vector comprising a nucleic acid sequence encoding human erythropoietin operably linked to an upstream MAR regulatory sequence, and wherein the nucleic acid optionally further comprises at least one or more additional regulatory sequences. In one embodiment, the at least one additional genetically modified micro-organ is a genetically modified dermal micro-organ. In some embodiments, the optional further at least one additional regulatory sequence may comprise a CAG promoter sequence, an EF1α promoter sequence, an additional MAR regulatory sequence or a WPRE sequence. In some embodiments the additional regulatory sequences include an EF1α promoter sequence and an additional MAR regulatory sequence. In some embodiments the at least one additional genetically modified dermal micro-organ comprises a helper-dependent adenoviral vector or AAV vector comprising the nucleic acids of SEQ ID NO: 11, or a nucleic acid sequence that is at least 85%, at least 90%, or at least 95% identical to SEQ ID NO: 11.

In certain embodiments of the above methods, implanting a genetically modified micro-organ comprises subcutaneous or intradermal or subdermal implantation.

This invention provides in another method embodiment a method of providing increased serum erythropoietin levels in a human subject over a sustained period of time comprising the steps of: providing at least one genetically modified micro-organ that provides a sustained delivery of a human erythropoietin, the micro-organ comprising a vector comprising a nucleic acid sequence encoding erythropoietin operably linked to an upstream MAR regulatory sequence, and wherein the nucleic acid optionally further comprises at least one additional regulatory sequence; determining erythropoietin secretion levels of the at least one genetically modified micro-organ in vitro; implanting the at least one genetically modified micro-organ in the subject at an effective dosage; and measuring erythropoietin levels in the blood serum of the subject, wherein implantation of the at least one genetically modified micro-organ increases the in vivo serum erythropoietin levels over basal levels for at least three months. In one embodiment, the in vivo serum erythropoietin levels are increased over basal levels for at least six months. In one embodiment, the at least one genetically modified micro-organ is a genetically modified dermal micro-organ. In certain embodiments, an effective dosage of erythropoietin is about 18-150 U (or IU) erythropoietin/Kg body weight of the subject/day. In other embodiments, an effective dosage of erythropoietin is about 18-25 U (or IU) erythropoietin/Kg body weight of the subject/day, 35-45 U (or IU) erythropoietin/Kg body weight of the subject/day, or 55-65 U (or IU) erythropoietin/Kg body weight of the subject/day. In some embodiments, the vector is a helper-dependent adenoviral vector or AAV vector. In some embodiments the at least one additional genetically modified dermal micro-organ comprises a helper-dependent adenoviral vector or AAV vector comprising the nucleic acids of SEQ ID NO: 11, or a nucleic acid sequence that is at least 85%, at least 90%, or at least 95% identical to SEQ ID NO: 11. In certain embodiments the at least one additional genetically modified dermal micro-organ comprises a helper-dependent adenoviral vector or AAV vector comprising the nucleic acids of SEQ ID NO: 13, SEQ ID NO: 15, or SEQ ID NO: 17, or a nucleic acid sequence that is at least 85%, at least 90%, or at least 95% identical to SEQ ID NO: 13, SEQ ID NO: 15, or SEQ ID NO: 17.

In some embodiments, the optional further at least one or more additional regulatory sequences may comprise a CAG promoter sequence, an EF1α promoter sequence, an additional MAR regulatory sequence, or a WPRE sequence. In some embodiment the additional regulatory sequences include an EF1α promoter sequence and an additional MAR regulatory sequence. In some embodiments, the methods further comprise a step of measuring hemoglobin levels in the blood of the subject following the implantation of at least one genetically modified micro-organ, wherein the measured hemoglobin levels in the subject are increased and then maintained at 9-11 g/dl or 9-12 g/dl in at least 50% of the measurements for at least three months or hemoglobin levels are maintained at 9-11 g/dl or 9-12 g/dl in at least 50% of the measurements for at least three months. In some cases, the measured hemoglobin levels are 9-11 g/dl or 9-12 g/dl in at least 50% of the measurements for at least six months. In some embodiments the method of providing increased serum erythropoietin levels in a human subject over a sustained period of time comprises providing the human subject with a genetically modified dermal micro-organ comprising a helper-dependent adenoviral vector or AAV vector comprising the nucleic acids of SEQ ID NO: 11, or a nucleic acid sequence that is at least 85%, at least 90%, or at least 95% identical to SEQ ID NO: 11.

The methods of this invention in which erythropoietin is delivered to the subject may be used with a subject having: renal failure, chronic renal failure, chemotherapy induced anemia, anemia as a result of HIV treatments, microangiopathic hemolytic anemia, anemia as a result of prematurity, an inflammatory condition including rheumatoid arthritis, an infection, anemia associated with cancers including multiple myeloma and non-Hodgkin lymphoma, hematopoietic stem cell disorders, anemia associated with myelodysplastic syndrome (MDS), sickle cell anemia or thalassemia, including alpha-thalassemia, beta-thalassemia, alpha-thalassemia trait, alpha-thalassemia minor, alpha-thallasemia intermedia, alpha-thalassemia major, beta-thalassemia trait, beta-thalassemia minor, beta-thalassemia intermedia, beta-thalassemia major, Constant Spring, Cooley's Anemia, hemoglobin Bart hydrops fetalis, and hemoglobin E thalassemia; or a subject in need of accelerated erythroid repopulation after bone marrow transplantation; or any combination thereof. In some embodiments a subject suffering from chronic renal failure is suffering from chronic kidney disease (CKD) or end stage renal disease (ESRD).

In the methods of this invention in which erythropoietin is delivered to the subject, the in vivo serum erythropoietin levels are increased over basal levels for at least six months, or the in vivo serum erythropoietin levels have decreased decay rates over basal levels, or the genetically modified micro-organ has a prolonged therapeutic effect such as sustained and increased percent hematocrit over basal levels, or the genetically modified micro-organ is capable of autoregulating hemoglobin levels.

This invention provides, in another method embodiment, a method of treating hepatitis in a human subject in need over a sustained time period comprising the steps of: providing at least one genetically modified micro-organ that provides a sustained delivery of a human interferon, the micro-organ comprising a vector comprising a nucleic acid sequence encoding human interferon operably linked to an upstream MAR regulatory sequence, and wherein the nucleic acid optionally further comprises at least one additional regulatory sequence; determining interferon secretion levels of the at least one genetically modified micro-organ in vitro; implanting the at least one genetically modified micro-organ in the human subject at an effective dosage; and measuring interferon levels in the blood serum of the subject; wherein implantation of the at least one genetically modified micro-organ increases the in vivo serum interferon levels in the subject over basal levels for at least three months. The interferon may be human interferon α (alpha), β (beta), γ (gamma) or λ (lambda).

In one embodiment, in a method of this invention, the at least one genetically modified micro-organ is a genetically modified dermal micro-organ. In some embodiments, the optional further at least one additional regulatory sequences may comprise a CAG promoter sequence, an EF1α promoter sequence, an additional MAR regulatory sequence or a WPRE sequence. In some embodiment the additional regulatory sequences include an EF1α promoter sequence and an additional MAR regulatory sequence. In some embodiments the vector is a helper-dependent adenoviral vector or AAV vector. In some embodiments the at least one additional genetically modified dermal micro-organ comprises a helper-dependent adenoviral vector or AAV vector comprising the nucleic acids of SEQ ID NO: 23, or a nucleic acid sequence that is at least 85%, at least 90%, or at least 95% identical to SEQ ID NO: 23.

In one embodiment, methods of this invention provide that an implanted at least one genetically modified micro-organ provides sustained delivery of interferon to a subject in need for at least three months. In another embodiment, the in vivo serum interferon levels are increased over basal levels for at least six months.

In some embodiments, methods of this invention further comprise a step of implanting at a later date to the subject, at least one additional genetically modified micro-organ that provides a sustained delivery of a human interferon, such as human interferon α, β, γ, or λ, the micro-organ comprising a vector comprising a nucleic acid sequence encoding human interferon operably linked to an upstream MAR regulatory sequence, and wherein the nucleic acid further comprises at least one or more additional regulatory sequences. In one embodiment, the at least one additional genetically modified micro-organ is a genetically modified dermal micro-organ. In some embodiments the vector is a helper-dependent adenoviral vector or AAV vector. In some embodiments the vector comprising the nucleic acids of SEQ ID NO: 23 or SEQ ID NO: 25, or nucleic acids having at least 80%, 85%, 90%, or 95% identity to SEQ ID NO: 23 or SEQ ID NO: 25.

In certain embodiments, implanting a genetically modified micro-organ comprises subcutaneous or intradermal or subdermal implantation.

Where interferon is delivered to the subject, methods of this invention may be used with a subject selected from a group consisting of: hepatitis B, hepatitis C or hepatitis D; or any combination thereof.

This invention provides in one embodiment a method of providing increased serum interferon levels in a human subject over a sustained period of time comprising the steps of: providing at least one genetically modified micro-organ that provides a sustained delivery of a human interferon, the micro-organ comprising a vector comprising a nucleic acid sequence encoding interferon operably linked to an upstream MAR regulatory sequence, and wherein the nucleic acid optionally further comprises at least one or more additional regulatory sequences; determining interferon secretion levels of the at least one genetically modified micro-organ in vitro; implanting the at least one genetically modified micro-organ in the subject at an effective dosage; and measuring interferon levels in the blood serum of the subject, wherein implantation of the at least one genetically modified micro-organ increases the in vivo serum interferon levels over basal levels for at least three months. In one embodiment, the in vivo serum interferon levels are increased over basal levels for at least six months. In one embodiment, the at least one genetically modified micro-organ is a genetically modified dermal micro-organ. In some embodiments the vector is a helper-dependent adenoviral vector or AAV vector.

In some embodiments, the optional further at least one additional regulatory sequences may comprise a CAG promoter sequence, an EF1α promoter sequence, an additional MAR regulatory sequence or a WPRE sequence. In some embodiments the at least one additional genetically modified dermal micro-organ comprises a helper-dependent adenoviral vector or AAV vector comprising the nucleic acids of SEQ ID NO: 23, or a nucleic acid sequence that is at least 85%, at least 90%, or at least 95% identical to SEQ ID NO: 23.

In certain embodiments, each method described herein may further comprise a step of administering methylprednisolone, e.g., Depo-Medrol®, following the implanting step, wherein the administering is by subcutaneous injection around one or more of the implanted genetically modified micro-organs. In one embodiment, the site of administration is no more than 5 mm away from a genetically modified micro-organ implantation site. In one embodiment, a dose of methylprednisolone, e.g., Depo-Medrol®, administered is about 12 mg per genetically modified micro-organ implantation site.

This invention provides in one embodiment, a method of providing a therapeutic polypeptide to a subject in need over a sustained time period, the method comprising the steps of: providing at least one genetically modified micro-organ expressing and secreting a therapeutic polypeptide; determining in vitro secretion levels of the therapeutic polypeptide from said at least one genetically modified micro-organ; implanting the at least one genetically modified micro-organ in a subject; and administering methylprednisolone, e.g., Depo-Medrol®, by subcutaneous injection around one or more of the implanted genetically modified micro-organs; wherein the method delivers the therapeutic polypeptide to the subject for a sustained time period of at least three months. In one embodiment, the at least one genetically modified micro-organ is a genetically modified dermal micro-organ. In one embodiment, the methylprednisolone is administered by subcutaneous injection. In one embodiment, an injection of methylprednisolone is no more than 5 mm away from the implantation site of a genetically modified micro-organ. In one embodiment, a sustained period of time is at least six months.

In any of the above composition and method embodiments, the genetically modified micro-organs, such as genetically modified dermal micro-organs, may be obtained from explants and may maintain the three-dimensional structure of the tissue or organ from which they were derived. In any of the above composition and method embodiments, the genetically modified micro-organs, such as genetically modified dermal micro-organs, may be autologous.

Any of the above methods may further comprise a step of applying at least one topical steroid following said methylprednisolone administration step, to an area of skin at and around a genetically modified micro-organ implantation site. In one embodiment, the at least one topical steroid is betamethasone dipropionate (Diprolene®), clobetasol propionate (Temovate®, Clobex®, Olux®-E Olux®, Cormax®), halobetasol propionate (Ultravate®), flucinonide (Vanos®), flurandrenolide (Cordran®), diflorasone diacetate (Psorcon®, ApexiCon®), amcinonide (Cyclocort®, Amcort®), betamethason dipropionate (Dipronsone®, Diprolene® AF), halcinonide (Halog®), fluocinonide (Lidex®), diflorasone diacetate (ApexiCon®, Florone®), desoximetasone (Topicort®), triamcinolone acetonide (Kenalog®, Triderm®, Aristocort® HP, Aristocort® A, Aristocort®), betamethasone valerate (Valisone®, Luxiq®, Beta-Val®), fluticasone propionate (Cutivate®), fluocinonide (Lidex®-E), mometasone furoate (Elocon®), fluocinolone acetonide (Synalar®, Capex®, Derma-Smoothe®/FS), mometasone furoate (Elocon®), hydrocortisone valerate (Westcort®), clocortolone pivalate (Cloderm®), prednicarbate (Dermatop®), desonide (DesOwen®, Tridesilon®, Desonate®, LoKara®, Verdeso®), hydrocortisone butyrate (Locoid®, Lipocream®, Cortizone®-10), hydrocortisone probutate (Pandel®), alclometasone dipropionate (Aclovate®), or hydrocortisone (base) (Hytone®, Nutracort®, Texacort®, Cortaid®, Synacort®, Aquinil® HC, Sarnol® HC, Cortizone®-10, Noble, Scalp relief), or any combination thereof. In one embodiment, the at least one topical steroid is a betamethasone valerate. In one embodiment, the at least one topical steroid is a topical glucocorticoid.

In one embodiment, methods of this invention comprise application of at least one topical steroid daily starting a week following implantation of an at least one genetically modified micro-organ.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 provides an illustration of the CAG-wt-hEPO expression cassette.

FIG. 2 provides an illustration of the CAG-opt-hEPO expression cassette.

FIG. 3 provides an illustration of the MAR-EF1α-opt-hEPO expression cassette.

FIG. 4 provides an illustration of the MAR CAG-opt-hEPO-WPRE expression cassette.

FIG. 5 illustrates in vitro erythropoietin secretion profiles from EPO Genetically Modified Micro-organs (GMMOs) transduced with different helper-dependent adenoviral vector (HDAd) constructs.

FIG. 6 illustrates skin to skin variability of in vivo erythropoietin secretion profiles from EPO GMMOs transduced with different HDAd vectors. HA-131, HA-132 and HA-138 represent independent analysis of hEPO secretion from different tummy tuck skin sources.

FIGS. 7a and 7b illustrate sustained serum delivery in vivo with decreased rate of decay (% decrease from peak). FIG. 7a illustrates sustained serum hEPO concentrations (IU/ml) and resultant increased and sustained percent (%) hematocrit in SCID mice following implantation of EPO GMMOs transduced with different HDAd constructs. FIG. 7b illustrates that rate of decay of delivered EPO is decreased in SCID mice producing EPO from expression cassettes including at least one MAR regulatory element. Control mice showed constant % hematocrit levels of around 50% (data not shown).

FIGS. 8a and 8b illustrate administration of methylprednisolone decreases rate of decay in vivo of a therapeutic polypeptide. FIG. 8a illustrates that weekly injections of methylprednisolone, e.g., Depo-Medrol®, around implanted GMMOs resulted in sustained secretion of hEPO compared with secretion in the absence of methylprednisolone administration. FIG. 8b illustrates reduced decay rate of serum hEPO concentrations (IU/ml) compared with decay rate in the absence of methylprednisolone administration. Experiments were carried out in SCID mice implanted with two EPO GMMOs per mouse, decay rate was measured from the initial peak level observed six days following implantation of the GMMOs. GMMOs implanted expressed hEPO from HDAd-CAG-wtEPO vector.

FIGS. 9a and 9b illustrate sustained serum hEPO delivery in vivo with decreased rate of decay (% decrease from peak). FIG. 9a illustrates hEPO serum levels and hematocrit percent in SCID mice following implantation of EPO GMMOs and methylprednisolone, e.g., Depo-Medrol® (Depo), administration. FIG. 9b illustrates that rate of decay of delivered EPO is decreased in SCID mice producing EPO from expression cassettes including at least one MAR regulatory element, wherein methylprednisolone is administered The results also show increased serum hEPO levels when using expression cassettes including at least one MAR regulatory element.

FIGS. 10a and 10b illustrate sustained serum hEPO delivery in vivo with decreased rate of decay (% decrease from peak). FIG. 10a illustrates hEPO serum levels and hematocrit percentage in SCID mice following implantation of EPO GMMOs and methylprednisolone, e.g., Depo-Medrol®, administration every second week. FIG. 10b illustrates that rate of decay of delivered EPO is decreased in SCID mice producing EPO from expression cassettes including at least one MAR regulatory element, wherein methylprednisolone is administered every second week for eight weeks.

FIG. 11 illustrates the relationship between erythropoietin (EPO) dose of EPO GMMO administered and net peak increase in serum EPO levels above baseline. EPO GMMO expressed hEPO from CAG-wtEPO cassette.

FIG. 12 is a schematic flowchart illustrating the steps for evaluating the safety and effectiveness of administering EPO GMMOs for treating anemia in hemodialysis patients, according to certain embodiments of the invention.

FIGS. 13a and 13b illustrate embodiments of methylprednisolone (Depo-Medrol®) injection sites relative to EPO GMMOs implantation sites. In FIG. 13a, line markings between the cross-hashes define EPO GMMO implantation sites. “Stars” indicate Depo-Medrol injection sites. In FIG. 13b, the longer non-dashed lines indicate location of GMMO between the 2 tattoo dots, and the shorter dashed lines indicate the sites for Depo-Medrol® subcutaneous infiltration aside and along the GMMO.

FIG. 14 illustrates one embodiment of a study design for EPO GMMO treatment assessment of efficacy and safety.

FIGS. 15a and 15b illustrate representative data from a patient enrolled in the clinical trial described in Example 8. FIG. 15a shows serum rHuEPO levels in mIU/ml during the run-in period (pre-implantation). FIG. 15b shows serum Hb in g/dL during this same time period (run-in; pre-implantation). Without the EPO GMMOs of the present invention, supratherapeutic doses of rHuEPO are required to maintain hemoglobin levels in the target range (9-11 g/dL and less than about 12 g/dL).

FIGS. 16a, 16b, and 16c illustrate representative data from another patient enrolled in the clinical trial described in Example 8, wherein the patient was provided three EPO GMMO's of the invention. FIG. 16a shows serum eEPO levels in mIU/ml over time (approximately 110 days from implantation). FIG. 16b shows serum Hb levels in g/dL over this same time period (approximately 110 days from implantation). FIG. 16c shows reticulocyte (10⁶/L) over time (approximately 110 days from implantation). FIGS. 16a, 16b, and 16c show that the EPO GMMOs of the invention, when administered to humans, effectively raise serum EPO and Hb levels to therapeutically relevant levels for at least 110 days, with minimal peaks and troughs associated with subcutaneous rHuEPO injections. This representative patient stabilized after 30 days and target Hb was reached with approximately 100 times lower C_{MAX} than rHuEPO (compare to FIGS. 15a and 15b). The data also indicates that patients receiving the EPO GMMOs of the invention may be auto-regulating their serum Hb levels.

FIGS. 17a and 17b illustrate representative data from another patient enrolled in the clinical trial described in Example 8, wherein the patient was provided two EPO GMMOs of the invention. FIG. 17a shows serum eEPO levels in mIU/ml over time (approximately 70 days from implantation). FIG. 17b shows serum Hb levels in g/dL over this same time period (approximately 70 days from implantation). FIGS. 17a and 17b show that the EPO GMMOs of the invention, when administered to humans, effectively raise serum EPO and Hb levels to therapeutically relevant levels for at least 70 days, with minimal peaks and troughs associated with subcutaneous rHuEPO injections. This representative patient stabilized after 45 days and their target Hb was reached with approximately 100 times lower C_{MAX} than rHuEPO. The data also indicates that patients receiving the EPO GMMOs of the invention may be auto-regulating their serum Hb levels.

FIGS. 18a and 18b illustrate representative data from another patient enrolled in the clinical trial described in Example 8, wherein the patient was provided one EPO GMMOs of the invention. FIG. 18a shows serum eEPO levels in mIU/ml over time (approximately 30 days from implantation). FIG. 18b shows serum Hb levels in g/dL over this same time period (approximately 30 days from implantation). FIGS. 18a and 18b show that the EPO GMMOs of the invention, when administered to humans, effectively raise serum EPO and Hb levels to therapeutically relevant levels for at least 30 days, with minimal peaks and troughs associated with subcutaneous rHuEPO injections.

FIG. 19 illustrates the results of a study to compare HDAd-CAG-opt-hIFNalpha and HDAd-MAR-EF1a-opt-hIFNalpha when expressed in the GMMOs of the invention. GMMOs were prepared with HDAd-CAG-opt-hIFNalpha and HDAd-MAR-EF1a-opt-hIFNalpha and two GMMOs were implanted to the back of each SCID mouse. Serum hIFNalpha in ng/ml was assessed over about 185 days. The results indicate that the HDAd-MAR-EF1a-opt-hIFNalpha construct provides increased secretion levels of interferon and longer in vivo duration of secretion as compared to HDAd-CAG-opt-hIFNalpha.

FIG. 20 illustrates the results of a study to compare HDAd-CAG-opt-hIFNalpha and HDAd-MAR-EF1a-opt-hIFNalpha when expressed in the GMMOs of the invention when Depo-Medrol® was also administered to the implantation site. GMMOs were prepared with HDAd-CAG-opt-hIFNalpha and HDAd-MAR-EF1a-opt-hIFNalpha and two GMMOs were implanted to the back of each SCID mouse. At the day of implantation and every two weeks post implantation for the duration of the experiment, 1 mg (100 μl) of Depo-Medrol® was administered to the implantation site of each GMMO. Serum hIFNalpha in ng/ml was assessed over about 185 days. The results indicate that the HDAd-MAR-EF1a-opt-hIFNalpha construct provides increased efficacy in vivo as compared to HDAd-CAG-opt-hIFNalpha, and that Depo-Medrol® administration may further stabilize secretion levels and improve duration of secretion of the HDAd-MAR-EF1a-opt-hIFNalpha construct.

FIG. 21 represents hEPO isoelectric focusing results obtained from 3 human skin samples transduced with the HDAd-CAG-wt-hEPO or HDAd-MAR-EF1α-opt-hEPO vectors. A similar isoelectric pattern was observed for each skin sample, indicating similar hEPO isoforms are produced from the GMMOs, even though transduced with the two different vectors. As expected, a sample taken from un-transduced MO did not show any hEPO signal, validating the specificity of the method. When hEPO GMMOs were compared to EPO controls, the tested samples had more basic EPO isoforms than the recombinant EPO standards and the human urinary erythropoietin standard. “NIBSC” is a human urinary EPO control; BRP is a Biological Reference Preparation (BRP batches 1 and 2a) control from the European Pharmacopoeia Commission (an equimolecular mixture of rHuEPO: epoetin a and b); Aranesp is Recombinant human EPO control; “optEPO” is a GMMO comprising HDAd-MAR-EF1α-opt-hEPO; “wtEPO” is a GMMO comprising HDAd-CAG-wt-hEPO.

FIGS. 22a and 22b show the effect of the length of in-vitro GMMO processing time on its in-vivo performance post implantation into SCID mice. FIG. 22a shows in-vivo hEPO serum levels post implantation of GMMOs transduced with HDAd-MAR-EF1α-opt-hEPO that were processed in-vitro for 3 days (left bars) or for 9 days (right bars). FIG. 22b shows in-vivo hEPO serum levels post implantation of GMMOs transduced with AAV-LK19-MAR-CAG-opt-hEPO-WPRE that were processed in-vitro for 3 days (left bars) or for 9 days (right bars). Bars indicate the hEPO concentration measured by ELISA in mouse serum. Values are mean±SD, n=5 for each group.

FIG. 23 shows in-vivo hEPO serum levels post implantation of GMMOs transduced with AAV-LK19-MAR-CAG-opt-hEPO-WPRE that were processed in-vitro for 3 days (left bars) or for 6 days (right bars). Bars indicate the hEPO concentration measured by ELISA in mouse serum.

FIG. 24 shows in-vivo hEPO serum levels post implantation of GMMOs transduced with AAV-LK19-MAR-CAG-opt-hEPO-WPRE that were processed in-vitro for 3 days (left bars) or for 10 days (right bars).

FIG. 25 shows in-vivo hEPO serum levels post implantation of GMMOs transduced with HDAd-MAR-EF1α-opt-hEPO that were processed in-vitro for 1 day (left bars), 3 days (second from left bars), 9 days (third from left bars), or for 13 days (fourth from left bars).

FIG. 26 shows in vivo IFNa serum levels post implantation of GMMOs transduced with HDAd-MAR-EF1a-opt IFNa that were processed in vitro for 2 days (left bars), 4 days (middle bars), or 9 days (right bars).

FIG. 27 shows in vitro secretion profiles of GMMOs transduced with AAV comprising S/MAR-CAG-opt-hEPO-WPRE and MAR-EF1alpha-opt-hEPO expression cassettes. The in vitro secretion profile of GMMOs transduced with AAV comprising MAR-EF1alpha-opt-hEPO expression cassette was significantly improved as compared to S/MAR-CAG-opt-hEPO-WPRE expression cassettes. Four representative experiments are shown (h-255; h-256; h-259; and h-268).

FIG. 28 shows in vivo secretion profiles of GMMOs transduced with AAV comprising S/MAR-CAG-opt-hEPO-WPRE and MAR-EF1alpha-opt-hEPO expression cassettes. The in vivo secretion profile of GMMOs transduced with AAV comprising MAR-EF1alpha-opt-hEPO expression cassettes were significantly improved as compared to S/MAR-CAG-opt-hEPO-WPRE expression cassettes (approximately 5-fold increase).

FIGS. 29a and 29b shows in vitro performance of two different types of AAV vectors comprising ssAAV8-MAR-CAG-optEPO-WPRE and scAAV8-MAR-CAG-optEPO-WPRE. FIG. 29a shows hEPO secretion in vitro from EPO GMMOs comprising ssAAV8-MAR-CAG-optEPO-WPRE. FIG. 29b shows hEPO secretion in vitro from EPO GMMOs comprising scAAV8-MAR-CAG-optEPO-WPRE.

FIG. 30 shows in vitro performance of a different AAV vector, AAV1/2, comprising AAV1/2-MAR-CAG-wtEPO. hEPO was secreted in vitro from EPO GMMOs comprising AAV1/2-MAR-CAG-wtEPO.

FIG. 31 shows in vitro performance of a different AAV vector, AAV1, comprising scAAV2/1-CAG-wtEPO. hEPO was secreted in vitro from EPO GMMOs comprising scAAV2/1-CAG-wtEPO.

FIGS. 32a and 32b show in vitro performance of two different AAV vectors, ssAAV2i8 and scAAV2i8, comprising ssAAV2i8-MAR-CAG-optEPO-WPRE and sc AAV2i8-CAG-optEPO. FIG. 32a shows hEPO was secreted in vitro from EPO GMMOs comprising ssAAV2i8-MAR-CAG-optEPO-WPRE. FIG. 32b shows hEPO was secreted in vitro from EPO GMMOs comprising sc AAV2i8-CAG-optEPO.

FIG. 33 shows in vitro performance of a different AAV vector, AAV-LK19, comprising MAR-CAG-optEPO-WPRE expression cassettes. hEPO was secreted in vitro from EPO GMMOs comprising AAV-LK19-MAR-optEPO-WPRE.

FIG. 34 shows in vitro skin to skin performance variability of EPO GMMO comprising AAV-LK19-MAR-CAG-optEPO-WPRE. Different donor MOs are indicated by “HA-number.” AAV-LK19-MAR-CAG-optEPO-WPRE comprising GMMOs secreted EPO in each skin type tested.

FIG. 35 shows the long term in vitro secretion profile of an EPO GMMO comprising AAV-LK19-MAR-CAG-optEPO-WPRE. Relatively steady hEPO was observed for more than 6 months.

FIG. 36 shows the effect of in vitro processing time on the GMMOs in vivo performance. AAV-LK19 comprising MAR-CAG-optEPO-WPRE expression cassettes. AAV-LK19 comprising MAR-CAG-optEPO-WPRE were used to transduce MOs and the transduced MOs were maintained in vitro for 3, 10, or 14 days prior to implantation. As seen with HDAd transduced MOs, AAV transduced MOs also secreted higher levels of hEPO and provided an increased % hematocrit when the in vitro processing time was reduced from 14 to 10 to 3 days.

FIG. 37 shows the long term in vivo secretion profile of EPO GMMOs comprising AAV-LK19 expressing MAR-CAG-optEPO-WPRE cassettes. EPO GMMOs comprising AAV-LK19-MAR-CAG-optEPO-WPRE were implanted into SCID mice and serum hEPO and % hematocrit were assessed. The results show at least 241 days of steady hEPO secretion.

FIG. 38 shows the in vivo performance of EPO GMMOs comprising HDAd-MAR-EF1a-opt-hEPO compared to AAV-LK19-MAR-CAG-opt-hEPO-WPRE. While the HDAd transduced GMMOs initially had higher in vivo secretion levels than the AAV transduced GMMOs, by about 3 months the measured levels of EPO in the serum was about the same. When observed for longer periods of time, the AAV transduced GMMOs maintained EPO levels in the serum while the level of EPO in the serum of mice transduced with HDAd GMMOs declined (data not shown).

### DESCRIPTION OF THE SEQUENCES

DESCRIPTION OF THE SEQUENCES

| SEQ | |
|---|---|
| ID NO: | SEQUENCE DESCRIPTION |
| 1 | Homo sapiens erythropoietin (nucleic acid) |
| 2 | Optimized human EPO (nucleic acid) |
| 3 | Homo sapiens erythropoietin (amino acid) |
| 4 | human IFNbeta S/MAR (nucleic acid) |
| 5 | 5′ region of human IFNbeta S/MAR MAR (nucleic acid) |
| 6 | CpG free human beta-globin MAR (nucleic acid) |
| 7 | CAG promoter (nucleic acid) |
| 8 | WPRE (nucleic acid) |
| 9 | Simian virus 40 (nucleic acid) |
| 10 | pdelta28-MAR-EF1alpha-optEPO (nucleic acid) |
| 11 | MAR-EF1alpha-opt-hEPO Expression Cassette |
| 12 | pdelta28-MAR-CAG-opt-hEPO-WPRE (nucleic acid) |
| 13 | S/MAR-CAG-opt-hEPO-WPRE cassette (nucleic acid) |
| 14 | pdelta-CAG-wt-hEPO (nucleic acid) |
| 15 | CAG-opt-hEPO cassette (nucleic acid) |
| 16 | pdelta-CAG-opt-hEPO (nucleic acid) |
| 17 | CAG-wt-hEPO cassette (nucleic acid) |
| 18 | EF1alpha promoter (nucleic acid) |
| 19 | Homo sapiens interferon, alpha 2 (IFNA2) (nucleic acid) |
| 20 | Optimized human IFN (nucleic acid) |
| 21 | Homo sapiens interferon, alpha 2 (IFNA2) (amino acid) |
| 22 | pdelta28-MAR-EF1alpha-optIFNalpha (nucleic acid) |
| 23 | MAR-EF1alpha-optIFNalpha expression cassette (nucleic |
| | acid) |
| 24 | pdelta28-MAR-CAG-optIFNalpha-WPRE (nucleic acid) |
| 25 | MAR-CAG-optIFNalpha-WPRE expression cassette |
| | (nucleic acid) |
| 26 | pAAV-LK19-MAR-CAG-opt-hEPO-WPRE (nucleic acid) |
| 27 | pAAV-LK19-MAR-EF1alpha-opt-hEPO (nucleic acid) |
| 28 | pAd-CAG-Opt INFa |

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, the instant invention is directed to genetically modified, tissue-based micro-organs that provide a sustained delivery of a therapeutic polypeptide in vitro and in vivo, wherein the micro-organ comprises a vector comprising a nucleic acid sequence encoding a therapeutic polypeptide, operably linked to one or more regulatory sequences, and methods of use of the micro-organ. In certain embodiments, a genetically modified micro-organ of this invention expresses and secretes the therapeutic polypeptide for a sustained time period, such as for at least three or at least six months. In one embodiment, the regulatory sequence includes at least one MAR sequence. In one embodiment, implantation of the genetically modified micro-organ is accompanied by administration of an anti-inflammatory agent, for instance, methylprednisolone. In one embodiment, a therapeutic polypeptide is erythropoietin (“EPO”). In another embodiment, a therapeutic polypeptide is an interferon (“IFN”), such as IFN alpha, IFN beta, IFN gamma, or IFN lambda. In some embodiments, methods of use of long-lasting therapeutic formulations of this invention provide a prolonged therapeutic effect to a subject in need. In some embodiments, methods of use of long-lasting therapeutic formulations of this invention provide a sustained therapeutic effect to a subject in need. In certain embodiments the micro-organ comprises a helper-dependent viral vector or an AAV vector comprising a nucleic acid sequence encoding a therapeutic polypeptide.

The invention provides, in certain embodiments, an at least one genetically modified micro-organ (“GMMO”) that expresses and secretes a therapeutic polypeptide, the micro-organ (“MO”; as used herein “MO” also refers to the plural: micro-organs) comprising a vector, such as a helper-dependent adenovirus vector (“HDAd”) or adeno-associated virus (AAV) vector, the vector comprising a nucleic acid sequence encoding the therapeutic polypeptide operably linked to an upstream MAR regulatory sequence, and wherein the nucleic acid further comprises one or more additional regulatory sequences. In certain embodiments, methods of use of at least one GMMO of this invention provide a prolonged therapeutic effect to a subject in need. In certain embodiments, methods of use of an at least one GMMO provide a sustained delivery of a therapeutic polypeptide to a subject in need. As used herein, the terms “a GMMO” and “an at least one GMMO” may refer to “a therapeutic formulation”, wherein the therapeutic formulation may include multiple GMMOs expressing the same therapeutic polypeptide.

In one embodiment, a GMMO comprises a vector, such as an HDAd or AAV vector, comprising a nucleic acid sequence encoding a therapeutic polypeptide operably linked to an upstream MAR regulatory sequence, wherein the nucleic acid optionally further comprises at least one additional regulatory sequence, and wherein the at least one genetically modified micro-organ expresses the therapeutic polypeptide for a sustained period of at least three months, at least four months, at least five months, or at least six months.

In one embodiment, a GMMO comprises a helper-dependent adenoviral vector or an AAV vector comprising a nucleic acid sequence encoding a therapeutic polypeptide operably linked to an upstream MAR regulatory sequence, wherein the nucleic acid optionally further comprises at least one additional regulatory sequence, and wherein the at least one genetically modified micro-organ expresses the therapeutic polypeptide for a sustained period of at least three months, at least four months, at least five months, or at least six months.

In one embodiment, a therapeutic polypeptide is human erythropoietin or human interferon, such as interferon-α, interferon-β, interferon-γ, or interferon-λ.

In some embodiments, any of the genetically modified micro-organs (GMMOs) of this invention, in any form or embodiment as described herein, may be used in methods of this invention to deliver a therapeutic polypeptide over a sustained time period. As used herein in certain embodiments, the term “therapeutic-GMMO” refers to genetically modified micro-organs such as genetically modified dermal micro-organs that secrete a therapeutic polypeptide. As used herein in certain embodiments, the terms “EPO-GMMO” and “hEPO-GMMO” refer to genetically modified micro-organs such as genetically modified dermal micro-organs that secrete a human EPO, and may be used interchangeably having all the same meanings and qualities. As used herein in certain embodiments, the terms “IFN-GMMO” and “hIFN-GMMO” refer to genetically modified micro-organs such as genetically modified dermal micro-organs that secrete a human IFN, and may be used interchangeably having all the same meanings and qualities.

In one embodiment, an MO is derived from autologous tissue. For example, an EPO GMMO may comprise autologous tissue harvested from the same subject in which it may be implanted following transduction of the tissue with an EPO vector.

In some embodiments, the invention provides a therapeutic formulation and methods of use thereof, where the formulation comprises at least one GMMO.

The term “MO” as used herein, refers in one embodiment, to an isolated tissue or organ structure derived from or identical to an explant that has been prepared in a manner conducive to cell viability and function. In one embodiment, the explant is an intact tissue explant. In one embodiment, an MO maintains at least some in vivo structures of the tissue or organ from which it was isolated. In another embodiment, an MO maintains cell-to-cell interactions, similar to those of the tissue or organ from which it is obtained. In one embodiment, an MO is an intact, isolated tissue sample. In another embodiment, the MO retains the micro-architecture and the three dimensional structure of the tissue or organ from which it was derived and has dimensions selected so as to allow passive diffusion of adequate nutrients and gases to cells within the micro-organ and diffusion of cellular waste out of the cells of the micro-organ so as to minimize cellular toxicity and concomitant cell death due to insufficient nutrition and/or accumulation of waste. In one embodiment, an MO is a sliver of dermal tissue, i.e., a dermal micro-organ (“DMO”).

In one embodiment, the GMMO is a genetically modified dermal micro-organ. Dermal micro-organs (“DMO”) may comprise a plurality of dermis components, where in one embodiment dermis is the portion of the skin located below the epidermis. These components may comprise fibroblast cells, epithelial cells, other cell types, bases of hair follicles, nerve endings, sweat and sebaceous glands, and blood and lymph vessels. In one embodiment, a dermal micro-organ may comprise some fat tissue, wherein in another embodiment, a dermal micro-organ may not comprise fat tissue.

In some embodiments of the invention, the dermal micro-organ may contain tissue of a basal epidermal layer and, optionally, other epidermal layers of the skin. In other embodiments, the dermal micro-organ does not include basal layer tissue. In another embodiment of the invention, the dermal micro-organ does not include epidermal layers. In yet another embodiment, the dermal micro-organ contains an incomplete epidermal layer. In still another embodiment, the dermal micro-organ may contain a few layers of epidermal tissue. In still another embodiment, the dermal micro-organ may contain invaginations of the epidermis into the dermis. In another embodiment, a dermal micro-organ does not include a complete epidermal layer. In a further embodiment, the dermal micro-organ may include additional components such as sweat glands and/or hair follicles.

In one embodiment of the invention, the DMO includes the entire cross-section of the dermis. In another embodiment of the invention, the dermal micro-organ includes part of the cross-section of the dermis. In a further embodiment, the DMO includes most of the cross section of the dermis, namely, most of the layers and components of the dermis including the papillary and reticular dermis. In a further embodiment, the DMO includes primarily dermal tissue, but may also include fat tissue. In some embodiments of the invention, the DMO does not produce keratin or produces a negligible amount of keratin, thereby preventing the formation of keratin cysts following implantation in a recipient, for example, following subcutaneous or intradermal implantation. Further details regarding dermal micro-organs, including methods of harvesting, maintaining in culture, and implanting said dermal micro-organs, are described in PCT Patent Applications WO2004/099363 and WO2013/118109.

In one embodiment, an MO is 1-2 mm in diameter and 30-40 mm in length. In another embodiment, the diameter of an MO may be, for example, 1-3 mm, 1-4 mm, 2-4 mm, 0.5-3.5 mm, 1.5-2.5 or 1.5-10 mm. In another embodiment the diameter of an MO may be, for example, approximately 2 mm or approximately 1.5 mm. In another embodiment, the diameter is less than 10 mm, and in another embodiment, the length is less than 1.5 cm. In another embodiment, the length of the MO may be 5-100 mm, 10-60 mm, 20-60 mm, 20-50 mm, 20-40 mm, 20-100 mm, 30-100 mm, 40-100 mm, 50-100 mm, 60-100 mm, 70-100 mm, 80-100 mm, or 90-100 mm. In another embodiment, the length of the MO may be approximately 20 mm, approximately 30 mm, approximately 40 mm, or approximately 50 mm. In one embodiment, the length may be greater than 100 mm. In one embodiment, a DMO of this invention has a diameter of about 2 mm and a length of about 30 mm. In another embodiment, a DMO of this invention has a diameter of about 2 mm and a length of about 40 mm.

In one embodiment, an MO is an explant. In one embodiment, an MO is tissue-derived. In another embodiment, an MO is a section or portion or part of a tissue. In another embodiment, an MO is a section or portion or part of an organ. In one embodiment, an MO is an intact section or portion or part of an organ or a tissue. An MO can be distinguished from a skin graft, in one embodiment, in that it is specifically designed to survive for long periods of time in vivo and in vitro and, in another embodiment, in that its dimensions are specifically selected so as to allow passive diffusion of adequate nutrients and gases to cells within the MO and diffusion of cellular waste out of the cells of the MO, which in one embodiment minimizes cellular toxicity and concomitant cell death due to insufficient nutrition and/or accumulation of waste. Thus, in one embodiment, an MO is not a skin graft. In another embodiment, an MO can be distinguished from a collection of isolated cells, which in one embodiment, are grown on a natural or artificial scaffold, in that MO maintain the micro-architecture and the three dimensional structure of the tissue or organ from which they were derived. Thus, in one embodiment, an MO is not one or more cell types grown on a scaffold or within a gel or on or within a sponge.

A detailed description of some embodiments of MO can be found in US2003/0152562.

The therapeutic GMMO of the present invention unexpectedly showed an increased expression profile and a reduced decay rate of a therapeutic polypeptide, for example EPO in vivo. In one embodiment, the EPO GMMO of this invention provided an extended sustained expression and secretion of EPO in vitro and in vivo, compared with previously known EPO GMMOs. An advantage of an extended sustained expression of a therapeutic polypeptide is that it may lead to a prolonged or sustained therapeutic effect. The term therapeutic polypeptide includes functional fragments of therapeutic polypeptides.

The term “a” or “an” means more than one. For example, “a genetically modified micro-organ”of the present invention can be one or more genetically modified micro-organs.

As used herein, the term “explant” refers, in one embodiment, to a tissue or organ or a portion thereof removed from its natural growth site in an organism and placed in a culture medium for a period of time. In one embodiment, the tissue or organ is viable, in another embodiment, metabolically active, or a combination thereof. In one embodiment, the explant is intact. As used herein, the term “explant” may, in some embodiments, be used interchangeably with “micro-organ” or “micro-organ explant”.

As used herein, the term “microarchitecture” refers, in one embodiment, to a characteristic of the explant in which some or all of the cells of the tissue explant maintain, in vitro, physical and/or functional contact with at least one cell or non-cellular substance with which they were in physical and/or functional contact in vivo.

In some embodiments, MO explants maintain the three-dimensional structure of the tissue or organ from which they were derived. In one embodiment, MO explants retain the spatial interactions, e.g. cell-cell, cell-matrix and cell-stromal interactions, and the orientation of the tissue from which they were derived. In one embodiment, preservation of spatial interactions such as described above permit the maintenance of biological functions of the explant, such as secretion of autocrine and paracrine factors and other extracellular stimuli, which in one embodiment, provide long term viability to the explant. In one embodiment, at least some of the cells of the MO explant maintain, in vitro or in vivo after implantation, their physical and/or functional contact with at least one cell or non-cellular substance with which they were in physical and/or functional contact in vivo. In one embodiment, “some of the cells” refers to at least about 50%, in another embodiment, at least about 60%, in another embodiment at least about 70%, in another embodiment, at least about 80%, and in another embodiment, at least about 90% or more of the cells of the population. In another embodiment, the cells of the explant maintain at least one biological activity of the organ or tissue from which they are isolated.

Examples of mammals from which the MO can be isolated include humans and other primates, swine, including wholly or partially inbred swine (e.g., miniature swine, and transgenic swine), rodents, etc. MO may be processed from tissue from a variety of organs, which in one embodiment is the skin, the dermis, the lymph system, the pancreas, the liver, the gallbladder, the kidney, the digestive tract, the respiratory tract, the reproductive system, the urinary tract, the lung, the bladder, the cornea, the prostate, the bone marrow, the thymus, the spleen, or a combination thereof. Explants from these organs may comprise islet of Langerhans, hair follicles, glands, epithelial or connective tissues, or a combination thereof, arranged in a microarchitecture similar to the microarchitecture of the organ from which the explant was obtained. In one embodiment, the microarchitecture of the organ from which the explant was obtained may be discerned or identified in the MO explant using materials, apparati, and/or methods known in the art.

The term “about” is intended herein to encompass numbers or ranges of numbers that differ from the recited number or range but that would fall within the recited number or range upon rounding to reduce the number of significant digits, or encompass deviations caused by measurement errors.

In some embodiments, the term “consisting essentially of” refers to a GMMO, whose only elements are those indicated, however, other compounds may be included, for example, for stabilizing, preserving, etc. the formulation, or as excipients or pharmaceutically inactive ingredients, that are not involved directly in the therapeutic effect of the GMMO.

Further, when referring to methods or method steps, “consisting essentially of” includes the recited elements but excludes other elements or steps that may have an essential significant effect on the performance of the method or step.

In one embodiment, the phrase “gene product” refers to proteins or polypeptides. In one embodiment, the gene product encoded by the nucleic acid molecule is the desired gene product to be supplied to a subject. Examples of such gene products include proteins, peptides, glycoproteins and lipoproteins normally produced by cells of the recipient subject.

In some embodiments, the vector of and for use in the methods of the present invention comprises a nucleic acid sequence operably linked to one or more regulatory sequences, wherein said nucleic acid sequence encodes a therapeutic polypeptide. In another embodiment, the vector consists essentially of such a nucleic acid sequence, and in another embodiment, the vector consists of such a nucleic acid sequence. In one embodiment, the nucleic acid operably linked to one or more regulatory sequences comprises the nucleic acids of SEQ ID NO: 1, or a nucleic acid that is at least 80%, 85%, 90%, or 95% identical to SEQ ID NO: 1. In another embodiment, the nucleic acid operably linked to one or more regulatory sequences comprises the nucleic acids of SEQ ID NO: 2, or a nucleic acid that is at least 80%, 85%, 90%, or 95% identical to SEQ ID NO: 2. In yet another embodiment, the nucleic acid operably linked to one or more regulatory sequences comprises the nucleic acids of SEQ ID NO: 19, or a nucleic acid that is at least 80%, 85%, 90%, or 95% identical to SEQ ID NO: 19. In still another embodiment, the nucleic acid operably linked to one or more regulatory sequences comprises the nucleic acids of SEQ ID NO: 20, or a nucleic acid that is at least 80%, 85%, 90%, or 95% identical to SEQ ID NO: 20.

Any methodology known in the art can be used for genetically altering the micro-organ explant. Any one of a number of different vectors can be used in embodiments of this invention, such as viral vectors, plasmid vectors, linear DNA, etc., as known in the art, to introduce an exogenous nucleic acid fragment encoding a therapeutic agent into target cells and/or tissue. Examples of virus vectors include adenovirus vectors, helper-dependent adenovirus vectors, adeno-associated virus vectors, and retroviral vectors. These vectors can be inserted, for example, using infection, transduction, transfection, calcium-phosphate mediated transfection, DEAE-dextran mediated transfection, electroporation, liposome-mediated transfection, biolistic gene delivery, liposomal gene delivery using fusogenic and anionic liposomes (which are an alternative to the use of cationic liposomes), direct injection, receptor-mediated uptake, magnetoporation, ultrasound, or any combination thereof, as well as other techniques known in the art (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). The polynucleotide segments encoding sequences of interest can be ligated into an expression vector system suitable for transducing mammalian cells and for directing the expression of recombinant products within the transduced cells. The introduction of the exogenous nucleic acid fragment is accomplished by introducing the vector into the vicinity of the micro-organ. Once the exogenous nucleic acid fragment has been incorporated into the cells using any of the techniques described above or known in the art, the production and/or the secretion rate of the therapeutic agent encoded by the nucleic acid fragment can be quantified. In one embodiment, the term “exogenous” refers to a substance that originated outside, for example a nucleic acid that originated outside of a cell or tissue.

The term “vector” or “expression vector” as used herein refers to a carrier molecule into which a nucleic acid sequence can be expressed.

The GMMOs herein comprise a helper-dependent adenoviral vector (“HDAD”, “HD” or “HDAd” or “HD-Ad”) or an adeno-associated virus (AAV) vector. A helper-dependent adenoviral vector may be a gutless, gutted, mini, fully deleted, high-capacity, A, or pseudo adenovirus. It may also be deleted of all viral coding sequences except for sequences supporting DNA replication, which in one embodiment, comprise the adenovirus inverted terminal repeats and packaging sequence (ψ). In another embodiment, HDAd express no viral proteins. In one embodiment, a HDAd comprises only the cis-acting elements of the adenovirus required to replicate and package the vector DNA. In one embodiment, a HDAd comprises approximately 500 bp of wild-type adenovirus sequence. In another embodiment, the adenoviral vector additionally comprises stuffer DNA. In one embodiment, the stuffer DNA is mammalian DNA. In one embodiment, the HDAd vector is a non-replicating vector.

In one embodiment, HDAd display high-efficiency in vivo and in vitro transduction, high-level transgene expression, are able to maintain long-term transgene expression, in one embodiment, by avoiding chronic toxicity due to residual expression of viral proteins, or a combination thereof. In another embodiment, HDAd have high titer production, efficient infection of a broad range of cell types, the ability to infect dividing and nondividing cells, or a combination thereof. In yet another embodiment, a HDAd for use in the methods of the instant invention does not induce a strong adaptive immune response to an implanted GMMO, which in one embodiment, is characterized by the generation of adeno-specific MHC class I restricted CD8 cytotoxic T lymphocytes (CTL) in immunocompetent hosts, which in one embodiment, would limit the duration of transgene expression and in another embodiment, would result in adenovirus vector clearance within several weeks. In still another embodiment, a HDAd for use in the methods of the instant invention does not induce high cytotoxic T cell levels (as may be measured in one embodiment by positive CD8 staining, as is known in the art), and, in another embodiment, does not induce high helper T cell levels (as may be measured in one embodiment by positive CD4 stain, as is known in the art).

In another embodiment, HDAd have a lower risk of germ line transmission and insertional mutagenesis that may cause oncogenic transformation, because the vector genome does not integrate into the host cell chromosomes. In one embodiment, the cloning capacity of HDAd is very large (in one embodiment, approximately 37 kb, in another embodiment, approximately 36 kb), allowing for the delivery of whole genomic loci, multiple transgenes, and large cis-acting elements to enhance, prolong, and regulate transgene expression.

In one embodiment, the HDAd system for use with the compositions and in the methods of the present invention is similar to that described in Palmer and Ng, 2003 (Mol Ther 8:846) and in Palmer and Ng, 2004 (Mol Ther 10:792).

An adeno-associated virus (AAV) vector may be single stranded or double stranded and may be of any serotype.

A vector comprising a nucleic acid encoding a therapeutic polypeptide of the instant invention may be introduced into a micro-organ, for example by transduction. There are a number of techniques known in the art for introducing cassettes and/or vectors into cells, for affecting the methods of the present invention, such as, but not limited to: direct DNA uptake techniques, and virus, plasmid, linear DNA or liposome mediated transduction, receptor-mediated uptake and magnetoporation methods employing calcium-phosphate mediated and DEAE-dextran mediated methods of introduction, electroporation or liposome-mediated transfection, (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals).

The present invention, in certain embodiments, involves genetically modified micro-organs comprising a helper-dependent adenoviral vector or AAV vector comprising a nucleic acid sequence encoding a therapeutic polypeptide operably linked to an upstream MAR regulatory sequence, and wherein said nucleic acid optionally further comprises at least one additional regulatory sequence.

“Regulatory sequences” or “regulatory elements” herein mean nucleotide sequences which regulate expression of a gene product (e.g., promoter, stabilizing sequences and enhancer sequences). In some embodiments, the additional regulatory sequences may comprise a MAR sequence (or a second MAR sequence), a CAG promoter sequence, an EF1α promoter sequence or a WPRE sequence. More generally, regulatory sequences may be selected based upon the type of cell in which the gene product is to be expressed and the desired level of expression of the gene product. For example, a promoter known to confer cell-type specific expression of a gene linked to the promoter can be used. Alternatively, a regulatory element that can direct constitutive expression of a gene in a variety of different cell types, such as a viral regulatory element, can be used. Examples of viral promoters commonly used to drive gene expression include those derived from polyoma virus, Adenovirus 2, cytomegalovirus (CMV) and Simian Virus 40, and retroviral LTRs. Alternatively, a regulatory element which provides inducible expression of a gene linked thereto can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S. and White, J. H. (1993) Proc. Nati Acad. Sci. USA 90:5603-5607), synthetic ligand-regulated elements (see, e.g., Spencer, D. M. et al (1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89:1014-10153). Additional tissue-specific or inducible regulatory systems which may be developed can also be used in accordance with this invention.

The term “promoter” refers to a DNA sequence, which, in one embodiment, is operably linked upstream of the coding sequence and is important for basal and/or regulated transcription of a gene.

As used here, the term “operably linked” refers to a regulatory sequence placed in a functional relationship with another nucleotide sequence, e.g., a gene encoding a therapeutic polypeptide. For example, if the regulatory sequence is a promoter sequence and if a coding sequence is operably linked to the promoter sequence, this generally means that the promoter may promote transcription of the coding sequence. Operably linked means that the DNA sequences being linked are often contiguous and, where necessary to join two protein coding regions, contiguous and in reading frame. However, some regulatory sequences may be operably linked to but not contiguous with the coding sequences whose expression they promote. For example, enhancers may function when separated from the promoter by several kilobases and intronic sequences may be of variable length.

As defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

In one embodiment, a promoter of the compositions and for use in the methods of the present invention is a regulatable promoter. In another embodiment, a regulatable promoter refers to a promoter whereby expression of a gene downstream occurs as a function of the occurrence or provision of specific conditions that stimulate expression from the particular promoter. In some embodiments, such conditions result in directly turning on expression, or in other embodiments, remove impediments to expression. In some embodiments, such conditions result in turning off, or reducing expression.

In one embodiment, such conditions may comprise specific temperatures, nutrients, absence of nutrients, presence of metals, or other stimuli or environmental factors as will be known to one skilled in the art. In one embodiment, a regulatable promoter may be regulated by galactose (e.g. UDP-galactose epimerase (GAL10), galactokinase (GAL1)) or glucose (e.g. alcohol dehydrogenase II (ADH2)), or phosphate (e.g. acid phosphatase (PHO5)). In another embodiment, a regulatable promoter may be activated by heat shock (heat shock promoter) or chemicals such as IPTG or Tetracycline, or others, as will be known to one skilled in the art. It is to be understood that any regulatable promoter and conditions for such regulation is encompassed by the vectors, nucleic acids and methods of this invention, and represents an embodiment thereof.

GMMOs according to the invention comprise an HdAd or AAV vector comprising a nucleic acid sequence encoding a therapeutic polypeptide operably linked to an upstream scaffold/matrix attachment (S/MAR) sequence, also known as a MAR sequence. The terms “S/MAR” and “MAR” are used interchangeably throughout this application, having all the same meanings and qualities. S/MAR sequences are transcription enhancing sequences that have been shown to have a stabilizing effect in vivo on transgene expression (Klehr et al. (1991). Biochemistry 30: 1264-1270). S/MAR-based plasmids can function as stable episomes in primary human fibroblast-like cells, supporting long-term transgene expression. However, S/MAR regulatory elements do not display universal behavior in all cell types. In one embodiment, a vector of this invention comprises at least one S/MAR sequence. In another embodiment, a vector comprises at least two S/MAR sequences. S/MAR sequences within a vector may be the same or different. In one embodiment, an S/MAR sequence comprises SEQ ID NO: 4. In another embodiment, an S/MAR sequence comprises SEQ ID NO: 5. In yet another embodiment, an S/MAR sequence comprises SEQ ID NO: 6. In still another embodiment, an S/MAR sequence comprises any S/MAR sequence known in the art.

A variety of additional regulatory sequences may also be included in the HdAd vector, such as a CAG promoter sequence, an EF1α promoter sequence or a WPRE sequence. In one embodiment, a further regulatory sequence of this invention may comprise a constitutive promoter. Known constitutive promoters include SV40, CMV, UBC, EF1alpha, PGK and CAG. Promoters are known to vary considerably from one another in their strength dependent on cell type transduced and growth conditions Studies indicate that promoter activities might be restricted to specific cell lineages, suggesting the need to carefully select and test promoters for constitutive gene expression.

In one embodiment, an additional regulatory sequence of the instant invention may comprise a CMV promoter, while in another embodiment; the regulatory sequence may comprise a CAG promoter. In one embodiment, a CAG promoter is a composite promoter that combines the human cytomegalovirus immediate-early enhancer and a modified chicken beta-actin promoter and first intron. In one embodiment, a CAG promoter comprises SEQ ID NO: 7. In one embodiment, a CAG promoter comprises any CAG promoter known in the art.

In one embodiment, an additional regulatory sequence of this invention comprises an EF1α promoter. The EF1α gene has a housekeeping function in all cells and is expressed to high levels. Due to its indispensable housekeeping function in all cells, EF1α promoter expression is relatively insulated from changes in cell physiology and is cell type independent. In one embodiment, an EF1α promoter comprises SEQ ID NO: 18. In another embodiment, an EF1α promoter comprises any EF1α promoter known in the art.

In one embodiment, an additional regulatory sequence may comprise a simian virus (SV)-40 polyadenylation sequence, which in one embodiment, is the mechanism by which most messenger RNA molecules are terminated at their 3′ ends in eukaryotes. In one embodiment, the polyadenosine (poly-A) tall protects the mRNA molecule from exonucleases and is important for transcription termination, for export of the mRNA from the nucleus, and for translation. In another embodiment, a formulation of the present invention may comprise one or more regulatory sequences. In one embodiment, a poly-A tail sequence comprises SEQ ID NO: 9.

In one embodiment, an additional regulatory sequence of this invention comprises a woodchuck hepatitis virus post-transcriptional regulation element (WPRE). WPRE have been shown to enhance expression in the context of adenoviral vectors as well other viral vectors (Zanta-Boussif et al. (2009) Gene Therapy 16, 605-619; Kingsman et al., (2005) Gene Therapy 12, 3-4. WPRE sequences were shown to stimulate expression when subcloned in the sense orientation between the transgene and the poly(A) sequence. In one embodiment, a WPRE regulatory sequence is located between a sequence encoding a therapeutic polypeptide of this invention, for instance a sequence encoding EPO, and a poly(A) sequence. In another embodiment, a WPRE regulatory sequence is located between a sequence encoding IFN and a poly(A) sequence. In one embodiment, a WPRE sequence comprises SEQ ID NO: 8. In another embodiment, a WPRE sequence comprises any WPRE sequence known in the art.

In one embodiment, a nucleic acid sequence encoding a therapeutic polypeptide operably linked to an upstream MAR regulatory sequence comprises SEQ ID NO: 11. In another embodiment, a nucleic acid sequence encoding a therapeutic polypeptide operably linked to an upstream MAR regulatory sequence comprises SEQ ID NO: 13. In yet another embodiment, a nucleic acid sequence encoding a therapeutic polypeptide operably linked a regulatory sequence comprises SEQ ID NO: 15. In still another embodiment, a nucleic acid sequence encoding a therapeutic polypeptide operably linked to an upstream MAR regulatory sequence comprises SEQ ID NO: 17. In a further embodiment, a nucleic acid sequence encoding a therapeutic polypeptide operably linked an upstream MAR regulatory sequence comprises SEQ ID NO: 23. In another embodiment, a nucleic acid sequence encoding a therapeutic polypeptide operably linked an upstream MAR regulatory sequence comprises SEQ ID NO: 25.

In one embodiment, an at least one genetically modified micro-organ of this invention comprises a helper-dependent adenoviral vector comprising a nucleic acid sequence encoding an erythropoietin operably linked to an upstream MAR regulatory sequence, and wherein said nucleic acid optionally further comprises at least one or more additional regulatory sequences, and wherein the at least one genetically modified micro-organ expresses said therapeutic polypeptide for a sustained period of at least three months.

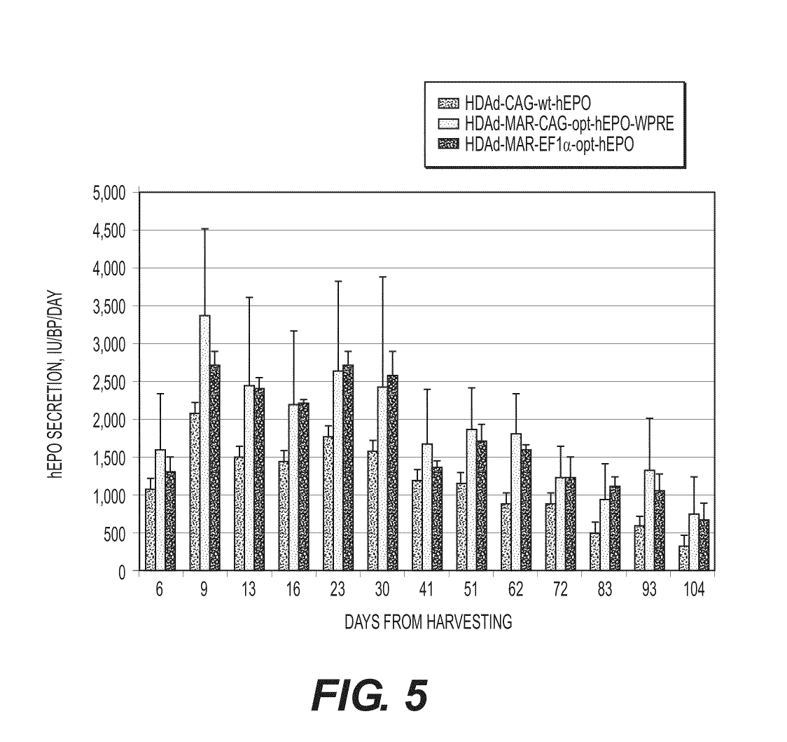

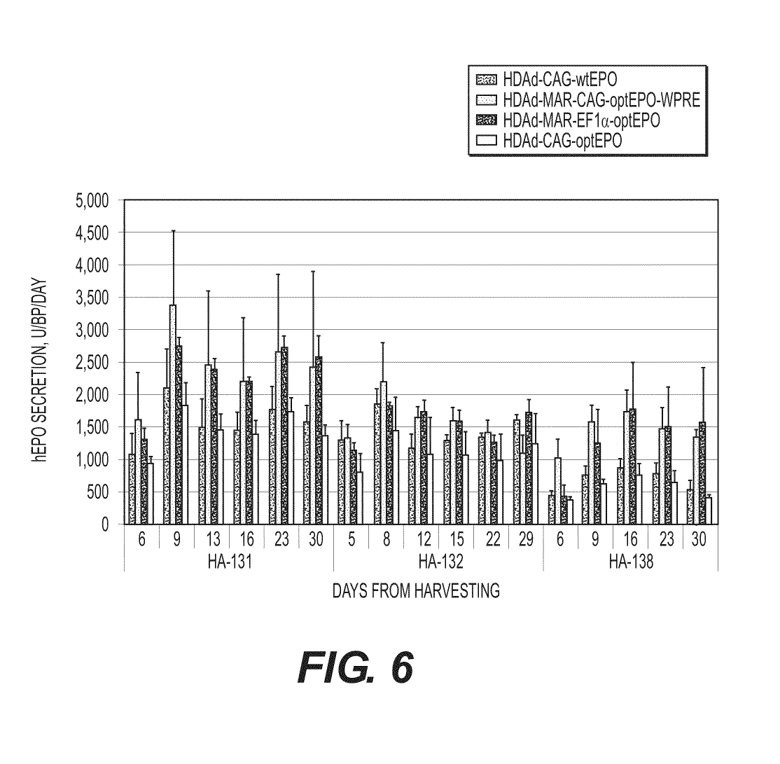

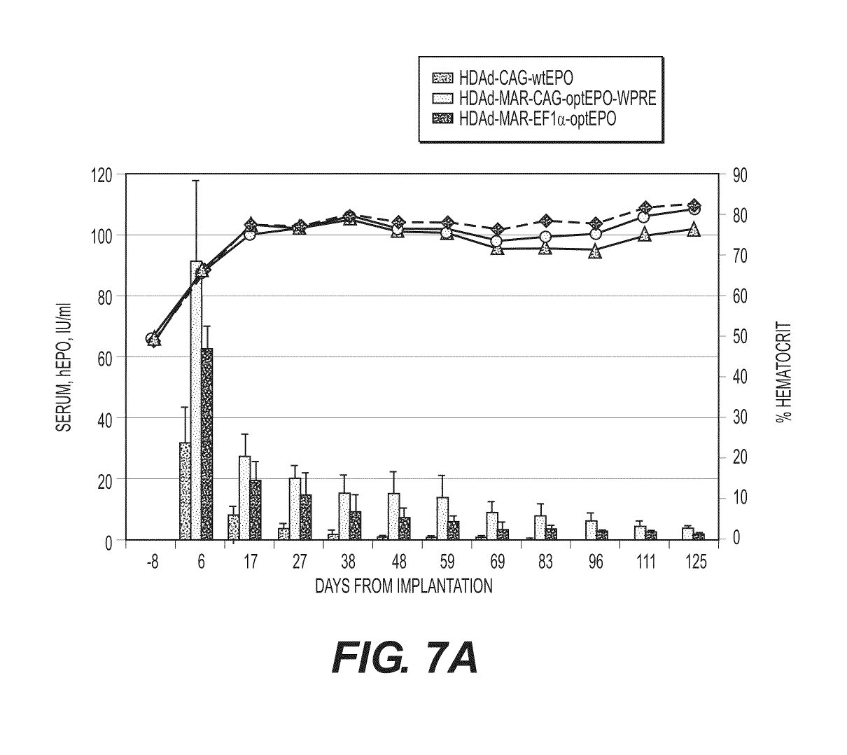

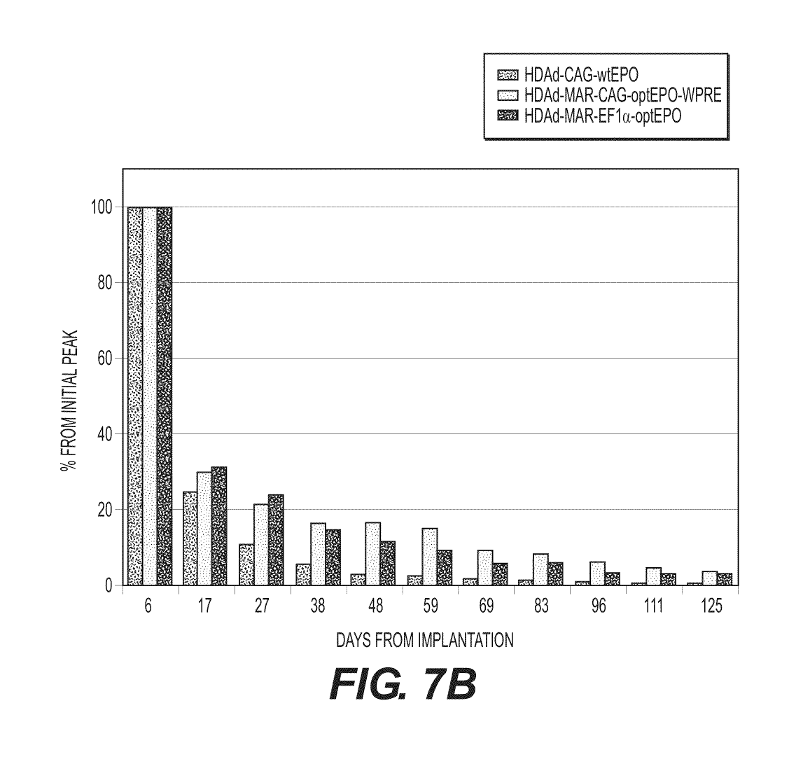

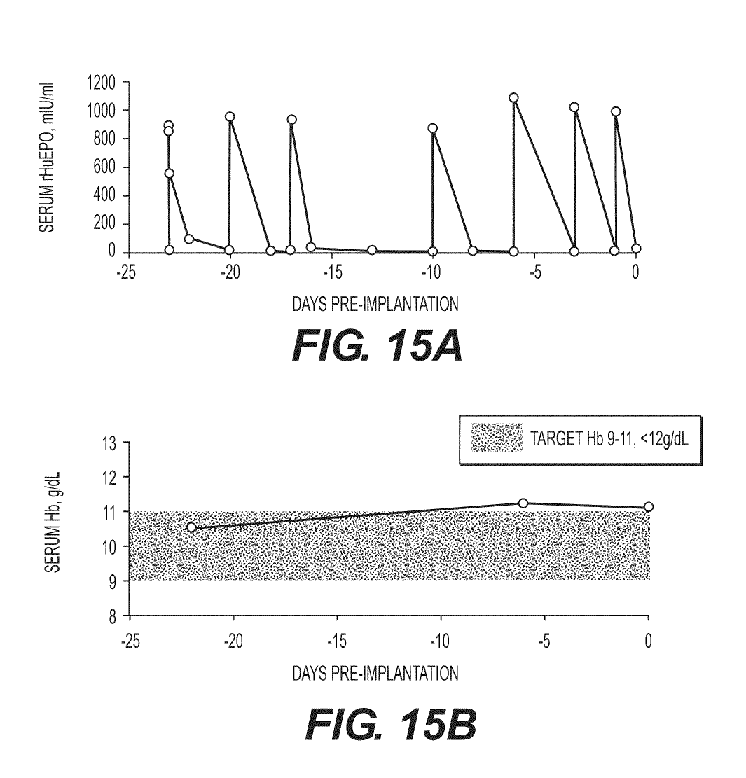

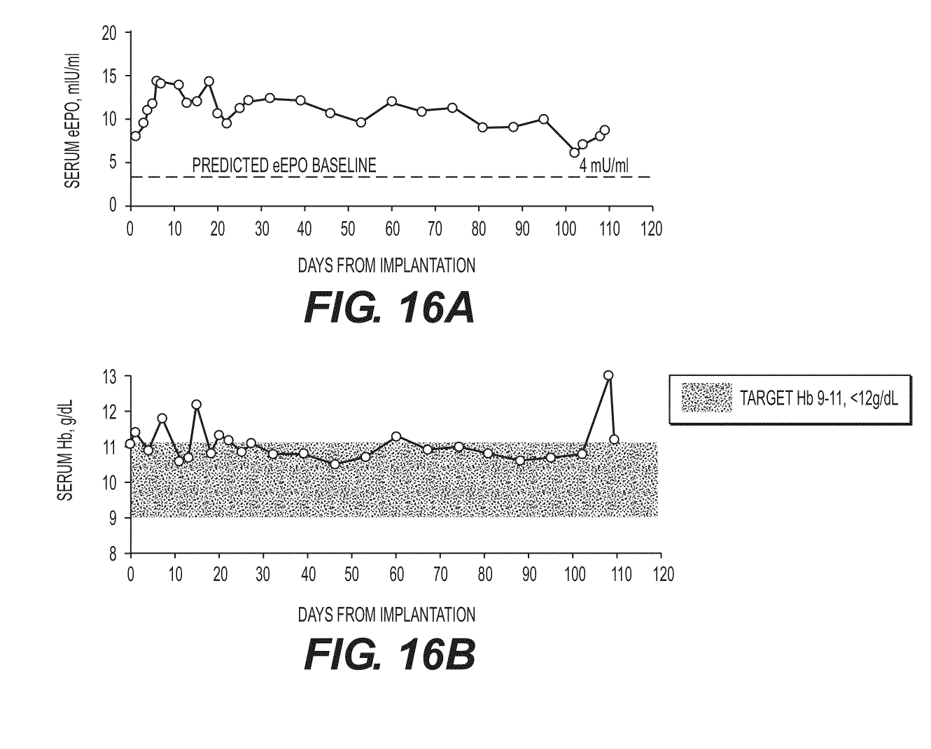

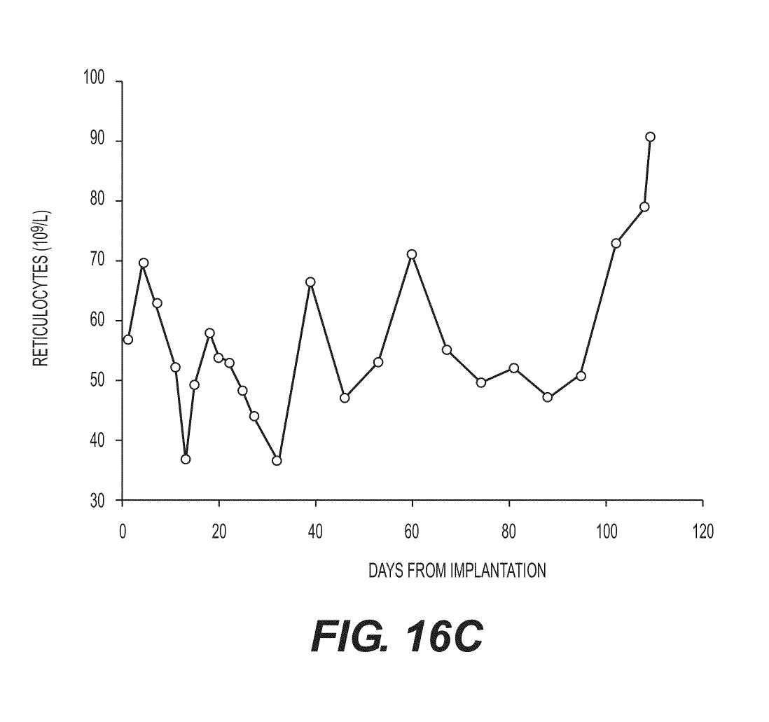

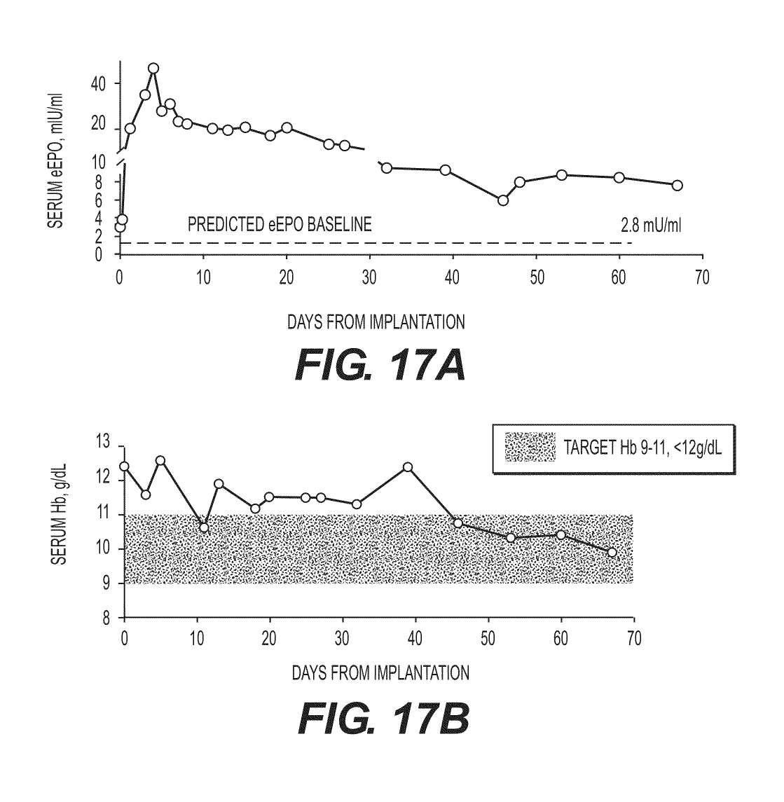

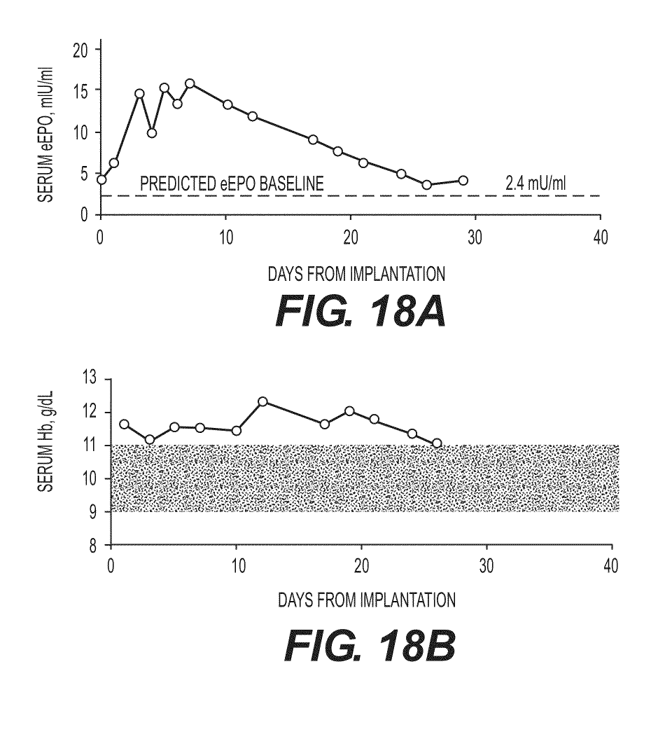

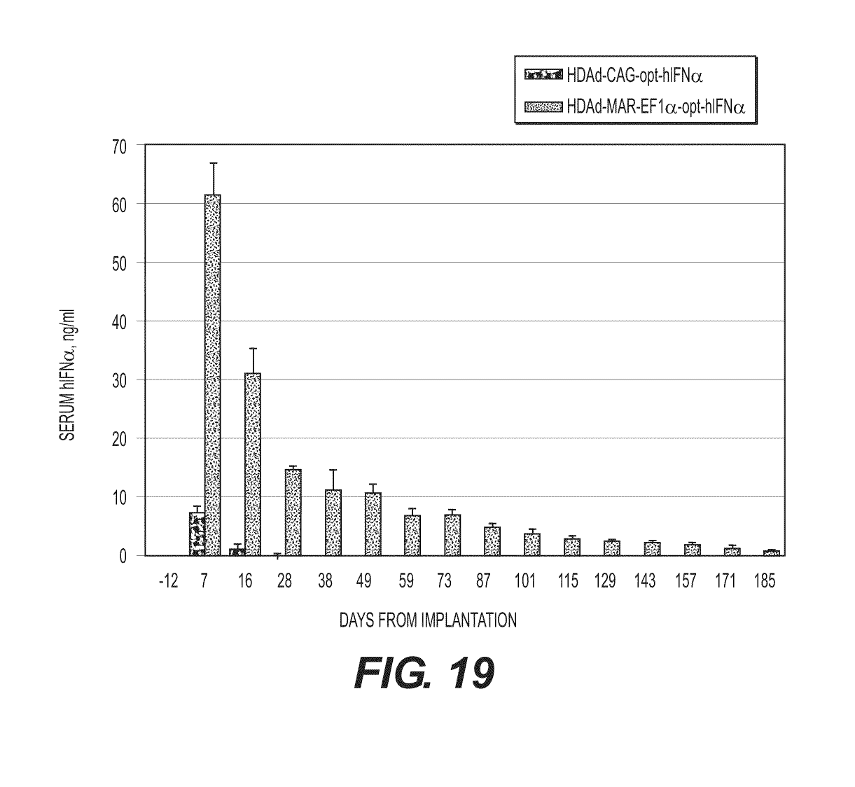

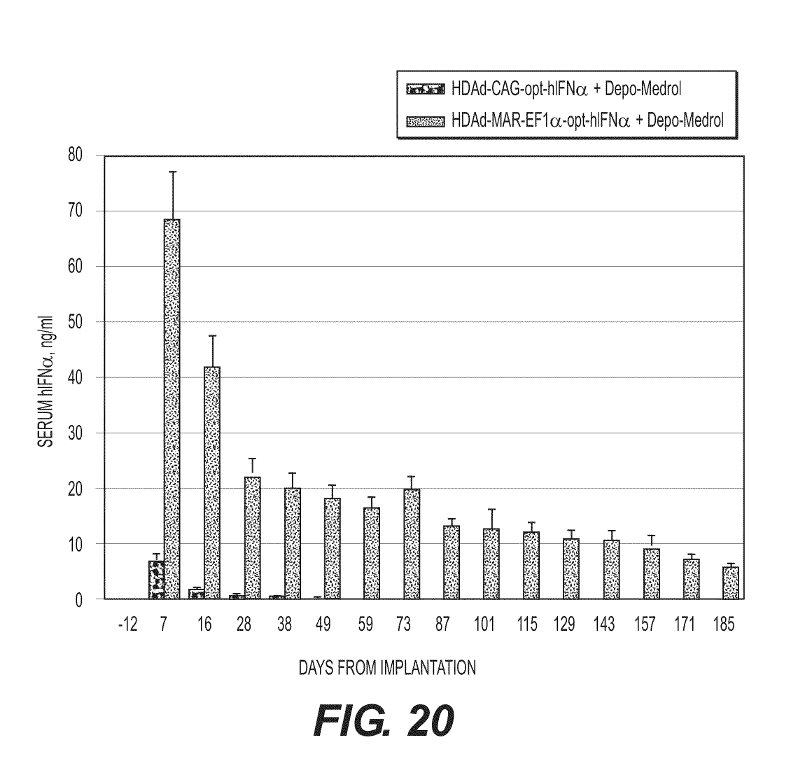

In one embodiment, an at least one GMMO of this invention demonstrates sustained in vitro expression levels of a therapeutic polypeptide, e.g., EPO (FIGS. 5 and 6). In certain embodiments, an at least one GMMO of the instant invention demonstrates sustained in vivo expression levels of a therapeutic polypeptide, e.g., EPO, decreased decay rates of the therapeutic polypeptide, e.g., EPO, and a prolonged therapeutic effect, e.g., sustained increased and maintains percent hematocrit (FIGS. 7a, 7b, 8a, 8b, 9a, 9b, 10a and 10b). In other embodiments, an at least one GMMO of the instant invention autoregulates hemoglobin levels (see, FIGS. 15-18, for example, where following a drop in hemoglobin you see a rise in serum EPO levels, increased reticulocytes, and then a rise in hemoglobin).

In one embodiment, regulatory elements comprised in a vector of this invention include at least one S/MAR sequence, a CAG promoter, a WPRE sequence and a poly(A) sequence. In another embodiment, regulatory elements comprised in a vector of this invention include at least a CAG promoter and a poly(A) sequence. In yet another embodiment, regulatory element comprised in a vector of this invention include at least an S/MAR sequence, a EF1α promoter, a WPRE sequence and a poly(A) sequence. In still another embodiment, regulatory element comprised in a vector of this invention include at least two S/MAR sequences, a EF1α promoter and a poly(A) sequence. In a further embodiment, regulatory elements comprised in a vector of this invention include at least two different S/MAR sequences and an EF1α promoter, wherein one of the S/MAR sequences is a B globin s/MAR sequence.

Certain embodiments of this invention provide a method of treating anemia in a human subject in need over a sustained time period comprising the steps of: providing an at least one GMMO of this invention that provides a sustained delivery human erythropoietin, the micro-organ comprising a vector, such as a helper-dependent adenoviral vector or adeno-associated virus vector, comprising a nucleic acid sequence encoding human erythropoietin operably linked to an upstream MAR regulatory sequence, and wherein the nucleic acid optionally further comprises at least one additional regulatory sequence; determining erythropoietin secretion levels of the at least one genetically modified micro-organ in vitro; implanting said at least one GMMO in the human subject at an effective dosage; and measuring erythropoietin levels in the serum of the subject; wherein implantation of the at least one GMMO increases the in vivo serum erythropoietin levels over basal levels for at least three months, such as for at least six months.

In one embodiment, methods of this invention use at least one GMMO comprising at least one or more additional regulatory sequences selected from the group consisting of a MAR sequence, a CAG promoter sequence, an EF1α promoter sequence and a WPRE sequence. In one embodiment, methods of this invention use at least one GMMO comprising a nucleic acid sequence comprises SEQ ID NO: 11 or SEQ ID NO: 13. In one embodiment, methods of this invention use at least one GMMO that is a genetically modified dermal micro-organ. In one embodiment, regulatory elements comprised in a vector of this invention include at least an S/MAR sequence, a CAG promoter, a WPRE sequence and a poly(A) sequence. In another embodiment, regulatory elements comprised in a vector of this invention include at least a CAG promoter and a poly(A) sequence. In yet another embodiment, regulatory element comprised in a vector of this invention include at least an S/MAR sequence, a EF1α promoter, a WPRE sequence and a poly(A) sequence. In still another embodiment, regulatory element comprised in a vector of this invention include at least two S/MAR sequences, a EF1α promoter and a poly(A) sequence. In a further embodiment, regulatory elements comprised in a vector of this invention include at least two different S/MAR sequences and an EF1α promoter, wherein one of the S/MAR sequences is a B globin s/MAR sequence. In certain embodiments, the at least one GMMO of the instant invention demonstrates one or more of sustained in vivo expression levels of EPO, decreased decay rates of EPO, and a prolonged therapeutic effect, e.g., sustained increased and maintains percent hematocrit (FIGS. 7a, 7b, 8a, 8b, 9a, 9b, 10a and 10b).

As used herein, the term “subject” refers to a human subject. A “subject” may also be referred to herein as a “patient”. Subjects may be naïve, e.g., a patient naïve to EPO or IFN. Alternatively, subjects may be previously exposed to a therapeutic polypeptide, e.g., EPO or IFN, for instance by way of erythropoietic stimulating agent (ESA) injection therapy or injection of IFN.

For patients previously treated with ESA injection therapy, administration of a GMMO in place of ESA injections may provide erythropoietin over a sustained time period and prevents a decrease in hemoglobin (Hb) levels or percent hematocrit to basal level. In one embodiment, wherein the patient has been treated with ESA, Hb response refers to a prevention of the decrease of Hb level that would otherwise occur naturally and maintenance of elevated Hb levels, compared with the patient's basal level. In one embodiment, hEPO-GMMO administration prevents a decrease in Hb levels. In this way Hb levels may be maintained within the therapeutic window. In some embodiments of this invention the term “EPODURE” is used in place of EPO GMMO or EPO-Biopump and having all of the same meanings and qualities. Likewise, “Biopump,” “BP”, and “GMMO” are used interchangeably herein.

As used herein, the term “erythropoiesis” refers to the process of red blood cell formation or production. EPO is a required element in the regulation of erythropoiesis, i.e., red blood cell production. The measure of an Hb response is also a measure of red blood cell formation, i.e., erythropoiesis.

In one embodiment, implantation of at least one GMMO provides continuously secreted erythropoietin for at least three months. In another embodiment, EPO is secreted for at least four months. In yet another embodiment, EPO is secreted for at least five months. In still another embodiment, EPO is secreted for at least six months. In another embodiment, EPO is secreted for at least one year. In certain embodiments, the secreted EPO is observed as an increase in serum erythropoietin levels compared with basal levels. In one embodiment, increased serum EPO levels are observed for at least three months. In another embodiment, increased serum EPO levels are observed for at least four months. In yet another embodiment, increased serum EPO levels are observed for at least five months. In still another embodiment, increased serum EPO levels are observed for at least six months. In a further embodiment, increased serum EPO levels are observed for at least one year.

In one embodiment, an at least one GMMO of the instant invention comprises a vector, such as an HDAd or AAV vector, comprising a nucleic acid EPO expression cassette comprising SEQ ID NO: 11 or SEQ ID NO: 13. In some embodiments, such a GMMO demonstrates a decrease in the decay rate of serum EPO levels, in vivo, for at least 100 days post-implantation compared to GMMO formulations lacking an upstream MAR sequence (FIGS. 7a, 7b, 9a, 9b, 10a and 10b).

In certain embodiments of this invention, methods further comprise a step of measuring hemoglobin (Hb) levels in the blood of the subject following said implantation, and wherein the measured hemoglobin levels in the subject are increased and then maintained at about 9-11 g/dl or 9-12 g/dl in at least 50% of the measurements for at least three months or hemoglobin levels are maintained at about 9-11 g/dl or 9-12 g/dl in at least 50% of the measurements for at least three months. In one embodiment, the measured hemoglobin levels are 9-11 g/dl or 9-12 g/dl in at least 50% of the measurements for at least six months.

As used herein, a sustained “Hb response” may also be referred to as sustained “erythropoiesis” having all the qualities and properties of an Hb response.

As used herein, the term “increased Hb levels” refers to an increase in blood Hb levels over basal levels in response to administration of a long-lasting therapeutic formulation of the current invention to a subject in need. As used herein, the term “increased Hb levels” may also be referred to herein as “Hb response”. Administration of a GMMO to a naïve subject may increase Hb levels to a therapeutic level. Administration of a GMMO to a subject previously exposed to EPO may maintain Hb levels at a therapeutic level.

In one embodiment, the Hb response refers to an increase in Hb levels such that Hb levels range between about 9-11 gm/dl, which is the current FDA recommended range. In another embodiment, the Hb levels range between 9.5-12.6 gm/dl. In yet another embodiment, the Hb levels range between 10-12 gm/dl. In still another embodiment, the Hb levels range between 9-13.2 gm/dl. In a further embodiment, the Hb levels range between 8.5-13.8 gm/dl. In another embodiment, the Hb levels range between 8-14.4 gm/dl. In some embodiments, the Hb levels are target therapeutic levels. As used herein, “g” and “gm” are used interchangeably to indicate “gram” or “grams.”

As Hb levels in blood may oscillate slightly from day to day, the range increase in Hb response may in certain situations represent an average increase over any given time period. Measurements made over a given time period may reflect this oscillation. For example, the increased Hb may be maintained for 90% of measurements over any given time period within a target range, as for example presented above. In other words, 90% of measurements made during at least one month or 90% of measurements made over at least six months, or at least one year may be within the Hb target range. Alternatively, Hb levels may be increased or maintained within the targeted range for 80% of measurements over any given time period. Further, Hb levels may be increased or maintained within the targeted range for 70% of measurements over any given time period. Alternatively, Hb levels may be increased or maintained within the targeted range for 60% of measurements over any given time period. Or, Hb levels may be increased or maintained within the targeted range for 50% of measurements over any given time period.

Hb measurements may be made on a regular basis or irregular basis. In certain cases, measurements of blood Hb levels may be made once per week. Alternatively, measurements of blood Hb may be more or less frequent, e.g., twice per week or once every two weeks or once a month. In one embodiment, blood measurements are made once a week. In another embodiment, twice a week. In yet another embodiment, three times a week. In still another embodiment, measurements are made once every two weeks. In a further embodiment, measurements are made once a month. In one embodiment, measurements are made on a regularly scheduled basis. In another embodiment, measurements are made on an as “needed” basis. Measurements may be made more or less frequently, dependent on need.

In some embodiments, increased Hb levels are maintained within a given range for at least 90% of the time that Hb levels are increased. In other embodiments, Hb levels are maintained for at least 80% of the time. In yet other embodiments, Hb levels are maintained for at least 70% of the time. In still other embodiments, Hb levels are maintained for at least 60% of the time. In a further embodiment, Hb levels are maintained for at least 50% of the time. In certain embodiments, methods of this invention providing sustained hemoglobin levels in at least 50% of the measurements avoid non-physiological Hb cycling observed with injections of rhu-EPO.

As used herein, the term “hematocrit” refers to the packed cell volume or erythrocyte volume fraction as a percentage of the concentration of red blood cells in blood. As used herein, increases in Hb levels reflect increases in hematocrit. As used herein, a measurement of “100% hematocrit” refers to a volume of pure packed red blood cells compared to the total volume.

As used herein, the term “nucleic acid” refers to polynucleotide or to oligonucleotides such as deoxyribonucleic acid (DNA), and, where appropriate, ribonucleic acid (RNA) or mimetic thereof.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in DNA sequences, which are caused by point mutations or by induced modifications (including insertion, deletion, and substitution) to enhance the activity, half-life or production of the polypeptides encoded thereby, are also encompassed in the invention. In some embodiments, nucleic acid sequences of this invention include CpG free regions. A major limitation of gene delivery vectors for gene therapy applications is the rapid decline of transgene expression in vivo. Methylation of dinucleotides within the promoter has been observed as a major factor limiting long-lasting gene expression.

In certain embodiments, the efficacy of a regulatory sequence of this invention within a vector of this invention may be analyzed prior to use. The efficacy of expression and method of introducing nucleic acid vectors into a micro-organ can be assessed by standard approaches routinely used in the art as described herein below. In one embodiment, expression and secretion levels of a therapeutic polypeptide by a GMMO are measured in vitro prior to implanting at least one GMMO.

In one embodiment, measurement of in vitro secretion levels provides a guide to determine dose of an at least one GMMO, i.e., the number of GMMOs to implant in a subject. In one embodiment, secretion may be measure using ELISA or any other techniques known in the art.

In one embodiment, the nucleic acid of the at least one GMMO and methods of the instant invention encode a therapeutic polypeptide. In one embodiment, the term “polypeptide” refers to a molecule comprised of amino acid residues joined by peptide (i.e., amide) bonds and includes polypeptides, and proteins. Hence, in one embodiment, the polypeptides of this invention may have single or multiple chains of covalently linked amino acids and may further contain intrachain or interchain linkages comprised of disulfide bonds. In one embodiment, some polypeptides may also form a subunit of a multiunit macromolecular complex. In one embodiment, the polypeptides can be expected to possess conformational preferences and to exhibit a three-dimensional structure. Both the conformational preferences and the three-dimensional structure will usually be defined by the polypeptide's primary (i.e., amino acid) sequence and/or the presence (or absence) of disulfide bonds or other covalent or non-covalent intrachain or interchain interactions.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

As used herein, the term “amino acid” refers to either the D or L stereoisomer form of the amino acid, unless otherwise specifically designated. Also encompassed within the scope of this invention are equivalent proteins or equivalent peptides. “Equivalent proteins” and “equivalent polypeptides” refer to compounds that depart from the linear sequence of the naturally occurring proteins or polypeptides, but which have amino acid substitutions that do not change its biologically activity. These equivalents can differ from the native sequences by the replacement of one or more amino acids with related amino acids, for example, similarly charged amino acids, or the substitution or modification of side chains or functional groups.

The polypeptides, or the DNA sequences encoding same, may be obtained from a variety of natural or unnatural sources, such as a prokaryotic or a eukaryotic cell. In one embodiment, the source cell may be wild type, recombinant, or mutant. In another embodiment, the plurality of polypeptides may be endogenous to microorganisms, such as bacteria, yeast, or fungi, to a virus, to an animal (including mammals, invertebrates, reptiles, birds, and insects) or to a plant cell.

In another embodiment, the polypeptides may be obtained from more specific sources, such as the surface coat of a virion particle, a particular cell lysate, a tissue extract, or they may be restricted to those polypeptides that are expressed on the surface of a cell membrane. The polypeptide of the present invention can be of any size.

As will be appreciated by one skilled in the art, a fragment or derivative of a nucleic acid sequence or gene that encodes for a protein can still function in the same manner as the entire wild type gene or sequence. Likewise, forms of nucleic acid sequences can have variations as compared to wild type sequences, nevertheless encoding the protein of interest, or fragments thereof, retaining wild type function exhibiting the same biological effect, despite these variations.

The polypeptides may include functional fragments. “Functional fragments” are meant to indicate a portion of the polypeptide which is capable of performing one or more of the functions of the polypeptide, even in the absence of the remainder of the polypeptide. In one embodiment, the functional fragment is sufficient to mediate an intermolecular interaction with a target of interest.

According to other embodiments of the present invention, recombinant gene products may be encoded by a polynucleotide having a modified nucleotide sequence, as compared to a corresponding natural polynucleotide.

As described hereinabove, in one embodiment, the at least one GMMO and methods of the present invention provide a therapeutic formulation comprising a vector comprising a nucleic acid sequence encoding a therapeutic polypeptide. In one embodiment, the term “therapeutic” refers to a molecule, which when provided to a subject in need, provides a beneficial effect. In some cases, the molecule is therapeutic in that it functions to replace an absence or diminished presence of such a molecule in a subject. In one embodiment, the therapeutic protein is that of a protein which is absent in a subject, such as in cases of subjects with an endogenous null or mis-sense mutation of a required protein. In other embodiments, the endogenous protein is mutated, and produces a non-functional protein, compensated for by the provision of the functional protein. In other embodiments, expression of a heterologous protein is additive to low endogenous levels, resulting in cumulative enhanced expression of a given protein. In other embodiments, the molecule stimulates a signaling cascade that provides for expression, or secretion, or others of a critical element for cellular or host functioning.

In one embodiment, the term “therapeutic formulation” describes a substance applicable for use in the diagnosis, or in another embodiment, cure, or in another embodiment, mitigation, or in another embodiment, treatment, or in another embodiment, prevention of a disease, disorder, condition or infection. In one embodiment, the “therapeutic formulation” of this invention refers to any substance which affects the structure or function of the target to which it is applied. In one embodiment, a therapeutic micro-organ comprises a genetically modified micro-organ of this invention expressing a therapeutic polypeptide. Thus, in one embodiment, a therapeutic micro-organ comprises a genetically modified dermal micro-organ expressing a therapeutic polypeptide. In one embodiment, a therapeutic micro-organ expresses EPO. In another embodiment, a therapeutic micro-organ expresses IFN.

In another embodiment, the “therapeutic polypeptide” of the present invention is a molecule that alleviates a symptom of a disease or disorder when administered to a subject afflicted thereof.

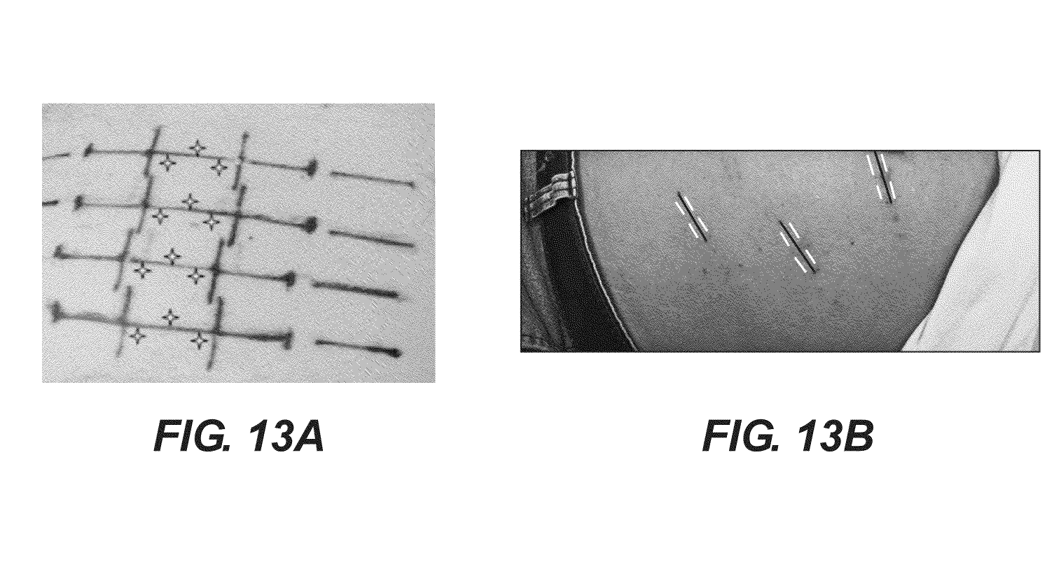

In certain embodiments, methods of this invention include administration of an anti-inflammatory agent, an anti-proliferation agent and/or anti-oxidant agent following the implantation of a GMMO, wherein the target administering location is near or around the implanted GMMO, which in one embodiment is by subcutaneous injection around each GMMO implantation site (FIGS. 13a and 13b). In certain embodiments, administration of an anti-inflammatory agent, an anti-proliferation agent and/or anti-oxidant agent is concurrent with implantation of a GMMO.

The term “anti-inflammatory” agent, as used herein, in one embodiment refers to a substance or treatment that reduces inflammation. Anti-inflammatory may remedy pain by reducing inflammation. The term “anti-proliferation” agent, as used herein, in one embodiment refers to a substance or treatment that partially or fully inhibits cell growth. The term “anti-oxidant”, as used herein, in one embodiment refers to a substance or treatment that inhibits the oxidation of other molecules. Antioxidants are substances that may protect your cells against the effects of free radicals. Free radicals are molecules produced when your body breaks down food, or by environmental exposures like tobacco smoke and radiation. Free radicals can damage cells.

Anti-inflammatory agents, anti-proliferation agents and/or anti-oxidant agents that may be used in methods of this invention include Vitamin C, N-Acetyl Cysteine, Caspase-1 Inhibitor (Z-Wehd-Fmk), Cytosine, Pirfendone, Tempol, Cathepsin B inhibitor (CA-074-OME), Demecolcine, zVAD (pan caspase inhibitor), Minocycline hydrochloride (caspase 1 and 3 inhibitor), tocilizumab (Actemra®) (IL-6 inhibitor), Aspirin (cox inhibitor), MIF antagonist (macrophage migration inhibitory factor), Infliximab (Anti TNF), Mitomycin C, Resveratrol, Hyaluronic Acid and methylprednisolone, and any other anti-inflammatory agents, anti-proliferation agents and/or anti-oxidants that are known in the art.

In one embodiment, an anti-inflammatory agent is methylprednisolone. In another embodiment, an anti-inflammatory agent is triamcinolone acetonide. In still another embodiment, an anti-inflammatory agent is triamcinolone hexacetonide.

In one embodiment, a method of this invention further comprises a step of administering methylprednisolone, e.g., Depo-Medrol®, following a GMMO implanting step, wherein the methylprednisolone is administered by subcutaneous injection around each genetically modified micro-organ implantation site.

In some embodiments, methods of this invention include subcutaneous administration of an anti-inflammatory agent, an anti-proliferation agent and/or an anti-oxidant following the subcutaneous implantation of a therapeutic GMMO, for example an EPO GMMO, wherein the administering is by subcutaneous injection around each genetically modified micro-organ implantation site. In one embodiment, administration includes at least one subcutaneous injection per administration. In another embodiment, administration includes at least two subcutaneous injections per administration. In another embodiment, administration includes at least three subcutaneous injections per administration. In another embodiment, administration is along the entire length of a linearly implanted GMMO.

Methylprednisolone is an anti-inflammatory glucocorticoid. In one embodiment, methylprednisolone is administered in a method of this invention. In some embodiments, methods of this invention include subcutaneous administration of methylprednisolone following the subcutaneous implantation of a therapeutic GMMO, for example an EPO GMMO, wherein the administering is by subcutaneous injection around each genetically modified micro-organ implantation site. In one embodiment, administration includes at least one subcutaneous injection per administration. In another embodiment, administration includes at least two subcutaneous injections per administration. In another embodiment, administration includes at least three subcutaneous injections per administration. In another embodiment, administration is along the entire length of a linearly implanted GMMO.

The duration of use of an anti-inflammatory agent, an anti-proliferation agents and/or anti-oxidant following implantation of a therapeutic GMMO may, in certain embodiments, include a single administration at the time of GMMO implantation. In alternate embodiments, administration of an anti-inflammatory agent, an anti-proliferation agents and/or anti-oxidant following implantation of a therapeutic GMMO is repeated weekly for up to eight-weeks.

In one embodiment, an anti-inflammatory agent, an anti-proliferation agent and/or an anti-oxidant is administered once following GMMO implantation. In another embodiment, an anti-inflammatory agent, an anti-proliferation agent and/or an anti-oxidant administration is weekly. In one embodiment, an anti-inflammatory agent, an anti-proliferation agent and/or an anti-oxidant is administered weekly for up to eight weeks following GMMO implantation. In another embodiment, an anti-inflammatory agent, an anti-proliferation agent and/or an anti-oxidant administration is bi-weekly (every other week), wherein in one embodiment administration continues for up to eight weeks following GMMO implantation. In still another embodiment, an anti-inflammatory agent, an anti-proliferation agent and/or an anti-oxidant administration is semi-weekly (twice weekly), wherein in one embodiment administration continues for up to eight weeks following GMMO implantation. In yet another embodiment, an anti-inflammatory agent, an anti-proliferation agent and/or an anti-oxidant administration is on an “as needed” basis. In a further embodiment, an anti-inflammatory agent, an anti-proliferation agent and/or an anti-oxidant administration is limited to at the time of GMMO implantation. An anti-inflammatory agent, an anti-proliferation agent and/or an anti-oxidant injection may be made following GMMO implantation. Alternatively, an anti-inflammatory agent, an anti-proliferation agent and/or an anti-oxidant injection may be made concurrent with GMMO implantation. In certain embodiments, an anti-inflammatory agent, an anti-proliferation agent and/or an anti-oxidant may be administered both at the time of GMMO implantation and at times following GMMO implantation, including any time after GMMO implantation.

In one embodiment, methylprednisolone is administered once following GMMO implantation. In another embodiment, methylprednisolone administration is weekly for a time period of up to eight weeks. In one embodiment, methylprednisolone is administered weekly for up to eight weeks following GMMO implantation. In another embodiment, methylprednisolone administration is bi-weekly (every other week), wherein in one embodiment administration continues for up to eight weeks following GMMO implantation. In still another embodiment, methylprednisolone administration is semi-weekly (twice weekly), wherein in one embodiment administration continues for up to eight weeks following GMMO implantation. In another embodiment, methylprednisolone is administered on day 45 following implantation. In yet another embodiment, methylprednisolone administration is on an “as needed” basis. In another embodiment, methylprednisolone is administered at the time of GMMO implantation and at day 45 following implantation. In a further embodiment, methylprednisolone administration is limited to at the time of GMMO implantation. Methylprednisolone injections may be made following GMMO implantation. Alternatively, methylprednisolone injection may be made concurrent with GMMO implantation. In certain embodiments, methylprednisolone may be administered both at the time of GMMO implantation and at times following GMMO implantation, including any time after GMMO implantation.

In one embodiment in methods of this invention, wherein implantation of a GMMO is followed by or concurrent with an anti-inflammatory agent, anti-proliferative agent and or an anti-oxidant injections, for instance methylprednisolone, injections are made no more than 1 mm away from a GMMO implantation site. In another embodiment, injection of an anti-inflammatory agent, anti-proliferative agent and or an anti-oxidant is no more than 2 mm away from a GMMO implantation site. In still another embodiment, injection of an anti-inflammatory agent, anti-proliferative agent and or an anti-oxidant is no more than 3 mm away from a GMMO implantation site. In yet another embodiment, injection of an anti-inflammatory agent, anti-proliferative agent and or an anti-oxidant is no more than 4 mm away from a GMMO implantation site. In a further embodiment, injection of an anti-inflammatory agent, anti-proliferative agent and or an anti-oxidant is no more than 5 mm away from a GMMO implantation site. In another embodiment, injection of an anti-inflammatory agent, anti-proliferative agent and or an anti-oxidant is no more than 6 mm away from a GMMO implantation site. In still another embodiment, injection of an anti-inflammatory agent, anti-proliferative agent and or an anti-oxidant is no more than 8 mm away from a GMMO implantation site. In still another embodiment, injection of an anti-inflammatory agent, anti-proliferative agent and or an anti-oxidant is no more than 10 mm away from a GMMO implantation site.

Placement and number of injections of an anti-inflammatory agent, anti-proliferative agent and/or an anti-oxidant may depend on a number of factors including the number and dimensions of the GMMO(s) being implanted. Skilled clinical personnel using their knowledge of the art may choose different administration regimes for different subjects or for the same subject undergoing implantation of GMMOs at different times.

The sum of anti-inflammatory agent, anti-proliferative agent and/or anti-oxidant injections in the area of each GMMO implantation site at any given administration thereof is considered a dose of the agent/GA/MO. For example, the sum of methylprednisolone injections in the area of each GMMO implantation site at any given administration thereof is considered a methylprednisolone dose/GMMO. In one embodiment, a dose of methylprednisolone is about 1-120 mg per GMMO. In one embodiment, a dose of methylprednisolone is about 1-60 mg per GMMO. In one embodiment, a dose of methylprednisolone is about 1-30 mg per GMMO. In another embodiment, a dose of methylprednisolone is about 1-5 mg per GMMO. In yet another embodiment, a dose of methylprednisolone is about 5-10 mg per GMMO. In still another embodiment, a dose of methylprednisolone is about 10-15 mg per GMMO. In a further embodiment, a dose of methylprednisolone is about 15-25 mg per GMMO. In another embodiment, a dose of methylprednisolone is about 25-45 mg per GMMO. In yet another embodiment, a dose of methylprednisolone is about 45-65 mg per GMMO. In still another embodiment, a dose of methylprednisolone is about 65-85 mg per GMMO. In a further embodiment, a dose of methylprednisolone is about 85-105 mg per GMMO. In another embodiment, a dose of methylprednisolone is about 105-120 mg per GMMO. In one embodiment, a dose of methylprednisolone is about 1-12 mg per GMMO. In one embodiment, a dose of 12 mg methylprednisolone per GMMO is administered.

In one embodiment, doses of methylprednisolone do not exceed 120 mg for all GMMO implantation sites in a given patient at a given administration.

In certain embodiments, subcutaneous anti-inflammatory agents, anti-proliferative agents or anti-oxidants can be administered at a therapeutic GMMO implantation site. For example, in one embodiment, subcutaneous administration of an inflammatory agent, an anti-proliferative agent or an anti-oxidant at the site of a therapeutic GMMO implantation, for example an EPO GMMO implantation, may result in a decreased decay rate of the therapeutic polypeptide serum levels. In other words, subcutaneous administration of an inflammatory agent, an anti-proliferative agent or an anti-oxidant at the site of a therapeutic GMMO may result in a sustained increase in serum levels of the therapeutic polypeptide, compared to basal serum levels, over a period of at least three months. In another embodiment, a sustained increase of a therapeutic polypeptide is for a period of at least four months. In still another embodiment, a sustained increase of a therapeutic polypeptide is for a period of at least five months. In yet another embodiment, a sustained increase of a therapeutic polypeptide is for a period of at least six months. In a further embodiment, a sustained increased of a therapeutic polypeptide is for a period of at least one year.

In certain embodiments, subcutaneous administration of methylprednisolone at the site of a therapeutic GMMO implantation, for example an EPO GMMO implantation, may result in a decreased decay rate of the therapeutic polypeptide serum levels. In other words, subcutaneous administration of methylprednisolone at the site of a therapeutic GMMO may result in a sustained increase in serum levels of the therapeutic polypeptide, compared to basal serum levels, over a period of at least three months. In another embodiment, a sustained increase of a therapeutic polypeptide is for a period of at least four months. In still another embodiment, a sustained increase of a therapeutic polypeptide is for a period of at least five months. In yet another embodiment, a sustained increase of a therapeutic polypeptide is for a period of at least six months. In a further embodiment, a sustained increased of a therapeutic polypeptide is for a period of at least one year.

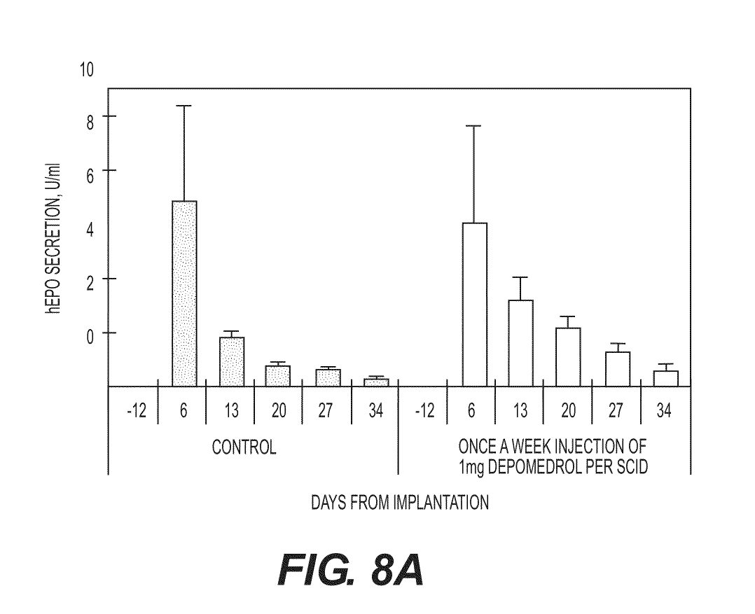

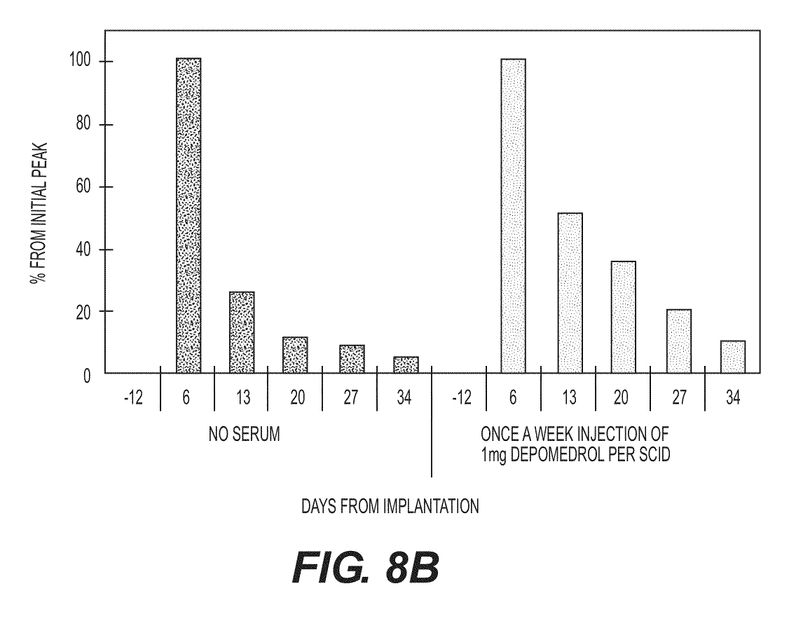

For example, subcutaneous administration of methylprednisolone at the site of therapeutic EPO GMMO implantation resulted in decreased decay rate of EPO serum levels in vivo, compared with levels measured in the absence of methylprednisolone administration (FIGS. 8b, 9b and 10b). By decreasing the decay rate in serum of a therapeutic polypeptide provided by a GMMO of this invention, for instance EPO, the sustained delivery of the therapeutic polypeptide, e.g., EPO, is increased and improved. Methods of implantation of a GMMO that include administration of methylprednisolone effectively providing a longer-lasting therapeutic polypeptide dosage, for example an EPO dose, to a subject in need thereof.

As used herein, the term “decay rate” refers in one embodiment to rate of decrease of a therapeutic polypeptide from peak serum levels. FIGS. 7a, 7b, 8a, 8b, 9a, 9b, 10a and 10b illustrate that a decreased decay rate of a therapeutic polypeptide correlates with sustained delivery of the therapeutic polypeptide to a subject.

In one embodiment, the use of methylprednisolone in methods of this invention decreases the decay rate of a therapeutic polypeptide expressed and secreted by a GMMO. In one embodiment, the use of methylprednisolone in methods of this invention decreases the decay rate of EPO expressed and secreted by an EPO GMMO. In another embodiment, the use of methylprednisolone in methods of this invention decreases the decay rate of IFN expressed and secreted by an IFN GMMO.

In one embodiment, administration of an anti-inflammatory agent, an anti-proliferation agent and/or an anti-oxidant agent at the time of implanting a therapeutic GMMO decreases the decay rate of the therapeutic polypeptide expressed and secreted by the GMMO, thereby providing the therapeutic polypeptide to a subject in need over a sustained time period. In one embodiment, an anti-inflammatory agent, an anti-proliferation agent and/or an anti-oxidant agent is methylprednisolone.

In certain embodiments, methods of this invention further comprise administration of a topical corticosteroid at the GMMO implantation site. Topical corticosteroids of this invention, included high potency, broad potency and low potency corticosteroids. A topical corticosteroid used in the methods of this invention may be in a form or vehicle including a ointment, an optimized ointment, a lotion, a gel, a cream, an emollient base, a foam, an aerosol, a foam aerosol, a shampoo, a solution, a spray, a tape, a petrolatum ointment, an augmented cream, an anhydrous cream, a hydrophobic emollient, an aqueous emollient, or an oil. In one embodiment, a topical corticosteroid used in the methods of this invention comprises a betamethasone dipropionate (Diprolene®), clobetasol propionate (Temovate®, Clobex®, Olux®-E Olux®, Cormax®), halobetasol propionate (Ultravate®), flucinonide (Vanos®), flurandrenolide (Cordran®), diflorasone diacetate (Psorcon®, ApexiCon®), amcinonide (Cyclocort®, Amcort®), betamethason dipropionate (Dipronsone®, Diprolene® AF), halcinonide (Halog®), fluocinonide (Lidex®), diflorasone diacetate (ApexiCon®, Florone®), desoximetasone (Topicort®), triamcinolone acetonide (Kenalog®, Triderm®, Aristocort® HP, Aristocort® A, Aristocort®), betamethasone valerate (Valisone®, Luxiq®, Beta-Val®), fluticasone propionate (Cutivate®), fluocinonide (Lidex®-E), mometasone furoate (Elocon®), fluocinolone acetonide (Synalar®, Capex®, Derma-Smoothe®/FS), mometasone furoate (Elocon®), hydrocortisone valerate (Westcort®), clocortolone pivalate (Cloderm®), prednicarbate (Dermatop®), desonide (DesOwen®, Tridesilon®, Desonate®, LoKara®, Verdeso®), hydrocortisone butyrate (Locoid®, Lipocream®, Cortizone®-10), hydrocortisone probutate (Pandel®), alclometasone dipropionate (Aclovate®), or hydrocortisone (base) (Hytone®, Nutracort®, Texacort®, Cortaid®, Synacort®, Aquinil® HC, Sarnol® HC, Cortizone®-10, Noble, Scalp relief), or any combination thereof. In one embodiment, the at least one topical steroid is a betamethasone valerate. In one embodiment, the at least one topical steroid is a betamethasone valerate in the form of a foam.

In one embodiment, methods of this invention comprise application of an at least one topical corticosteroid for at least two weeks following implantation of an at least one genetically modified micro-organ. In another embodiment, application of an at least one topical corticosteroid is for at least three weeks. In yet another embodiment, application of an at least one topical corticosteroid is for at least four weeks. In still another embodiment, application of an at least one topical corticosteroid is for at least six weeks. In a further embodiment, application of an at least one topical corticosteroid is for at least eight weeks.

As used herein, the term “sustained” refers to an extended period of time. For example an extended period of time, in one embodiment, refers to the amount of time a therapeutic polypeptide of this invention is expressed and secreted from genetically modified micro-organs of this invention. In another embodiment, an extended period of time refers to the amount of time a therapeutic effect is observed following implanting a GMMO of this invention. In yet another embodiment, an extended period of time refers to the extended presence of a therapeutic polypeptide of this invention, in vivo. In one embodiment, a sustained or extended presence of a therapeutic polypeptide is observed as a reduced decay rate of the therapeutic polypeptide, following implantation of a GMMO and administration of methylprednisolone. In another embodiment, a sustained or extended presence of a therapeutic polypeptide is observed as a reduced decay rate of the therapeutic polypeptide, following implantation of a GMMO of this invention without further administration of methylprednisolone. The term “prolonged” may be used interchangeably with “sustained” with all the same meanings and qualities.

In one embodiment, the therapeutic polypeptide is erythropoietin (EPO). “Erythropoietin” or “EPO” as used herein refers to a full length EPO polypeptide from a mammal such as a human as well as a fragment thereof that retains at least one of the biological functions and/or in vivo therapeutic beneficial effects of the full-length EPO. In one embodiment, at least one in vivo therapeutic beneficial effect provided by EPO is increased Hb levels. In another embodiment, at least one in vivo therapeutic beneficial effect provided by EPO is maintenance of Hb levels. In yet another embodiment, at least one in vivo therapeutic beneficial effect provided by EPO is increase and maintenance of Hb levels. In one embodiment, at least one in vivo therapeutic beneficial effect provided by EPO is increased percent hematocrit. In another embodiment, at least one in vivo therapeutic beneficial effect provided by EPO is maintenance of percent hematocrit. In yet another embodiment, at least one in vivo therapeutic beneficial effect provided by EPO is increase and maintenance of percent hematocrit. In one embodiment, at least one in vivo therapeutic beneficial effect provided by EPO is treatment of anemia.

In one embodiment, a method of this invention provides increased serum erythropoietin levels in a human subject over a continuous, sustained period of time the method comprising the steps of: providing at least one genetically modified micro-organ expressing and secreting human erythropoietin, the micro-organ comprising a helper-dependent adenoviral vector comprising a nucleic acid sequence encoding erythropoietin operably linked to an upstream MAR regulatory sequence, and wherein the nucleic acid optionally further comprises at least one or more additional regulatory sequences; determining erythropoietin secretion levels of said at least one genetically modified micro-organ in vitro; implanting the at least one genetically modified micro-organ in the subject at an effective dosage; and measuring erythropoietin levels in the blood serum of the subject, wherein implantation of the at least one genetically modified micro-organ increases the in vivo serum erythropoietin levels over basal levels for at least three months. In one embodiment, said increase is for at least six months. In one embodiment, said nucleic acid comprises SEQ ID NO: 11 or SEQ ID NO: 13 or SEQ ID NO: 15 or SEQ ID NO: 17.

In one embodiment, a method of this invention comprises treating anemia in a human subject in need over a sustained time period comprising the steps of: providing at least one genetically modified micro-organ that provides a sustained delivery of human erythropoietin, the micro-organ comprising a vector, such as an HDAd or AAV vector, comprising a nucleic acid sequence encoding erythropoietin operably linked to one or more regulatory sequences; determining erythropoietin secretion levels of said at least one GMMO in vitro; implanting said at least one GMMO in said human subject at an effective dosage; and measuring hemoglobin levels in the blood of said subject; wherein the measured hemoglobin levels in said subject are maintained at about 9-11 g/dl in at least 50% of the measurements for at least one month. In one embodiment, the increase and/or maintained Hb levels are for at least three months. In another embodiment, the increased and/or maintained Hb levels are for at least six months. In one embodiment, the nucleic acid encoding erythropoietin operably linked to one or more regulatory sequences comprises SEQ ID NO: 11 or SEQ ID NO: 13 or SEQ ID NO: 15 or SEQ ID NO: 17.

Herein, “treatment” refers to both therapeutic treatment and prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of delaying the onset of reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of a particular disease, while in one embodiment; “secondary” refers to a symptom that is derived from or consequent to a primary cause. In one embodiment, the compounds for use in the present invention treat primary or secondary symptoms or secondary complications related to the disease. In another embodiment, “symptoms” may be any manifestation of a disease or pathological condition.

In one embodiment, the therapeutic nucleic acids may encode or the therapeutic polypeptides may be cytokines, such as erythropoietin.

In another embodiment, the therapeutic nucleic acid may encode or the therapeutic polypeptide may be an enzyme, such as one involved in glycogen storage or breakdown. In another embodiment, the therapeutic protein comprises a transporter, such as an ion transporter, for example CFTR, or a glucose transporter, or other transporters whose deficiency, or inappropriate expression, results in a variety of diseases.

According to this aspect of the invention, the GMMO and methods of this invention are for treatment of prevention of or therapeutic intervention in disease. In one embodiment, the disease for which the subject is thus treated may comprise, but is not limited to: renal failure, chronic renal failure, chemotherapy induced anemia, anemia as a result of HIV treatments, microangiopathic haemolytic anemia, anemia as a result of prematurity, an inflammatory condition including rheumatoid arthritis, an infection, anemia associated with cancers including multiple myeloma and non-Hodgkin lymphoma, hematopoietic stem cell disorders, anemia associated with myelodysplastic syndrome (MDS), sickle cell anemia or thalassemia, or a need of accelerated erythroid repopulation after bone marrow transplantation, hepatitis, hepatitis B, hepatitis C or hepatitis D, or any combination thereof.

It is to be understood that any disease whereby expression of a particular protein, provision of a therapeutic protein, which can be accomplished via the formulations of this invention and according to the methods of this invention, is to be considered as part of this invention.

As used herein, “treatment” or “treating” of anemia refers to increasing the amount of oxygen that a subject's blood can carry. This may be done by raising the red blood cell count (percent hematocrit) and/or hemoglobin level. Hemoglobin is the iron-rich protein in red blood cells that carries oxygen to the body.

As used herein, “treatment” or “treating” of hepatitis refers to reducing the hepatitis virus, for example, reducing hepatitis B virus or reducing hepatitis C virus or reducing hepatitis D virus or reducing a combination of these viruses thereof. In one embodiment, treatment results in reduction of viral load, e.g., Hepatitis C RNA reduction or Hepatitis B DNA reduction. Reduction of virus number or viral load may be assessed by testing for loss of viral DNA, e.g., loss of Hepatitis B viral DNA. Alternatively, loss of virus number or viral load may be assessed by loss of specific viral antigens such as Hepatitis B “e” antigen (HBeAg) or Hepatitis B “surface” antigen (HBsAg).

In one embodiment, the GMMO and methods of the instant invention comprise a nucleic acid sequence operably linked to one or more regulatory sequences. In one embodiment, a nucleic acid molecule introduced into a cell of a micro-organ is in a form suitable for expression in the cell of the gene product encoded by the nucleic acid. Accordingly, in one embodiment, the nucleic acid molecule includes coding and regulatory sequences required for transcription of a gene (or portion thereof). When the gene product is a protein, the nucleic acid molecule includes coding and regulatory sequences required for translation of the nucleic acid molecule, include promoters, enhancers, polyadenylation signals, sequences necessary for transport of an encoded protein, for example N-terminal signal sequences for transport of proteins or peptides to the surface of the cell or secretion, in one embodiment.

In one embodiment, the GMMO and methods of the instant invention increase the levels of a therapeutic polypeptide in a subject in need following implantation of an at least one GMMO of this invention. In one embodiment, the therapeutic polypeptide is EPO. In another embodiment, the therapeutic polypeptide is IFN. In one embodiment, following implantation of an at least one GMMO, the increase is by at least 5% over basal levels. In another embodiment, the levels of a therapeutic polypeptide are increased by at least 7%, in another embodiment, by at least 10%, in another embodiment, by at least 15%, in another embodiment, by at least 20%, in another embodiment, by at least 25%, in another embodiment, by at least 30%, in another embodiment, by at least 40%, in another embodiment, by at least 50%, in another embodiment, by at least 60%, in another embodiment, by at least 75%, in another embodiment, by at least 100%, in another embodiment, by at least 125%, in another embodiment, by at least 150% over basal levels, in another embodiment, by at least 200% over basal levels. In still another embodiment, the formulations and methods of the instant invention increase the level of a therapeutic polypeptide upon implantation of an at least one GMMO, wherein the level of the therapeutic polypeptide then returns to basal or near basal levels. In one embodiment, the return to basal or near basal levels occurs within one month of administration of the therapeutic peptide or nucleic acid.

In one embodiment, implantation of an at least one GMMO of this invention provides a “sustained” or “long-lasting” delivery of the therapeutic polypeptide. In one embodiment, implantation of an at least one GMMO of this invention comprising at least one MAR regulatory nucleic acid sequences provides a sustained or long-lasting delivery of the therapeutic polypeptide compared with implantation of a similar GMMO lacking at least one MAR regulatory nucleic acid sequence. In one embodiment, expression levels of a therapeutic polypeptide are increased over basal levels for at least one month, or in another embodiment, for at least three months, while in a further embodiment, for at least six months.

In one embodiment, the methods of this invention administering methylprednisolone at the time of or following implantation of at least one GMMO, decrease the decay rate of a therapeutic polypeptide of this invention in comparison with an implantation regime of a therapeutic GMMO that does not include administering methylprednisolone. In one embodiment the effect of methylprednisolone administration and implantation of a GMMO comprising at least one MAR regulatory nucleic acid sequence on therapeutic polypeptide decay rate are additive. In one embodiment, the therapeutic polypeptide is EPO. In one embodiment, the therapeutic polypeptide is IFN. In one embodiment the decreased decay rate is for at least one week. In another embodiment, the decreased decay rate is for at least one month. In still another embodiment, the decreased decay rate is for at least two months. In yet another embodiment, the decreased decay rate is for at least three months. In a further embodiment, the decreased decay rate is for at least four months. In another embodiment, the decreased decay rate is for at least five months. In still another embodiment, the decreased decay rate is for at least six months.

In one embodiment, expression of a therapeutic polypeptide via the formulation of the present invention is increased compared to “basal levels”, which in one embodiment, are levels of the gene expressed in hosts or cell culture that have not been genetically modified.

In one embodiment, the formulations and methods of the instant invention increase the levels of a functional marker, which in one embodiment, is percent hematocrit.

In another embodiment, the GMMO of the present invention is “long-lasting”, which in one embodiment refers to a formulation that can increase secretion, expression, production, circulation or persistence of a target molecule of the therapeutic polypeptide or nucleic acid. For example, when the therapeutic polypeptide is EPO, a target molecule may be Hb. Accordingly, in one embodiment, use of a GMMO of the present invention may increase production, circulation or persistence of Hb in a subject.

In yet another embodiment of the invention, the GMMO of the present invention is “long-lasting”, which refers to a GMMO that can increase secretion, expression, production, circulation or persistence of a functional marker. In one embodiment, the functional marker is hematocrit. In another embodiment, the functional marker is Hb. In yet another embodiment, the levels of a functional marker, for example hematocrit or Hb, are increased for at least 2 weeks, in another embodiment, for at least 3 weeks, in another embodiment, for at least 4 weeks, in another embodiment, for at least 5 weeks, in another embodiment, for at least 6 weeks, in another embodiment, for at least 8 weeks, in another embodiment, for at least 2 months, in another embodiment, for at least 3 months in another embodiment, for at least 4 months, in another embodiment, for at least 5 months, in another embodiment, for at least 7 months, in another embodiment, for at least 8 months, in another embodiment, for at least 9 months, in another embodiment, for at least 10 months, in another embodiment, for at least 11 months, or, in another embodiment, for at least 1 year. In one embodiment, increase of the functional marker, e.g., Hb, over basal levels is reflected in 90% of measurements made during any time period. In still another embodiment, the increase is reflected in 80% of the measurements made during any time period, e.g., one month, six months or a year. In a further embodiment, the increase is reflected in 70% of the measurements made during any time period, e.g., one month, six months or a year. In another embodiment, the increase is reflected in 60% of the measurements made during any time period, e.g., one month, six month or a year. In a yet another embodiment, the increase is reflected in 50% of the measurements made during any time period, e.g., one month, six months or a year.

In one embodiment, the nucleic acid sequence encoding a therapeutic polypeptide is optimized for increased levels of therapeutic polypeptide expression, or, in another embodiment, for increased duration of therapeutic polypeptide, or, in another embodiment, a combination thereof. In one embodiment, nucleic acid sequences of this invention include CpG free sequences. In one embodiment, regulatory nucleic acid sequences are CpG free.

In one embodiment, the term “optimized” refers to a desired change, which, in one embodiment, is a change in gene expression and, in another embodiment, in protein expression. In one embodiment, optimized gene expression is optimized regulation of gene expression. In another embodiment, optimized gene expression is an increase in gene expression. According to this aspect and in one embodiment, a 2-fold increase in gene expression compared to wild-type is contemplated. In another embodiment, there is a 4-fold increase in gene expression. In yet another embodiment, there is a 6-fold increase in gene expression. In still another embodiment, there is an 8-fold increase in gene expression. In a further embodiment, there is a 10-fold increase in gene expression.

In another embodiment, optimized gene expression may be an increase in gene expression under particular environmental conditions. In another embodiment, optimized gene expression may comprise a decrease in gene expression, which, in one embodiment, may be only under particular environmental conditions.

In another embodiment, optimized gene expression is an increased duration of gene expression.

In one embodiment, a gene is optimized for expression in homo sapien cells. In another embodiment, a gene is optimized for expression in micro-organs. In yet another embodiment, a gene is optimized for expression in dermal cells. In still another embodiment, optimizing a gene expression entails adding sequence elements to flanking regions of a gene and/or elsewhere in the expression vector. Sequence elements that may be added for optimizing gene expression include, for example, scaffold/matrix-attached regions (S/MAR), specialized chromatin structures (SCS) and woodchuck hepatitis post-transcriptional regulatory elements (WPRE).

In one embodiment, this invention provides a GMMO as described hereinabove in which the therapeutic polypeptide is EPO. In another embodiment, this invention provides a GMMO that provides a sustained delivery of EPO, said micro-organ comprising a vector comprising a nucleic acid sequence operably linked to one or more regulatory sequences, wherein said nucleic acid sequence encodes EPO and whereby said formulation increases EPO levels or an EPO target such as hematocrit or Hb by more than 5% over basal levels and said increased levels persist for greater than one month. In yet another embodiment, the invention provides a method of providing a GMMO to a subject in need in which the therapeutic polypeptide is EPO or wherein the therapeutic nucleic acid encodes EPO. In still another embodiment, the invention provides a method of providing EPO to a subject in need over a sustained time period.

In yet another embodiment, this invention provides a method of inducing formation of new blood cells in a subject in need over a sustained period comprising: providing one or more GMMOs, said micro-organs comprising a vector comprising a nucleic acid sequence operably linked to one or more regulatory sequences; and implanting said GMMO in said subject, wherein said nucleic acid sequence encodes EPO and whereby EPO levels are increased by more than 5% over basal levels and said increased EPO levels persist for greater than one month.

The identification, cloning, and expression of genes encoding EPO are described in U.S. Pat. Nos. 5,756,349; 5,955,422; 5,618,698; 5,547,933; 5,621,080; 5,441,868; and 4,703,008, which are incorporated herein by reference. A description of the purification of recombinant EPO from cell medium that supported the growth of mammalian cells containing recombinant EPO plasmids for example, are included in U.S. Pat. No. 4,667,016 to Lai et al, which is incorporated herein by reference.

In one embodiment, a subject suffering from chronic renal failure is suffering from chronic kidney disease (CKD). In another embodiment, a subject suffering from chronic renal failure is suffering from end stage renal disease (ESRD). EPO may be used in the treatment of anemia as a result of renal failure including CKD and ESRD; anemia associated with HIV infection in zidovudine (AZT) treated patients; anemia associated with cancer chemotherapy; microangiopathic haemolytic anemia that may be a secondary to mechanical valve haemolysis; anemia of prematurity; anemia as a result of rheumatoid arthritis and other inflammatory conditions; and anemia associated with cancer including multiple myeloma and non-Hodgkin lymphoma. In addition, administration of EPO may benefit subjects prior to scheduled surgery, subjects suffering from hematopoietic stem cell disorders, anemia associated with myelodysplastic syndrome (MDS), subjects in need of acceleration of erythroid repopulation after bone marrow transplantation, or subjects in need of induction of fetal Hb synthesis as a result of sickle cell anemia and thalassemia.

Administration by injection of rHu-EPO (recombinant human-EPO) has become routine in the treatment of anemia secondary to renal insufficiency, where doses of 50-150 U/kg given three times per week are used to restore hematocrit and eliminate transfusion dependency. This calculates to an average daily dosage of 21.4-64.3 EPO U/kg/day to a subject being treated. It has been observed that during rHu-EPO injection treatment in hemodialysis patients, Hb levels often rise and fall in non-physiological undulations or hemoglobin cycles. In one embodiment, methods of this invention provide an effective EPO dosage of 10-150 U erythropoietin/Kg body weight of the subject/day. In one embodiment, methods of this invention provide an effective EPO dosage of 18-150 U erythropoietin/Kg body weight of the subject/day. In another embodiment the effective dosage is 18-30 U erythropoietin/Kg body weight of the subject/day. In yet another embodiment, the effective dosage is 30-50 U erythropoietin/Kg body weight of the subject/day. In still another embodiment the effective dosage is 50-65 U erythropoietin/Kg body weight of the subject/day. In a further embodiment, the effective dosage is determined based on: the subject's weight; the subject's historical hemoglobin levels; and the average amount of erythropoietin administered to the subject in the one month prior the implanting of an at least one GMMO step.

In one embodiment, the EPO of the compositions and for use in the methods of the present invention are fully glycosylated, while in another embodiment, they comprise some glycosylated residues, while in another embodiment, they are not glycosylated.

In one embodiment, the EPO gene may be a wild-type EPO gene, while in another embodiment, the EPO gene may be modified. In one embodiment, the modified EPO gene may be optimized.

In one embodiment, the EPO gene has a nucleic acid sequence that corresponds to that set forth in Genbank Accession Nos: X02158; AF202312; AF202311; AF202309; AF202310; AF053356; AF202306; AF202307; or AF202308 or encodes a protein sequence that corresponds to that set forth in Genbank Accession Nos: CAA26095; AAF23134; AAF17572; AAF23133; AAC78791; or AAF23132. In another embodiment, the EPO precursor gene has a nucleic acid sequence that corresponds to that set forth in Genbank Accession Nos: NM_{—}000799; M11319; BC093628; or BC111937 or encodes a protein sequence that corresponds to that set forth in Genbank Accession Nos: NP_{—}000790; AAA52400; AAH93628; or AAI11938. In another embodiment, the EPO gene has a nucleic acid sequence as presented in SEQ ID NO: 1, while in another embodiment, the EPO gene has an amino acid sequence as presented in SEQ ID NO: 3. In another embodiment, the EPO gene has a nucleic acid as presented in SEQ ID NO: 2. In one embodiment, the nucleic acid encoding EPO operably linked to one or more regulatory sequences has a nucleic acid sequence as presented in SEQ ID NO: 11. In another embodiment, the nucleic acid encoding EPO operably linked to one or more regulatory sequences has a nucleic acid sequence that is homologous to that presented in SEQ ID NO: 11. In one embodiment, the nucleic acid encoding EPO operably linked to one or more regulatory sequences has a nucleic acid sequence as presented in SEQ ID NO: 13. In another embodiment, the nucleic acid encoding EPO operably linked to one or more regulatory sequences has a nucleic acid sequence that is homologous to that presented in SEQ ID NO: 13. In one embodiment, the nucleic acid encoding EPO operably linked to one or more regulatory sequences has a nucleic acid sequence as presented in SEQ ID NO: 15. In another embodiment, the nucleic acid encoding EPO operably linked to one or more regulatory sequences has a nucleic acid sequence that is homologous to that presented in SEQ ID NO: 15. In one embodiment, the nucleic acid encoding EPO operably linked to one or more regulatory sequences has a nucleic acid sequence as presented in SEQ ID NO: 17. In another embodiment, the nucleic acid encoding EPO operably linked to one or more regulatory sequences has a nucleic acid sequence that is homologous to that presented in SEQ ID NO: 17.

In one embodiment, the vector used for genetic modification has a nucleic acid sequence as presented in SEQ ID NO: 12. In one embodiment, the vector used for genetic modification has a nucleic acid sequence homologous to that presented in SEQ ID NO: 12. In another embodiment, the vector used for genetic modification has a nucleic acid sequence as presented in SEQ ID NO: 10. In one embodiment, the vector used for genetic modification has a nucleic acid sequence homologous to that presented in SEQ ID NO: 10. In another embodiment, the vector used for genetic modification has a nucleic acid sequence as presented in SEQ ID NO: 16. In one embodiment, the vector used for genetic modification has a nucleic acid sequence homologous to that presented in SEQ ID NO: 16. In another embodiment, the vector used for genetic modification has a nucleic acid sequence as presented in SEQ ID NO: 14. In one embodiment, the vector used for genetic modification has a nucleic acid sequence homologous to that presented in SEQ ID NO: 14. In another embodiment, the vector used for genetic modification has a nucleic acid sequence as presented in SEQ ID NO: 26. In one embodiment, the vector used for genetic modification has a nucleic acid sequence homologous to that presented in SEQ ID NO: 26. In another embodiment, the vector used for genetic modification has a nucleic acid sequence as presented in SEQ ID NO: 27. In one embodiment, the vector used for genetic modification has a nucleic acid sequence homologous to that presented in SEQ ID NO: 27.

In one embodiment, the GMMO of the present invention may be used to treat a subject having anemia. In one embodiment, anemia is defined as “a pathologic deficiency in the amount of oxygen-carrying Hb in the red blood cells.” Symptoms of anemia include fatigue, diminished ability to perform daily functions, impaired cognitive function, headache, dizziness, chest pain and shortness of breath, nausea, depression, pain, or a combination thereof. In one embodiment, anemia is associated with a poorer prognosis and increased mortality. In one embodiment, the EPO GMMO of the present invention may be used to treat anemia, wherein a subject is treated when one or more symptoms of anemia are alleviated.

Anemia is often a consequence of renal failure due to decreased production of EPO from the kidney. In another embodiment, anemia is caused by lowered red blood cell (erythroid) production by bone marrow due to cancer infiltration, lymphoma or leukemia including non-Hodgkin's lymphoma, multiple myeloma, chemotherapy, mechanical valve haemolysis, prematurity, rheumatoid arthritis, inflammatory conditions, hematopoietic disorders, anemia associated with myelodysplastic syndrome (MDS), sickle cell anemia, thalassemia or marrow replacement. Other causes of anemia comprise, blood loss due to excessive bleeding such as hemorrhages or abnormal menstrual bleeding; cancer therapies such as surgery, radiotherapy, chemotherapy, treatment of AIDs patients with Zidovudine (AZT), immunotherapy, or a combination thereof; infiltration or replacement of cancerous bone marrow; increased hemolysis, which in one embodiment is breakdown or destruction of red blood cells; low levels of EPO, or a combination thereof. In one embodiment, anemia refers to Fanconi anemia, which in one embodiment is an inherited anemia that leads to bone marrow failure (aplastic anemia) and often to acute myelogenous leukemia (AML). In another embodiment, anemia refers to Diamond Blackfan anemia, normocytic anemia, aplastic anemia, iron-deficiency anemia, vitamin deficiency anemia, Sideroblastic Anemia, Paroxysmal Nocturnal Hburia, Anemia of Chronic Disease, Anemia in Kidney Disease and Dialysis, or a combination thereof.

In certain embodiments a method of this invention comprising a step of implanting an EPO GMMO is used to treat a subject suffering from renal failure, chronic renal failure, chemotherapy induced anemia, anemia as a result of HIV treatments, microangiopathic haemolytic anemia, anemia as a result of prematurity, an inflammatory condition including rheumatoid arthritis, an infection, anemia associated with cancers including multiple myeloma and non-Hodgkin lymphoma, hematopoietic stem cell disorders, anemia associated with myelodysplastic syndrome (MDS), sickle cell anemia or thalassemia; or a subject in need of accelerated erythroid repopulation after bone marrow transplantation; or any combination thereof. In one embodiment, the subject suffering from chronic renal failure is suffering from chronic kidney disease (CKD) or end stage renal disease (ESRD). In one embodiment, a subject is a human subject.

In yet another embodiment, the long-lasting EPO GMMO of the instant invention is used for increasing Hb level in a subject in need. A subject in need of increased Hb may, for instance, be a subject prior to major surgery.

It is to be understood that the GMMO and methods of this invention may be used to treat anemia, regardless of the cause of anemia and whether or not the cause of anemia is known. In one embodiment, the GMMO and methods thereof provide an effective EPO therapy. By the term “effective EPO therapy” it is meant a level of EPO sufficient to bring the Hb level in a patient within the therapeutic window. In one embodiment, “effective EPO therapy” refers to an increase in erythropoiesis in a subject in need. In one embodiment, “effective EPO therapy” refers to prevention of a decrease of erythropoiesis in a subject in need. As used herein, the term “effective EPO therapy” may also be referred to herein as an “effective dosage” or “effective dose” of EPO. In one embodiment, “effective EPO therapy” may also refer to increasing, or increasing and maintaining, or maintaining Hb at therapeutic levels in a subject. In another embodiment, “effective EPO therapy” may also refer to increasing, or increasing and maintaining, or maintaining hematocrit at therapeutic levels in a subject.

In one embodiment of the invention, effective EPO therapy is achieved by implanting at least one (1) EPO GMMO into a patient. In another embodiment, effective EPO therapy is achieved by implanting at least two (2) EPO GMMOs into a patient. In one embodiment, effective EPO therapy is achieved by implanting at least three (3) EPO GMMOs into a patient. In one embodiment, effective EPO therapy is achieved by implanting at least four (4) EPO GMMOs into a patient. In one embodiment, effective EPO therapy is achieved by implanting at least five (5) EPO GMMOs into a patient. In one embodiment, effective EPO therapy is achieved by implanting at least six (6) EPO GMMOs into a patient. In one embodiment, effective EPO therapy is achieved by implanting greater than six (6) EPO GMMOs into a patient.

As used herein, the term “therapeutic window” means the desired level of Hb in a subject in need. In one embodiment, the therapeutic window refers to an Hb concentration within the range of 10 gm/dl to 12 gm/dl. In another embodiment, the therapeutic window refers to an Hb concentration within the range of 9-11 gm/dl. In yet another embodiment, the Hb concentration is within the range of 9.5-12.6 gm/dl. In still another embodiment, the Hb concentration is within the range of 9-13.2 gm/dl. In a further embodiment, the Hb concentration is within the range of 8.5-13.8 gm/dl. In another embodiment, the Hb concentration is within the range of 8-14.4 gm/dl. As used herein, the term “therapeutic window” may also be referred to as “therapeutic levels” or “therapeutic Hb levels”. In one embodiment of the invention, an effective EPO therapy brings the Hb (“Hb”) level in a patient within the therapeutic window. In one embodiment, an increase of blood Hb levels above 11.5 g/dl for four consecutive weekly measurements, may be considered outside of the therapeutic window. In another embodiment, an increase of blood Hb levels above 12.0 g/dl for four consecutive weekly measurements, may be considered outside of the therapeutic window. In an effort to avoid an increase of blood Hb levels outside of the therapeutic window, methods of implantation of a long-lasting EPO formulation may be directed to avoid a resultant elevation of serum EPO above the upper limit of the normal physiological range, defined as a level exceeding 200 mU/ml.

As used herein, the terms “effective dosage” or “effective dose” refers to the effective amount of a therapeutic polypeptide expressed from an at least one GMMO per day, which provides a therapeutic effect. For example, an effective dose of a GMMO expressing EPO, may in one embodiment be the total secretion per day from one or more -EPO GMMOs that maintains, or increases and maintains, the Hb within the therapeutic window for that patient.

As used herein, units for EPO are the accepted International units and are referred to herein using the symbol “U” or “IU”.

In one embodiment, dosage for an effective EPO therapy is between 18-150 IU/kg bodyweight of a patient/day. In another embodiment, effective EPO therapy is between 10-150 IU/kg bodyweight of a patient/day. In one embodiment, effective EPO therapy is between 10-20 IU/kg bodyweight of a patient/day. In one embodiment, dosage for an effective EPO therapy is between 20-40 IU/kg bodyweight of a patient/day. In one embodiment, dosage for an effective EPO therapy is between 40-60 IU/kg bodyweight of a patient/day. In one embodiment, dosage for an effective EPO therapy is between 60-80 IU/kg bodyweight of a patient/day. In one embodiment, dosage for an effective EPO therapy is between 80-100 IU/kg bodyweight of a patient/day. In one embodiment, dosage for an effective EPO therapy is between 100-120 IU/kg bodyweight of a patient/day. In one embodiment, dosage for an effective EPO therapy is between 120-150 IU/kg bodyweight of a patient/day.

In another embodiment, dosage for an effective EPO therapy is between 18-25 IU/kg bodyweight/day (low dose). In yet another embodiment, dosage for an effective EPO therapy is between 18-30 IU/kg bodyweight/day. In one embodiment, dosage for an effective EPO therapy is between 35-45 IU/kg bodyweight/day. In still another embodiment, dosage for an effective EPO therapy is between 30-50 IU/kg bodyweight/day. In one embodiment, dosage for an effective EPO therapy is between 55-65 IU/kg bodyweight/day. In a further embodiment, dosage for an effective EPO therapy is between 50-65 IU/kg bodyweight/day.

In one embodiment, dosage for an effective EPO therapy is 20 IU/kg bodyweight of a patient/day. In one embodiment, dosage for an effective EPO therapy is 40 IU/kg bodyweight of a patient/day. In one embodiment, dosage for an effective EPO therapy is 60 IU/kg bodyweight of a patient/day. In one embodiment, dosage for an effective EPO therapy is 80 IU/kg bodyweight of a patient/day. In one embodiment, dosage for an effective EPO therapy is 100 IU/kg bodyweight of a patient/day. In one embodiment, dosage for an effective EPO therapy is 120 IU/kg bodyweight of a patient/day. In one embodiment, dosage for an effective EPO therapy is 150 IU/kg bodyweight of a patient/day. In one embodiment, dosage is not to exceed about 65 IU/kg bodyweight/day.

In alternative embodiments, effective doses may be tailored to each subject individually, taking into account the patient's weight, historical Hb levels and average EPO dose previously administered by ESA injections. The average EPO dose previously administered may be calculated from the time period of one month prior to a method of implantation. Alternatively, the average EPO dosage previously administered may be calculated from a time period greater or less than at least on month. Dosage may be based on the amount of EPO administered during the at least one month prior to implantation, wherein the dosage administered is normalized to a daily basis. In some circumstances, the dosage may be based on the average amount of EPO administered during at least a two month time period prior to implantation. In certain circumstances, the dosage may be based on the average amount of EPO administered during at least a three month time period prior to implantation. In other circumstances, the dosage may be based on the average amount of EPO administered during at least a six month time period prior to implantation. For example, if a subject previously received three injections per week totaling 150 U/kg/week, a tailored dosage may include implantation of at least one GMMO producing approximately 20 U/Kg/day.

In one embodiment, the dosage matches the amount a subject previously received, normalized to a daily basis. In another embodiment, the dosage is reduced by up to 25% of the amount a subject previously received, normalized to a daily basis. In yet another embodiment, the dosage is increased by up to 25% of the amount a subject previously received, normalized to a daily basis. In a further embodiment, the dosage is reduced by up to 50% of the amount a subject previously received, normalized to a daily basis. In yet another embodiment, the dosage is increased by up to 50% of the amount a subject previously received, normalized to a daily basis.

In one embodiment, response to implantation of an at least one EPO GMMO is sustained elevation of Hb levels for at least one month. In one embodiment, response to implantation of an at least one EPO GMMO is sustained elevation of Hb levels for at least two months. In one embodiment, response to implantation of an at least one EPO GMMO is sustained elevation of Hb levels for at least three months. In one embodiment, response to implantation of an at least one EPO GMMO is sustained elevation of Hb levels for at least four months. In one embodiment, response to implantation of an at least one EPO GMMO is sustained elevation of Hb levels for at least five months. In one embodiment, response to implantation of an at least one EPO GMMO is sustained elevation of Hb levels for at least six months. In one embodiment, response to implantation of an at least one EPO GMMO is sustained elevation of Hb levels for greater than six months. In one embodiment, response to implantation of an at least one EPO GMMO is sustained elevation of Hb levels for greater than nine months. In one embodiment, response to implantation of an at least one EPO GMMO is sustained elevation of Hb levels for greater than one year.

It may be that the response to implantation of an at least one EPO GMMO does not sustain elevation of Hb or provide sufficient elevation to bring the Hb to the target window. In such a case, additional EPO GMMOs may be implanted in the subject. For example, if following implantation of at least one EPO GMMO the blood Hb level decreases by about 1 g/dl or more per week, or per two weeks or per month, compared to the baseline Hb average during a period of at least one month prior to implantation, additional EPO GMMOs may be implanted. Alternatively, if following implantation of at least one EPO GMMO the blood Hb level decreases by about 1 g/dl or more per week, or per two weeks or per month, compared to an initial increased average Hb following implantation, additional EPO GMMOs may be implanted. In one embodiment, additional EPO GMMOs will target an increase of up to 25% over the initial EPO dose. In another embodiment, additional EPO GMMOs will target an increase of up to 50% over the initial EPO dose.

Treatment by implanting long-lasting EPO formulations, e.g., EPO GMMOs expressing and secreting EPO, aims to supply continuous production and delivery of EPO to patients in need. Patients in need may include those suffering from anemia and/or those in need of increased Hb. It has been reported that anemic subjects or those in need of increased Hb may benefit from treatment with a more physiological EPO treatment rather than non-physiological bolus injections [Fishbane, S., Hemoglobin cycling in hemodialysis patients treated with recombinant human erythropoietin. Kidney International, vol. 68, 2005, pp. 1337-1343; Fishbane, S., Recombinant Human EPO: Has Treatment Reached its Full Potential, Seminars in Dialysis, Vol 19, No 1, 2006, pp. 1-4]. Implantation of autologous EPO GMMOs, expressing and secreting EPO, back into a patient, wherein the autologous tissue remains localized and supplies sustained treatment, may provide a benefit in comparisons to treatment with bolus injections, which requires patient compliance and may result in non-physiological, regular or irregular peaks and valleys of serum EPO with each injection. Further, implantation of a GMMO providing sustained delivery of a therapeutic polypeptide may provide a benefit in that a dose may be down regulated or therapy terminated by ablation or removal of the GMMO. This is in comparison with gene therapy relying on injecting viral vectors or cells comprising a viral vector, as the location of an implanted GMMO in known and therefore the GMMO may be inactivated or removed with precision.

An advantage of this method is that if the delivered dose of EPO is too high, or if the treatment needs to be terminated for any reason, one or more of the implanted GMMOs may be excised (or even potentially ablated in situ) in order to stop the production and delivery of the EPO therapy. In one embodiment, if the blood level of Hb is greater than 11.5 g/dl for more than two weeks, then at least one EPO GMMO may be removed or inactivated to reduce the EPO dose by about 25%. In another embodiment, if the blood level of Hb is greater than 12.0 g/dl for more than two weeks, then at least one EPO GMMO may be removed or inactivated to reduce the EPO dose by about 25%.

In one embodiment, the formulations and method of the present invention may be administered with other treatments that are effective in treating anemia. In one embodiment, other treatments include iron supplements, vitamin B12 supplements, additional sources of EPO, androgens, growth factors such as G-CSF, or a combination thereof. In another embodiment, the formulations and method of the present invention may be administered in conjunction with other treatments such as blood and marrow stem cell transplants.

In one embodiment, this invention provides a therapeutic GMMO as described hereinabove in which the therapeutic polypeptide is an interferon (IFN), which in some embodiments, is IFN alpha, IFN alpha 2b, IFN beta, IFN lambda, or IFN gamma from a human or another mammal. In some embodiments the IFN polypeptide is a functional fragment of the full length IFN that retains at least one of the biological functions of full length IFN and/or at least one of the in vivo therapeutic beneficial effects of full length IFN. In one embodiment, an at least one GMMO of this invention comprises a vector, such as an HDAd or AAV vector, comprising a nucleic acid sequence encoding an IFN operably linked to an upstream MAR regulatory sequence, and wherein said nucleic acid further comprises at least one or more additional regulatory sequences, and wherein the at least one genetically modified micro-organ expresses said therapeutic polypeptide for a sustained period of at least three months.

In one embodiment, a method of this invention provides increased serum IFN levels in a human subject over a continuous, sustained period of time comprising the steps of: providing an at least one GMMO providing a sustained delivery of a human IFN the micro-organ comprising a vector such as a HDAd or AAV vector, comprising a nucleic acid sequence encoding IFN operably linked to an upstream MAR regulatory sequence, and wherein the nucleic acid further comprises at least one or more additional regulatory sequences; determining IFN secretion levels of the at least one genetically modified micro-organ in vitro; implanting the at least one genetically modified micro-organ in the subject at an effective dosage; and measuring IFN levels in the blood serum of the subject, wherein implantation of the at least one GMMO increases the in vivo serum IFN levels over basal levels for at least three months.

IFNs are multi-functional cytokines that are capable of producing pleitrophic effects on cells, such as anti-viral, anti-proliferative and anti-inflammatory effects. Because of these cellular responses to IFNs, IFN-alpha and IFN-beta have been found to be clinically useful in the treatment of viral, proliferative and inflammatory diseases such as multiple sclerosis, hepatitis B, hepatitis C and several forms of cancer. IFN therapies may also have potential use for the treatment of other inflammatory diseases, viral diseases and proliferative diseases. Thus, a subject in need of IFNs may have one or a combination of the above-mentioned diseases or conditions.

Each of the four major classes of IFNs: alpha (α), beta (β), lambda (λ) and gamma (γ) are encompassed in this invention.

In one embodiment, the therapeutic polypeptide is IFN alpha, in another embodiment, IFN beta, in another embodiment, IFN gamma, and in another embodiment, IFN lambda. In another embodiment, the therapeutic polypeptide is any subtype of IFN alpha, including but not limited to: 1, 2a, 2b, 4, 5, 6, 7, 8, 10, 13, 14, 16, 17, or 21. In another embodiment, the therapeutic polypeptide is IFN omega, epsilon, kappa, or a homolog thereof. In another embodiment, the therapeutic polypeptide is IFN lambda or a homolog thereof. In another embodiment, the therapeutic polypeptide is any subtype of IFN lambda including but not limited to: Interleukin (IL) 28A, IL28B, or IL29. In another embodiment, the therapeutic polypeptide is IFN zeta, nu, tau, delta, or a homolog thereof. In another embodiment, the therapeutic polypeptide is any IFN known in the art.

In one embodiment, the IFN of the GMMO and methods of the instant invention are IFN alpha. In another embodiment, the IFN of the GMMO and methods of the instant invention are IFN alpha2b. In one embodiment, IFN-alpha-2b is a recombinant, non-glycosylated 165-amino acid alpha IFN protein comprising the gene for IFN-alpha-2b from human leukocytes. IFN-alpha-2b is a type I, water-soluble IFN with a molecular weight of 19,271 daltons (19.271 kDa). In one embodiment, IFN-alpha-2b has a specific activity of about 2.6×108 (260 million) International Units/mg as measured by HPLC assay.

In one embodiment, a HDAd vector comprises SEQ ID NO: 22. In another embodiment, a HDAd vector comprises SEQ ID NO: 24. In one embodiment, a HDAd vector comprises a nucleic acid sequence homologous with SEQ ID NO: 22. In another embodiment, a HDAd vector comprises a nucleic acid sequence homologous with SEQ ID NO: 24. In one embodiment, a HDAd vector comprises a nucleic acid sequence homologous with SEQ ID NO: 28. In another embodiment, a HDAd vector comprises a nucleic acid sequence homologous with SEQ ID NO: 28.

In one embodiment, a therapeutic IFN-alpha GMMO of the present invention may be used for the prevention or treatment of hairy cell leukemia, venereal warts, Kaposi's Sarcoma, chronic non-A, non-B hepatitis, hepatitis B, hepatitis C, hepatitis D or a combination thereof. In another embodiment, a therapeutic IFN-alpha GMMO of the present invention may be administered to a subject susceptible to one of the above-mentioned diseases or conditions or has been or will be exposed to an infectious agent, as described herein. In another embodiment, a therapeutic IFN-alpha GMMO may be used for the prevention or treatment of hepatitis C. According to this aspect and in one embodiment, the formulations of the present invention may be administered concurrently or alternately with other hepatitis C treatments, including inter alia, ribavarin, teleprevir, boceprevir or pegylated IFNs, or a combination thereof. In another embodiment, a therapeutic IFN-alpha GMMO may be used for the prevention or treatment of hepatitis D.

In one embodiment, use of a GMMO of the present invention may alleviate symptoms of hepatitis. In one embodiment, use of a GMMO may alleviate symptoms of and/or treat, hepatitis C. In another embodiment, a use of a GMMO may alleviate symptoms of and/or treat, hepatitis B. In yet another embodiment use of a GMMO may alleviate symptoms of and/or treat, hepatitis D. In one embodiment, the disease or disorder is hepatitis C, genotype 1. In another embodiment, the disease or disorder is hepatitis C, genotype 2. In yet another embodiment, the disease or disorder is hepatitis C, genotype 3. In still another embodiment, the subject has hepatitis C, genotype 4, 5, 6, 7, 8, 9, 10 or 11. In a further embodiment, the subject has hepatitis C, with a combination of genotypes.

In some embodiments, methods of this invention treat hepatitis in a subject, wherein the hepatitis is hepatitis D. In one embodiment, the hepatitis D is chronic hepatitis D. In another embodiment, the hepatitis viral infection is caused by a combination of hepatitis B and hepatitis D infection.

As used herein, “hepatitis B” refers to an irritation and swelling (inflammation) of the liver due to infection with the hepatitis B virus (HBV). Hepatitis B infection can be spread through having contact with the blood, semen, vaginal fluids, and other body fluids of someone who already has a hepatitis B infection. Infection can be spread through: blood transfusions (not common in the United States); direct contact with blood in health care settings; sexual contact with an infected person; tattoo or acupuncture with unclean needles or instruments; shared needles during drug use; shared personal items (such as toothbrushes, razors, and nail clippers) with an infected person; and the hepatitis B virus can be passed to an infant during childbirth if the mother is infected.

As used herein, “hepatitis C” refers to an infectious disease affecting primarily the liver, caused by the hepatitis C virus (HCV). The infection is often asymptomatic, but chronic infection can lead to scarring of the liver and ultimately to cirrhosis, which is generally apparent after many years. In some cases, those with cirrhosis will go on to develop liver failure, liver cancer or life-threatening esophageal and gastric varices. HCV is spread primarily by blood-to-blood contact associated with intravenous drug use, poorly sterilized medical equipment and transfusions.

As used herein, “hepatitis D” refers to an inflammation of the liver caused by the hepatitis D virus (HDV). HDV is a defective RNA virus that cannot replicate autonomously but can assemble as a virion only if provided with a lipoprotein envelop by the hepatitis B virus (HBV). Therefore, transmission of HDV can occur only via simultaneous infection with HBV (co-infection) or via superimposition on chronic hepatitis B or hepatitis B carrier state (super-infection). Whereas co-infection generally leads to a self-limiting acute hepatitis, super-infection causes a severe acute hepatitis that in 80-90% of infected people progresses to chronicity (chronic hepatitis D).

In another embodiment, a GMMO may be used or evaluated alone or in conjunction with chemotherapeutic agents in a variety of other cellular proliferation disorders, including chronic myelogenous leukemia, multiple myeloma, superficial bladder cancer, skin cancers (including, inter alia, basal cell carcinoma and malignant melanoma), renal cell carcinoma, ovarian cancer, low grade lymphocytic and cutaneous T cell lymphoma, and glioma. In another embodiment, a long-lasting IFN-alpha formulation may be used for the prevention or treatment of solid tumors that arise from lung, colorectal and breast cancer, alone or with other chemotherapeutic agents. In another embodiment, a GMMO providing sustained delivery of IFN, may be used for the treatment of multiple sclerosis. In another embodiment, a long-lasting IFN-alpha formulation may be used for the prevention or treatment of histiocytic diseases, which in one embodiment is Erdheim-Chester disease (ECD), which in one embodiment is a potentially fatal disorder that attacks the body's connective tissue and in one embodiment is caused by the overproduction of histiocytes, which in one embodiment, accumulate in loose connective tissue, causing it to become thickened and dense. In another embodiment, a GMMO providing sustained delivery of IFN may be used for the prevention or treatment of severe ocular Behcet's disease.

In one embodiment, the IFN alpha gene has a nucleic acid sequence that corresponds to that set forth in Genbank Accession Nos: K01900; M11003; or M71246, or encodes a protein sequence that corresponds to that set forth in Genbank Accession Nos: AAA52716; AAA52724; or AAA52713. In one embodiment, the IFN beta gene has a nucleic acid sequence that corresponds to that set forth in Genbank Accession Nos: M25460; AL390882; or CH236948, or encodes a protein sequence that corresponds to that set forth in Genbank Accession Nos: AAC41702; CAH70160; or EAL24265. In one embodiment, the IFN gamma gene has a nucleic acid sequence that corresponds to that set forth in Genbank Accession Nos: J00219; AF506749; NM_{—}000619; or X62468, or encodes a protein sequence that corresponds to that set forth in Genbank Accession Nos: AAB59534; AAM28885; NP_{—}000610; or CAA44325. In another embodiment, the IFN alpha gene has a nucleic acid sequence as presented in SEQ ID NO: 19, while in another embodiment, the IFN alpha gene has a nucleic acid sequence as presented in SEQ ID NO: 20, while in another embodiment, the IFN alpha gene has an amino acid sequence as presented in SEQ ID NO: 21. In another embodiment, the IFN alpha gene has a nucleic acid that is homologous to that presented in SEQ ID NO: 19, while in another embodiment, the IFN alpha gene has a nucleic acid that is homologous to that presented in SEQ ID NO: 20, while in another embodiment, the IFN alpha gene has an amino acid sequence that is homologous to that presented in SEQ ID NO: 21.

### EPO Expression Cassettes

CAG-wt-hEPO and CAG-opt-hEPO: In certain embodiments, any of the expression cassettes described herein may be transduced into the micro-organ to generate the genetically modified micro-organs of the invention. For example, the CAG-wtEPO expression cassette encodes a wild-type (wt) erythropoietin polypeptide (EPO) (SEQ ID NO: 3), and CAG-opt-hEPO expression cassette encodes an optimized EPO (SEQ ID NO:2). The nucleic acid sequence of the CAG-wtEPO expression cassette is presented as SEQ ID NO: 17. The nucleic acid sequence of the CAG-opt-hEPO expression cassette is presented as SEQ ID NO: 15. The CAG-wtEPO and CAG-opt-hEPO includes a CAG promoter sequence (SEQ ID NO: 7); human EPO intron-less gene from ATG to the stop codon (SEQ ID NO: 1 and SEQ ID NO:2, respectively); and SV40 poly A sequence (SEQ ID NO: 9). The GMMOs of the invention may comprise the CAG-wtEPO expression cassette (SEQ ID NO: 17), or a sequence that is 80%, 85%, 90%, or 95% identical to SEQ ID NO:17. The GMMOs of the invention may comprise the CAG-opt-hEPO expression cassette (SEQ ID NO: 15), or a sequence that is 80%, 85%, 90%, or 95% identical to SEQ ID NO:15. The GMMOs of the invention may further comprise an HDAd or AAV vector comprising SEQ ID NO: 17 or SEQ ID NO: 15, or a sequence that is 80%, 85%, 90%, or 95% identical to SEQ ID NO: 17 or SEQ ID NO: 15. In one embodiment the GMMOs comprise an HDAd vector comprising CAG-wt-hEPO, wherein the GMMO comprises the nucleic acid sequence of SEQ ID NO: 14, or a sequence that is 80%, 85%, 90%, or 95% identical to SEQ ID NO: 14. In one embodiment the GMMOs comprise an AAV vector comprising CAG-wt-hEPO. In one embodiment the GMMOs comprise an HDAd vector comprising CAG-opt-hEPO, wherein the GMMO comprises the nucleic acid sequence of SEQ ID NO: 16, or a sequence that is 80%, 85%, 90%, or 95% identical to SEQ ID NO: 16. In one embodiment the GMMOs comprise an AAV vector comprising CAG-opt-hEPO.

MAR-opt-hEPO-WPRE (also referred to herein as MAR-CAG-opt-hEPO-WPRE): In certain embodiments, any of the expression cassettes described herein may be transduced into the micro-organ to generate the genetically modified micro-organs of the invention. For example, the MAR-opt-hEPO-WPRE expression cassette (also referred to herein as MAR-CAG-opt-hEPO-WPRE) encodes an optimized erythropoietin polypeptide (EPO) (SEQ ID NO: 2). The nucleic acid sequence of the MAR-opt-hEPO-WPRE expression cassette is presented as SEQ ID NO: 13. The MAR-opt-hEPO-WPRE includes a human IFNβ S/MAR regulatory sequence (SEQ ID NO: 5); a CAG promoter (SEQ ID NO: 7); optimized human EPO intron-less gene from ATG to the stop codon (SEQ ID NO: 2) WPRE regulatory sequence (SEQ ID NO: 8); and SV40 poly A sequence (SEQ ID NO: 9). The optimized human EPO gene was optimized for human codon usage. The GMMOs of the invention may comprise the MAR-opt-hEPO-WPRE expression cassette (SEQ ID NO: 13), or a sequence that is 80%, 85%, 90%, or 95% identical to SEQ ID NO: 13. The GMMOs of the invention may further comprise an HDAd or AAV vector comprising SEQ ID NO: 13 or a sequence that is 80%, 85%, 90%, or 95% identical to SEQ ID NO: 13. In one embodiment the GMMOs comprise an HDAd vector comprising MAR-CAG-opt-hEPO-WPRE, wherein the GMMO comprises the nucleic acid sequence of SEQ ID NO: 12, or a sequence that is 80%, 85%, 90%, or 95% identical to SEQ ID NO: 12. In one embodiment the GMMOs comprise an AAV vector comprising MAR-CAG-opt-hEPO-WPRE, wherein the GMMO comprises the nucleic acid sequence of SEQ ID NO: 26, or a sequence that is 80%, 85%, 90%, or 95% identical to SEQ ID NO: 26.

MAR-EF1α-opt-hEPO: The MAR-EF1α-opt-hEPO expression cassette present in the GMMOs of the invention is schematically illustrated in FIG. 3. The MAR-EF1α-opt-hEPO expression cassette encodes an optimized erythropoietin polypeptide (EPO) (SEQ ID NO: 2). The nucleic acid sequence of the MAR-EF1α-opt-hEPO expression cassette is presented as SEQ ID NO: 11. The MAR-EF1α-opt-hEPO includes a CpG free human β-globin MAR regulatory sequence (SEQ ID NO: 6); an EF1α promoter (SEQ ID NO: 18); optimized human EPO intron-less gene from ATG to the stop codon (SEQ ID NO: 2); SV40 poly A sequence (SEQ ID NO: 9); and human IFNβ S/MAR regulatory sequence (SEQ ID NO: 5). The optimized human EPO gene was optimized for human codon usage. Also encompassed in the invention is an expression cassette comprising SEQ ID NO: 11 except that the non-optimized version of hEPO (SEQ ID NO: 1) is used in place of the optimized version. The GMMOs of the invention may comprise the MAR-EF1α-opt-hEPO expression cassette (SEQ ID NO: 11), or a sequence that is 80%, 85%, 90%, or 95% identical to SEQ ID NO: 11. The GMMOs of the invention may further comprise an HDAd or AAV vector comprising SEQ ID NO: 11 or a sequence that is 80%, 85%, 90%, or 95% identical to SEQ ID NO: 11. The HDAd and AAV vector backbones may be CpG free. In one embodiment the GMMOs comprise an HDAd vector comprising MAR-EF1α-opt-hEPO, wherein the GMMO comprises the nucleic acid sequence of SEQ ID NO: 10, or a sequence that is 80%, 85%, 90%, or 95% identical to SEQ ID NO: 10. In one embodiment the GMMOs comprise an AAV vector comprising MAR-EF1α-opt-hEPO, wherein the GMMO comprises the nucleic acid sequence of SEQ ID NO: 27, or a sequence that is 80%, 85%, 90%, or 95% identical to SEQ ID NO: 27.

In one embodiment, the terms “identity,” “homology”, “homologue” or “homologous”, in any instance, indicate that the sequence referred to, exhibits, in one embodiment at least 70% correspondence with the indicated sequence. In another embodiment, the nucleic acid sequence exhibits at least 72% correspondence with the indicated sequence. In another embodiment, the nucleic acid sequence exhibits at least 75% correspondence with the indicated sequence. In another embodiment, the nucleic acid sequence exhibits at least 77% correspondence with the indicated sequence. In another embodiment, the nucleic acid sequence exhibits at least 80% correspondence with the indicated sequence. In another embodiment, the nucleic acid sequence exhibits at least 82% correspondence with the indicated sequence. In another embodiment, the nucleic acid sequence exhibits at least 85% correspondence with the indicated sequence. In another embodiment, the nucleic acid sequence exhibits at least 87% correspondence with the indicated sequence. In another embodiment, the nucleic acid sequence exhibits at least 90% correspondence with the indicated sequence. In another embodiment, the nucleic acid sequence exhibits at least 92% correspondence with the indicated sequence. In another embodiment, the nucleic acid sequence exhibits at least 95% or more correspondence with the indicated sequence. In another embodiment, the nucleic acid sequence exhibits 95%-100% correspondence to the indicated sequence. Similarly, reference to a correspondence to a particular sequence includes both direct correspondence, as well as homology to that sequence as herein defined.

Homology may be determined by computer algorithm for sequence alignment, methods of which are well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of nucleic acid sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y.). In one embodiment, methods of hybridization may be carried out under moderate to stringent conditions. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In one embodiment, the present invention provides therapeutic formulations comprising micro-organs and methods of use thereof. In one embodiment, the preparation of therapeutic micro-organs comprises (a) obtaining a plurality of micro-organ explants from a donor subject, each of the plurality of micro-organ explants comprises a population of cells, each of the plurality of micro-organ explants maintaining a microarchitecture of an organ from which it is derived and at the same time having dimensions selected so as to allow diffusion of adequate nutrients and gases to cells in the micro-organ explants and diffusion of cellular waste out of the micro-organ explants so as to minimize cellular toxicity and concomitant death due to insufficient nutrition and accumulation of waste in the micro-organ explants; and (b) genetically modifying the plurality of micro-organ explants, so as to obtain a plurality of GMMO explants.

Methods for the preparation and processing of micro-organs into GMMOs are disclosed in WO2004/099363 and WO2011/159758. Micro-organs comprise tissue dimensions defined such that diffusion of nutrients and gases into every cell in the three-dimensional micro-organ, and sufficient diffusion of cellular wastes out of the explant, is assured. Ex vivo maintenance of the micro-organs, which in one embodiment, is in minimal media, can continue for an extended period of time, whereupon controlled ex vivo transduction incorporating desired gene candidates within cells of the micro-organs using viral or non-viral vectors occurs, thus creating GMMOs.

Typically the nucleic acid sequence is subcloned within a particular vector, depending upon the preferred method of introduction of the sequence to within the micro-organs, as described hereinabove. Once the desired nucleic acid segment is subcloned into a particular vector it thereby becomes a recombinant vector.

In one embodiment, micro-organs are incubated at 32° C. before and after genetic modification, while in another embodiment, they are incubated at 37° C. In another embodiment, micro-organs are incubated at 33° C., 34° C., 35° C., 36° C., 38° C., 39° C., 40° C., 28° C., 30° C., 31° C., or 25° C.

In one embodiment, micro-organs are incubated at 10% CO₂ before and after genetic modification, while in another embodiment, they are incubated at 5% CO₂. In another embodiment, micro-organs are incubated at 12% CO₂, 15% CO₂, 17% CO₂, or 20% CO₂. In another embodiment, micro-organs are incubated at 0% CO₂, 2% CO₂, 6% CO₂, 7% CO₂, 8% CO₂, or 9% CO₂.

In another embodiment, incubation temperatures, CO₂ concentrations, or a combination thereof may be kept at a single temperature or concentration before, during, and after genetic modification, while in another embodiment, incubation temperatures, CO₂ concentrations, or a combination thereof may be adjusted at different points before, during, and after genetic modification of micro-organs.

In another embodiment, micro-organs are incubated at 85-100% humidity, which in one embodiment is 95% humidity, in another embodiment, 90% humidity, and in another embodiment, 98% humidity.

In one embodiment, the levels of therapeutic nucleic acids or polypeptides may be detected using any method known in the art. The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. In one embodiment, ELISA, Western blots, or radioimmunoassay may be used to detect proteins.

Thus, in one embodiment, therapeutic polypeptide or nucleic acid expression levels are measured in vitro, while in another embodiment, therapeutic polypeptide or nucleic acid expression levels are measured in vivo. In one embodiment, in vitro determination of polypeptide or nucleic acid expression levels, which in one embodiment, is EPO levels and in another embodiment, IFN-alpha levels, allows for determination of the number of micro organs to be implanted in a patient via determining the secretion level of a therapeutic agent by each micro-organ in vitro; knowing the target dose of therapeutic agent required by the patient, and calculating the number of GMMO that will provide that target dose after implantation into the patient.

In another preferred embodiment of this invention, polynucleotide(s) can also include trans-, or cis-acting enhancer or suppresser elements that regulate either the transcription or translation of endogenous genes expressed within the cells of the micro-organs, or additional recombinant genes introduced into the micro-organs. Numerous examples of suitable translational or transcriptional regulatory elements, which can be utilized in mammalian cells, are known in the art.

For example, transcriptional regulatory elements comprise cis- or trans-acting elements, which are necessary for activation of transcription from specific promoters [(Carey et al., (1989), J. Mol. Biol. 209:423-432; Cress et al., (1991), Science 251:87-90; and Sadowski et al., (1988), Nature 335:5631-564)].

Translational activators are exemplified by the cauliflower mosaic virus translational activator (TAV) [see for example, Futterer and Hohn, (1991), EMBO J. 10:3887-3896]. In this system a bi-cistronic mRNA is produced. That is, two coding regions are transcribed in the same mRNA from the same promoter. In the absence of TAV, only the first cistron is translated by the ribosomes, however, in cells expressing TAV, both cistrons are translated.

The polynucleotide sequence of cis-acting regulatory elements can be introduced into cells of micro-organs via commonly practiced gene knock-in techniques. For a review of gene knock-in/out methodology see, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO 93/14200, WO 94/06908 and WO 94/28123 also provide information.

Down-regulation of endogenous sequences may also be desired, in order to assess production of the recombinant product exclusively. Toward this end, antisense RNA may be employed as a means of endogenous sequence inactivation. Exogenous polynucleotide(s) encoding sequences complementary to the endogenous mRNA sequences are transcribed within the cells of the micro-organ. Down regulation can also be effected via gene knock-out techniques, practices well known in the art (“Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988).

Over expression of the recombinant product may be desired as well. Over expression may be accomplished by providing a high copy number of one or more coding sequences in the respective vectors. These exogenous polynucleotide sequences can be placed under transcriptional control of a suitable promoter of a mammalian expression vectors to regulate their expression. In another embodiment, multiple copies of the same gene or of several related genes may be used as a means to increase polypeptide or nucleic acid expression. In one embodiment, expression is stabilized by DNA elements, which in one embodiment are scaffold/matrix-associating regions (S/MARs) or scaffold-associating regions (SARs).

In one embodiment, decreasing the in-vivo GMMO processing time significantly improves the in-vivo performance of AAV and HDAd transduced GMMOs. Therefore, in one aspect of the invention, methods are provided for processing GMMOs wherein the in-vitro processing time is reduced from what is standard in practice. The standard in-vitro processing time for GMMOs is 9, 10, 11, 12, 13, 14, or 15 days. Thus, in one embodiment of the present invention, the GMMO is processed in-vitro for less than 9 days. In another embodiment, the GMMO is processed in-vitro for less than 8, 7, 6, 5, 4, 3, 2, or 1 day. In some embodiments, the GMMO is processed in-vitro for 8, 7, 6, 5, 4, 3, 2, or 1 day, or for a range of time between any two of the above numbers of days. The term “in-vitro processing time” is meant to include the time after removal of the MO from the patient until re-implantation into the subject.

In one embodiment, micro-organs are maintained ex vivo for a period of time, which may range from several hours to several months. In one embodiment, maintenance ex vivo refers to maintenance of a micro-organ following ex vivo genetic manipulation using a viral vector, i.e., maintenance of a GMMO. In another embodiment, maintenance ex vivo refers to maintenance of a micro-organ prior to genetic manipulation thereof.

In one embodiment, GMMOs are maintained for several days, and in another embodiment, for several weeks prior to implantation. In one embodiment, micro-organs are maintained for between 3-7 days prior to implantation. In one embodiment, micro-organs are maintained for 2 days prior to implantation. In one embodiment, micro-organs are maintained for 3 days prior to implantation. In another embodiment, micro-organs are maintained for 4 days prior to implantation. In another embodiment, micro-organs are maintained for 5 days prior to implantation. In yet another embodiment, micro-organs are maintained for 6 days prior to implantation. In still another embodiment, micro-organs are maintained for 7 days prior to implantation. In a further embodiment, micro-organs are maintained for 8 days prior to implantation. In one embodiment, micro-organs are maintained for between 1-9 days prior to implantation. In one embodiment, micro-organs are maintained for between 9-14 days prior to implantation. In one embodiment, micro-organs are maintained for between two to four weeks prior to implantation. In one embodiment, micro-organs are maintained for three weeks prior to implantation. In one embodiment, micro-organs are maintained for four weeks or more prior to implantation. In yet another embodiment, micro-organs are maintained for at least 9 days prior to implantation. In one embodiment, micro-organs are maintained for at least 3 days prior to implantation. In another embodiment, micro-organs are maintained for at least 5 days prior to implantation. In yet another embodiment, micro-organs are maintained for at least 7 days prior to implantation.

Without being limited by theory, in one embodiment, said incubation allows cells to process and break down viral proteins, which in one embodiment are viral capsids, present as a result of viral vector transduction. In one embodiment, such a turnover of capsid proteins occurs within 2-3 days, so that, in one embodiment, little if any viral capsid proteins remain by the 10th day ex vivo. In one embodiment, the breaking down of viral capsids further reduces the immunogenicity of the formulations of the instant invention and may increase the expression levels and expression duration of the gene or genes of interest in vivo. In another embodiment, said incubation allows the early vector-induced innate immune responses to occur in vitro, which in one embodiment, will not persist beyond 24 hours in the absence of vector gene transcription. In another embodiment, the later adaptive responses that normally follow the administration of transcription-competent first-generation vectors, which are predominantly characterized in one embodiment, by lymphocyte infiltration and in another embodiment by induction of vector-specific CTL's, are not elicited by the vectors.

In one embodiment, the ex vivo micro-organ is exposed to vector at a dosage of 1×10⁷-1×10¹² infectious particles(ip)/GMMO, in another embodiment at a dosage of −1×10⁸-1×10¹¹ ip/GMMO, while in a further embodiment, 1×10⁹-5×10¹⁰ ip/GMMO, and in still another embodiment, 1×10¹⁰-5×10¹⁰ ip/GMMO. In one embodiment, a dosage of 1.5×10¹⁰ ip/GMMO is used. In one embodiment, the ex vivo micro-organ is exposed to a dosage of not less than 1.5×10⁶ viral particles/GMMO and not more than 1×10¹² viral particles/GMMO

In one embodiment, growth factors are used to increase the number of cells in the micro-organs.

In one embodiment, in vitro expression can be assessed prior to implantation, enabling the possibility for in vitro to in vivo correlation studies of expressed recombinant proteins.

In some embodiments of the invention, the amounts of tissue sample including genetically modified cell(s) to be implanted are determined from one or more of: corresponding amounts of the therapeutic agent of interest routinely administered to such subjects based on regulatory guidelines, specific clinical protocols or population statistics for similar subjects, corresponding amounts of the therapeutic agent such as protein of interest specifically to that same subject in the case that he/she has received it via injections or other routes previously, subject data such as weight, age, physical condition, clinical status, pharmacokinetic data from previous tissue sample which includes a genetically modified cell administration to other similar subjects, response to previous tissue sample which includes genetically modified cell administration to that subject, or a combination thereof. Thus, in one embodiment, the in vitro level of expression of gene products by one or more micro-organs is determined, a relationship between in vitro and in vivo therapeutic polypeptide or nucleic acid expression levels is determined or estimated, and the number of micro-organs to be implanted in a particular patient is determined based on the calculated or estimated relationship. The dosage of the therapeutic agent may be adjusted as described previously (WO2004/099363).

In one embodiment, a micro-organ or a GMMO may be maintained in vitro for a proscribed period of time until they are needed for implantation into a host. In one embodiment, a micro-organ or a GMMO may be maintained or stored in culture for between 1-3 days, 2-4 days, 2-5 days, 3-6 days, 1-7 days, between 1-8 weeks, or for 1-4 months. In one embodiment, a micro-organ or a GMMO may be maintained or stored in culture for at least 3 days. In another embodiment for at least 4 days, alternatively for at least 5 days, in yet another embodiment for at least 6 days, in still another embodiment, for at least 7 days, in a further embodiment for at least 8 days, and in another embodiment, for at least 9 days. In another embodiment, the therapeutic agent, left in the supernatant medium surrounding the tissue sample, can be isolated and injected or applied to the same or a different subject.

Alternatively or additionally, a GMMO can be cryogenically preserved by methods known in the art, for example, without limitation, gradual freezing (0° C., −20° C., −80° C., −196° C.) in DMEM containing 10% DMSO, immediately after being formed from the tissue sample or after genetic alteration.

Administration of the GMMO of the invention may be by implanting into the subject in need. According to this aspect and in one embodiment, formulations of the instant invention may be implanted subcutaneously. In another embodiment, formulations may be implanted intradermally. In yet another embodiment, formulations may be implanted subdermally.

In one embodiment, the GMMO of the invention may be implanted a single time for acute treatment of temporary conditions, or may be implanted more than one time, especially in the case of progressive, recurrent, or degenerative disease. In one embodiment, one or more GMMO of the invention may be administered simultaneously, or in another embodiment, they may be administered in a staggered fashion. In one embodiment, the staggered fashion may be dictated by the stage or phase of the disease. In certain embodiments, methods of this invention further comprise a step of implanting at a later date to a subject, at least one additional GMMO that expresses and secretes a therapeutic polypeptide, the micro-organ comprising a vector comprising a nucleic acid sequence encoding the therapeutic polypeptide operably linked to an upstream MAR regulatory sequence, and wherein the nucleic acid further comprises at least one additional regulatory sequence. In one embodiment, the therapeutic polypeptide is human erythropoietin. In another embodiment, the therapeutic polypeptide is human interferon.

In one embodiment, the micro-organ is implanted at a desired location in the subject in such a way that at least a portion of the cells of the micro-organ remain viable. In one embodiment of this invention, at least about 5%, in another embodiment of this invention, at least about 10%, in another embodiment of this invention, at least about 20%, in another embodiment of this invention, at least about 30%, in another embodiment of this invention, at least about 40%, and in another embodiment of this invention, at least about 50% or more of the cells remain viable after administration to a subject. In a further embodiment, at least about 60% of the cells of the micro-organ remain viable, while in another embodiment, at least about 70%, and in yet another embodiment, at least about 80%, while in still another embodiment, at least about 90% or more of the cells remain viable after administration to a subject. The period of viability of the cells after administration to a subject can be as short as a few hours, e.g., twenty-four hours, to a few days, to as long as a few weeks to months or years.

Micro-organ implantation within a recipient subject provides for a sustained dosage of the recombinant product, for example a therapeutic polypeptide. The micro-organs may be prepared, prior to implantation, for efficient incorporation within the host facilitating, for example, formation of blood vessels within the implanted tissue. Recombinant products may therefore be delivered immediately to peripheral recipient circulation, following production. Alternatively, micro-organs may be prepared, prior to implantation, to prevent cell adherence and efficient incorporation within the host.

In one embodiment, a GMMO is encapsulated. In another embodiment, a GMMO is not encapsulated.

Implantation of GMMOs according to the present invention can be effected via standard surgical techniques or via injecting micro-organ preparations into the intended tissue regions of the mammal utilizing specially adapted syringes employing a needle of a gauge suitable for the administration of micro-organs. In another embodiment, a catheter is employed for implanted micro-organs. In one embodiment, any of the implantation methods described in WO2004/099363 or WO2013/118109 may be used in this invention.

In one embodiment, micro-organs are implanted subcutaneously, intradermally, subdermally, intramuscularly, intraperitoneally or intragastrically. In one embodiment, the term implanted excludes being grafted as a split-thickness or full-thickness skin graft. In one embodiment of the present invention, the donor micro-organs utilized for implantation are preferably prepared from an organ tissue of the recipient mammal (i.e. autologous), or a syngeneic mammal, although allogeneic and xenogeneic tissue can also be utilized for the preparation of the micro-organs providing measures are taken prior to, or during implantation, so as to avoid graft rejection. In another embodiment, the micro-organ is not inserted into or encapsulated by biocompatible immuno-protected material such as rechargeable, non-biodegradable or biodegradable devices.

In one embodiment, viral turnover or elimination from cells ex vivo is enhanced via techniques know in the art, such as physical methods, which in one embodiment is heating, use of antiviral agents, agents which stimulate viral turnovers by cells, etc.

In one embodiment, while the GMMO of the present invention increase the level and duration of polypeptide expression, the levels of polypeptide expression do not remain elevated indefinitely.

In contrast to other methods involving transient transduction of cells, or cells that turn over rapidly, the long-lasting therapeutic formulation of the instant invention comprises cells that are not quickly replicating. Therefore, the GMMO produces a stable protein from a stable construct and is expected to continue producing the protein already characterized.

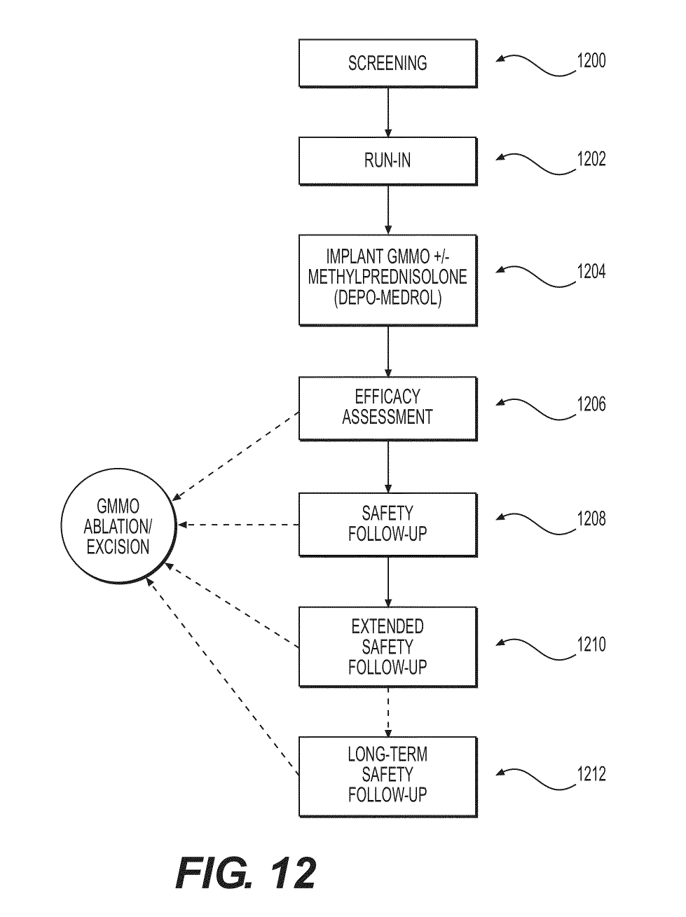

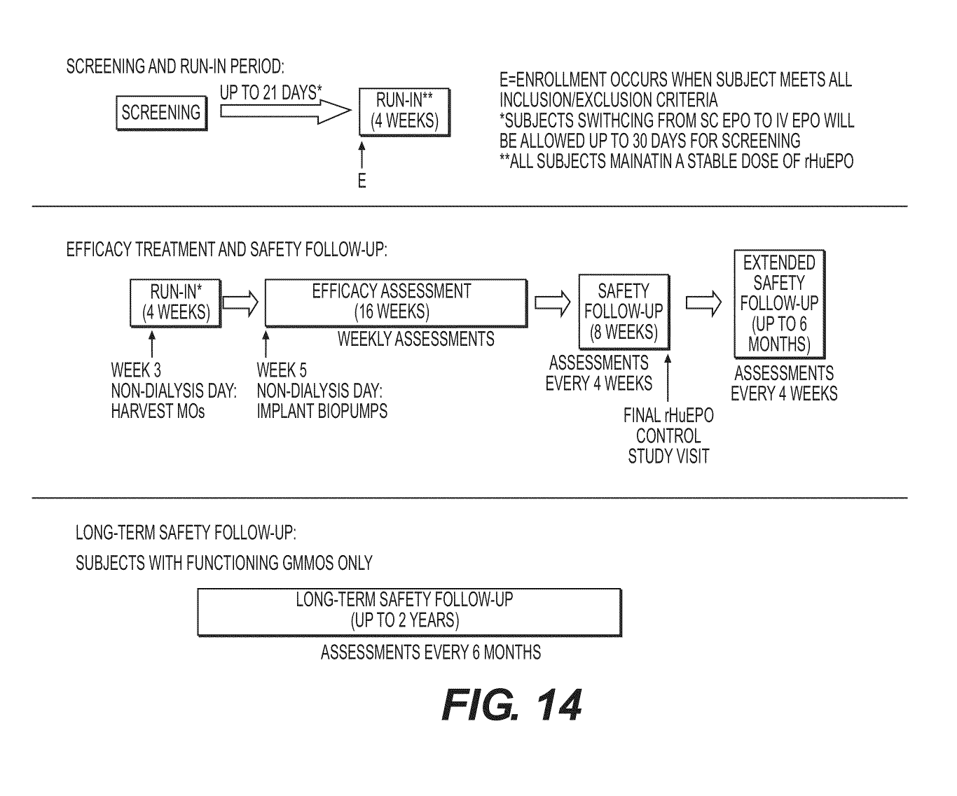

Reference is now made to FIG. 12, which schematically illustrates a flowchart of the steps for evaluating the safety and effectiveness of administering therapeutic GMMOs, in the case of the figure a hEPO GMMO transduced with HDAd MAR-EF1α-opt-hEPO, for treating anemia in hemodialysis patients according to some exemplary embodiments of the invention. Although the discussion below refers to an EPO GMMO, the methods and steps thereof described in FIG. 12 are applicable to any therapeutic GMMO of the invention. Further, FIG. 14 shows an overview of the steps presented in FIG. 12 in reference to treating a human subject with an EPO GMMO and the relationship including duration of each step. In one embodiment, the steps described in FIG. 12 are embodiments of a clinical trial. In one embodiment, the subjects are suffering from end-stage renal disease (ESRD). In another embodiment, subjects are suffering from anemia secondary to ESRD. In one embodiment, subjects are receiving hemodialysis. In one embodiment, subjects receive hemodialysis at least twice per week. In another embodiment, subjects receive hemodialysis at least three times per week. In a certain embodiment, subjects include a human in need of increased, or maintained, or increased and maintained Hb levels. In yet another embodiment, steps presented in FIG. 12 may be added-to, removed and/or revised.

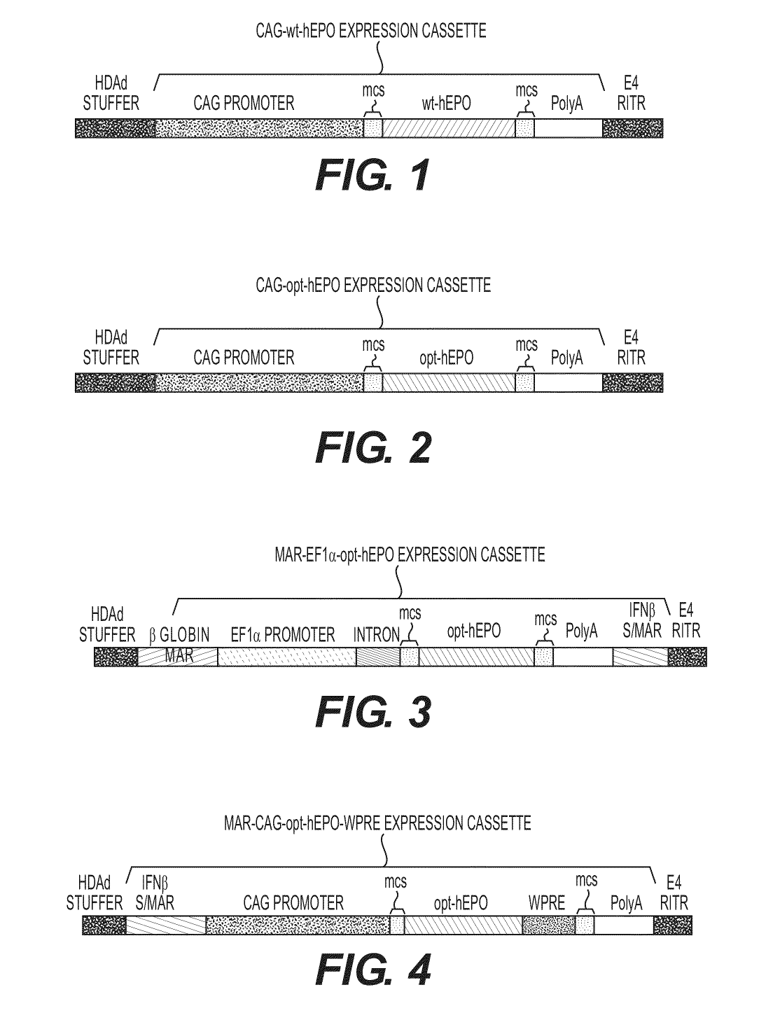

For some embodiments of this invention, a method of transducing a DMO to create an EPO GMMO is described below in the Examples. In one embodiment of this invention, DMOs may be transduced with an hEPO producing vector of this invention. In one embodiment, the vector comprises HDAd-MAR-EF1α-opt-hEPO (FIG. 3; SEQ ID NO: 10). In still another embodiment, the vector comprises HDAd-MAR-CAG-opt-hEPO-WPRE (FIG. 4; SEQ ID NO: 12). In a further embodiment, the vector comprises any HDAd viral vector or any AAV viral vector expressing and secreting human EPO and a MAR element. In other embodiments, any nucleic acid encoding a therapeutic polypeptide, may be substituted for the wt-hEPO or opt-hEPO expression cassettes illustrated in FIGS. 1-4. For instance, a MAR-EF1alpha-optIFNalpha expression cassette (SEQ ID NO: 23) would comprise optimized IFNa nucleic acid in place of opt-EPO in the cassette schematically represented by FIG. 3. Alternatively, a MAR-CAG-optIFNα-WPRE expression cassette (SEQ ID NO: 25) would comprise optimized IFNa nucleic acid in place of optEPO in the cassette schematically represented by FIG. 4.

In one embodiment, immediately following EPO GMMO implantation, methylprednisolone is administered subcutaneously around each GMMO implantation site. In another embodiment, methylprednisolone is administered subcutaneously around a GMMO implantation site concurrent with GMMO implantation. In one embodiment, methylprednisolone is not administered as part of EPO GMMO treatment.

In one embodiment, an anti-inflammatory, anti-proliferation or anti-oxidant, or any combination thereof is administered as part of EPO GMMO treatment. In one embodiment, an anti-inflammatory, anti-proliferation or anti-oxidant used in methods of this invention may include Vitamin C, N-Acetyl Cysteine, Caspase-1 Inhibitor (Z-Wehd-Fmk), Cytosine, Pirfendone, Tempol, Cathepsin B inhibitor (CA-074-OME), Demecolcine, zVAD (pan caspase inhibitor), Minocycline hydrochloride (caspase 1 and 3 inhibitor), Actemra (IL-6 inhibitor), Aspirin (cox inhibitor), MIF antagonist (macrophage migration inhibitory factor), Infliximab (Anti TNF), Mitomycin C, Resveratrol, Hyaluronic Acid, triamcinolone acetonide, triamcinolone hexacetonide and methylprednisolone. In one embodiment, an anti-inflammatory, anti-proliferation or anti-oxidant used in methods of this invention is administered subcutaneously around each EPO GMMO site. In one embodiment, an anti-inflammatory, anti-proliferation or anti-oxidant is administered weekly. In another embodiment, an anti-inflammatory, anti-proliferation or anti-oxidant is administered bi-weekly. In yet another embodiment, an anti-inflammatory, anti-proliferation or anti-oxidant is administered semi-weekly.

In alternate embodiments, methylprednisolone is administered subcutaneously around each GMMO implantation site following implantation of GMMO(s) expressing and secreting any therapeutic polypeptide. In one embodiment, methylprednisolone is administered weekly following GMMO implantation. In another embodiment, methylprednisolone is administered bi-weekly (every two weeks) following GMMO implantation. In yet another embodiment, methylprednisolone is administered semi-weekly (twice a week) following GMMO implantation. In one embodiment, methylprednisolone is administered a single time at the time of GMMO implantation.

In one embodiment, a dose of an anti-inflammatory, anti-proliferation or anti-oxidant, for example methylprednisolone, is delivered in multiple injections at a GMMO implantation site. In one embodiment, a dose is delivered in at least one injection. In another embodiment, a dose is delivered in at least two injections. In yet another embodiment, a dose is delivered in at least three injections. In still another embodiment, a dose is delivered in at least four injections. In one embodiment, a total dose of methylprednisolone per GMMO is 12 mg. In another embodiment, a total dose of methylprednisolone per GMMO may be between 1-120 mg. For example, a dose of 12 mg delivered in 3 injections would be administered as 4 mg methylprednisolone per injection. In one embodiment, the total dose of methylprednisolone per GMMO implantation site does not exceed 120 mg. In another embodiment, the total dose of methylprednisolone per GMMO implantation site is greater than 120 mg.

The injection site of an anti-inflammatory, anti-proliferation or anti-oxidant, for example methylprednisolone injection site, should be as close as possible to the GMMO. In one embodiment, the injection site is no more than 1 mm from the GMMO. In another embodiment, the injection site is no more than 2 mm from the GMMO. In yet another embodiment, the injection site is no more than 3 mm from the GMMO. In still another embodiment, the injection site is no more than 4 mm from the GMMO. In a further embodiment, the injection site is no more than 5 mm from the GMMO. In another embodiment, the injection site is no more than 6 mm from the GMMO. In yet another embodiment, the injection site is no more than 7 mm from the GMMO. In still another embodiment, the injection site is no more than 8 mm from the GMMO. In a further embodiment, the injection site is no more than 9 mm from the GMMO. In another embodiment, the injection site is no more than 10 mm from the GMMO. FIG. 13 shows one embodiment of this invention having three injections of methylprednisolone per implanted GMMO, each injection not more than 5 mm away from the centerline of the implanted GMMO.

Following GMMO implantation and administration of an anti-inflammatory agent such as methylprednisolone, a topical steroid, for example a corticosteroid such as betamethasone valerate may be applied at the area of implantation. Application of a topical steroid may in one embodiment be weekly, in another embodiment be bi-weekly, in yet another embodiment be daily. In one embodiment, administration of a topical steroid begins one week from implantation of a GMMO. In another embodiment, administration of a topical steroid is according to knowledge in medical practice of the time. The duration of application of topical steroid is in one embodiment for at least two weeks, in still another embodiment for at least three weeks, in a further embodiment for at least four weeks, in another embodiment for at least five weeks, in yet another embodiment for at least six week, in still another embodiment for at least seven weeks, and in a further embodiment for at least eight weeks.

In some embodiments, supplemental rHuEPO injections (Epoetin-alfa-Epogen) may be given at any time during the study to subjects when Hb values fall below the target range. If the Hb level drops below 9 g/dL on 3 consecutive weekly assessments (approximately 2 calendar weeks duration), or falls below 8 g/dL on any single measurement, supplemental rHuEPO may be administered using a standard algorithm that is based on the Hb level and the rate of Hb level rise or fall. When the Hb level returns to the target range on 2 consecutive weekly assessments, the rHuEPO injections may be stopped or reduced in dose.

During the assessment and follow-up phases, steps at blocks 1206-1212, and based on Hb response following EPO GMMO implantation there may be a need to modify the dose by removing or ablating or inactivating one or more additional EPO GMMOs. For example, if the mean Hb level is within the target range of 9-11 g/dL for two consecutive weeks and has changed less than 1.0 g/dL from the mean Hb level during the Run-In Phase, there will be no need to change the EPO GMMOs dose. Alternatively, if the mean Hb level is >11.5 or has increased ≧1.0 g/dL for two consecutive weeks from the mean Hb level during the Run-In Phase (1202), the EPO GMMOs dose may be reduced by removing or ablating or inactivating one or more of the implanted EPO GMMOs. The target of the reduced dose will be a dose decreased up to 25% less than the original dose, and will be based on the ex-vivo assessment of EPO production by the EPO GMMOs prior to implantation.

Dose reduction in EPO GMMO subjects may additionally occur at any time in the setting of 3 consecutive weekly Hb measurements above 12.0 g/dL (calendar duration of approximately 2 weeks of Hb>12.0 g/dL). In these subjects the effective administered dose may be reduced by excision of one or more EPO GMMOs to decrease the total administered dose based upon ex-vivo assessment of EPO production by approximately 25-50%.

Dose reduction by further removing or ablating or inactivation EPO GMMOs, each time by up to 50%, can subsequently be performed in subjects who continue to have consecutive Hb>12.0 g/dL with at least 2 calendar weeks between EPO GMMOs removal or ablation or inactivation. If Hb is ≧13.0 g/dL on any single assessment, a further assessment will be repeated at the next dialysis session; and if Hb remains ≧13.0 g/dL, one or more EPO GMMOs may be removed or ablated or inactivated to achieve a dose reduction (based on in vitro assessment) of up to 50%.

Dose addition of further EPO GMMOs may be carried out by use of cryo-preserved MOs that may be thawed, genetically modified and implanted according to the methods of this invention.

### EXAMPLES

### Materials and Equipment List

Production medium was used to grow dermal micro-organs and comprises DMEM-F12 (HyClone cat# SH30023.02) supplemented with 2.5 μg/ml Amphotericin B (Gilead Sciences Inc., AmBisome®) and 50 μg/ml Gentamycin sulfate (Teva Pharmaceutical Industries, IKA-injection).

### Harvesting of Dermal Micro-Organs

Human dermal micro-organs (“DMO”) were harvested from an area of skin from a donor's lower abdomen (tummy tuck skin). Details of harvesting DMO are found in US-2013-0090669-A1 and WO 2013/118109, which are herein incorporated in their entirety.

### Viral Vectors

Helper-Dependent non-replicating adenoviral hEPO vectors used to transduce DMO included: HDAd-CAG-wt-hEPO (FIG. 1; SEQ ID NO: 14); HDAd-CAG-opt-hEPO (FIG. 2; SEQ ID NO: 16); HDAd-MAR-EF1α-opt-hEPO (FIG. 3; SEQ ID NO: 10) The vector backbone of HDAd-MAR-EF1α-opt-hEPO is CpG free; and HDAd-MAR-CAG-opt-hEPO-WPRE (FIG. 4; SEQ ID NO: 12).

AAV hEPO vectors used to transduce DMO included AAV-LK19-CAG-opt-hEPO-WPRE (SEQ ID NO:26); and AAV-LK19-MAR-EF1alpha-opt-hEPO (SEQ ID NO:27). Additional vectors are described in the Figures and Examples.

Analysis of hEPO Concentrations

ELISA Kit systems from R&D Systems Inc., Quantikine® IVD Human Erythropoietin ELISA, cat# DEP00-10 were used to determine erythropoietin (hEPO) concentrations following in vitro expression (culture medium) and in vivo expression (serum) of hEPO from GMMOs.

### Human EPO Expression Cassettes

CAG-wtEPO: The CAG-wtEPO expression cassette present within the HDAd or AAV vector is schematically illustrated in FIG. 1. The CAG-wtEPO expression cassette encodes a wild-type (wt) erythropoietin polypeptide (EPO) (SEQ ID NO: 3). The nucleic acid sequence of the CAG-wtEPO expression cassette is presented as SEQ ID NO: 17. The CAG-wtEPO includes a CAG promoter sequence (SEQ ID NO: 7); human EPO intron-less gene from ATG to the stop codon (SEQ ID NO: 1); and SV40 poly A sequence (SEQ ID NO: 9).

CAG-opt-EPO:

The CAG-optEPO expression cassette present within the HDAd or AAV vector is schematically illustrated in FIG. 2. The CAG-opt-EPO expression cassette encodes an optimized erythropoietin polypeptide (EPO) (SEQ ID NO: 2). The nucleic acid sequence of the CAG-opt-EPO expression cassette is presented as SEQ ID NO: 15. The CAG-opt-EPO includes a CAG promoter sequence (SEQ ID NO: 7); optimized human EPO intron-less gene from ATG to the stop codon (SEQ ID NO: 2); and SV40 poly A sequence (SEQ ID NO: 9). The optimized human EPO gene was optimized for human codon usage.

MAR-Opt-hEPO-WPRE (Also Termed MAR-CAG-Opt-hEPO Herein):

The MAR-opt-hEPO-WPRE expression cassette present within the HDAd vector is schematically illustrated in FIG. 4. The MAR-opt-hEPO-WPRE expression cassette encodes an optimized erythropoietin polypeptide (EPO) (SEQ ID NO: 2). The nucleic acid sequence of the MAR-opt-hEPO-WPRE expression cassette is presented as SEQ ID NO: 13. The MAR-opt-hEPO-WPRE includes a human IFNβ S/MAR regulatory sequence (SEQ ID NO: 5); a CAG promoter (SEQ ID NO: 7); optimized human EPO intron-less gene from ATG to the stop codon (SEQ ID NO: 2) WPRE regulatory sequence (SEQ ID NO: 8); and SV40 poly A sequence (SEQ ID NO: 9). The optimized human EPO gene was optimized for human codon usage.

MAR-EF1α-opt-hEPO:

The MAR-EF1α-opt-hEPO expression cassette present within the HDAd or AAV vector is schematically illustrated in FIG. 3. The HDAd or AAV vector backbone may be CpG free. The MAR-EF1α-opt-hEPO expression cassette encodes an optimized erythropoietin polypeptide (EPO) (SEQ ID NO: 2). The nucleic acid sequence of the MAR-EF1α-opt-hEPO expression cassette is presented as SEQ ID NO: 11. The MAR-EF1α-opt-hEPO includes a CpG free human β-globin MAR regulatory sequence (SEQ ID NO: 6); an EF1α promoter (SEQ ID NO: 18); optimized human EPO intron-less gene from ATG to the stop codon (SEQ ID NO: 2); SV40 poly A sequence (SEQ ID NO: 9); and human IFNβ S/MAR regulatory sequence (SEQ ID NO: 5). The optimized human EPO gene was optimized for human codon usage.

### Preparation of Genetically Modified Dermal Micro-Organs Expressing Erythropoietin (EPO GMMO)

Preparation of hEPO GMMOs was carried out according to in-house Standard Operating Procedures (SOPs). Briefly, the DMOs were harvested from a human subject as described in US-2013-0090669-A1, which is herein incorporated in its entirety. The DMOs were washed once by saline followed by three washes with production medium not containing serum. Alternatively, in certain instances serum was included in the media.

The rinsed DMOs intended for the production of hEPO GMMOs were divided into different groups (8-10 DMOs/group) classified according to the viral vector to be used for transduction:
Group 1: HDAd-CAG-wtEPO;
Group 2: HDAd-MAR-CAG-opt-hEPO-WPRE;
Group 3: HDAd-MAR-EF1a-opt-hEPO;
Group 4: HDAd-CAG-opt-hEPO;
Group 5: AAV-LK19-MAR-CAG-opt-hEPO-WPRE; and
Group 6: AAV-LK19-MAR-EF1a-opt-hEPO.

The DMOs of each group were incubated individually in 1 ml production medium in a 5% CO₂, 32° C. incubator for 24 hours. Following incubation, the DMOs of each distinct group were transduced with 250 μl of production media containing 1.5×10¹⁰ Helper-Dependent Non-Replicating adenoviral (HDAd) hEPO viral particles of the respective treatment group. HDAd vectors lack all viral protein coding sequences and contain only the cis-acting elements required to replicate and package the vector DNA. The mixture was placed on an orbital shaker for 4 hours (300 rpm) followed by 20 hours incubation with no shaking in a 5% CO₂, 32° C. incubator. For terminating transduction step, the solution containing the viral vector was removed and discarded, and three consecutive medium exchanges with 3 ml of fresh production medium were performed in order to remove residual, unabsorbed viral particles. Following the washes, hEPO GMMOs were incubated in a 24 well plate (1 BP/well) filled with 1 ml of fresh production media and incubated in a 5% CO₂, 32° C. incubator for 3 days. The medium of each hEPO GMMO group was exchanged again after 3 days and the spent medium was collected to assay hEPO levels measured using ELISA system.

For EPO GMMOs used in the treatment of human subjects, the manufacturing process is performed under good manufacturing practice (GMP) conditions, and includes sequential processes to yield the final product. All DMOs are harvested from the skin on the lower abdomen. Ten dermal core tissue samples, approx. 30 mm in length and 2 mm in width, are typically removed in an ambulatory procedure room at the clinical center using a harvesting device with a 14-gauge coring needle under local anesthesia. Sequential processing includes but is not limited to: harvesting DMOs from subjects, introducing DMOs in culture, transducing DMOs with HDAd-EPO vector of choice to create EPO GMMOs, product release following characterization, secretion level measurements and sterility tests, and implanting EPO GMMOs into subjects.

### Ex Vivo Micro-Organ Maintenance

Every 3-4 days, used production media was collected, and the level of the secreted recombinant protein were assessed along with the viability of the hEPO GMMOs. Fresh Production media was added to the 24-well plate.

### In Vivo Animal Studies

Eight week old (at day of implantation) male SCID mice (severe combined immunodeficiency mice NOD.CB17-Prkdcscid/NCrHsd from Harlan Laboratories) were used in the SCID mouse studies.

Administration of hEPO GMMOs to SCID Mice

The hEPO GMMOs obtained from each treatment group were implanted subcutaneously in the back of 4-5 individual mice using 10G implantation needles (two hEPO GMMOs per each mouse).

In order to measure concentrations of human EPO (hEPO) in blood and hematocrit levels, mouse blood was collected (100 μl) at time points as follows: a few days before hEPO GMMO implantation (baseline), six days post hEPO GMMO implantation, and then approximately every 10 days for the duration of the experiment. Hematocrit levels were measured by the centrifugation method and serum hEPO levels were measured using the ELISA kit system, according to manufacturer protocols.

### Glucose Measurements

Glucose metabolism is used as a non-destructive assay to determine in vitro genetically modified micro-organ viability. Tissue glucose consumption was evaluated using either Sigma-Aldrich Corporation's GAGO20 Glucose (GO) Assay Kit, according to manufacturer's instructions and/or a Glucose Meter (Accu-Check Sensor/Performa, Roche or equivalent).

### Hematocrit Measurements

Hematocrit levels were assayed using centrifugation using the reference method recommended by The National Committee for Clinical Laboratory Standards (NCCLS), as is known in the art. To determine the hematocrit, whole blood in a tube was centrifuged at 10-15,000×g for 5 minutes to pellet the red cells (called packed erythrocytes), and the ratio of the column of packed erythrocytes to the total length of the sample in the capillary tube was measured with a graphic reading device within 10 minutes of centrifugation.

Administration of hEPO GMMOs to Humans

The hEPO GMMOs implantation procedure is performed around day 9 following the DMO harvesting procedure. Genetic modification, using HDAd-MAR-EF1α-opt-hEPO or HDAd-CAG-MAR-opt-hEPO-WPRE, is performed ex-vivo and the GMMOs are evaluated for hEPO secretion as described above. The implantation procedure occurs on a non-hemodialysis day. The number of hEPO GMMOs to be implanted will be determined based on the target dose and the ex-vivo determination of GMMO potency.

EPO GMMOs will be implanted SC in the abdomen [with an implantation devise described in WO2013/118109, which is incorporated here in full], under local anesthesia in an ambulatory procedure room at the clinical center.

Subjects receive their assigned hEPO GMMO produced hEPO dose based upon the ex-vivo measured GMMO hEPO secretion.

The hEPO GMMOs were implanted subcutaneously in an ambulatory procedure room at the clinical center. Subjects received IV prophylactic antibiotics (1 gram cephalosporin or the antibiotic of choice by treating physician) and local anesthesia including lidocaine and epinephrine.

At the time of implantation of each GMMO, Depo-Medrol® was administered subcutaneously around each GMMO implant site. Note that the injection sites should be as close as possible to the GMMO and no more than 5 mm away from the GMMO.

Depo-Medrol injections regimen: Do not inject on or too close to the GMMO (less than 1 mm). Use an insulin syringe of 0.5 cc. Disinfect the skin around the GMMO implantation site with 70% ethanol or Chlorhexidine. Injection must be done subcutaneously, do not pinch the skin in the injection area. Inject twice on each side of the GMMO, insert the needle first at the proximal end of the GMMO and infiltrate, and then again at the middle of the GMMO and infiltrate. Inject 8 mg per GMMO of Depo-Medrol 40 mg/ml. this means total of 0.2 cc per GMMO. Inject 0.1 cc on each side of the GMMO in 2 injections of 0.05 each.

Following implantation for all hEPO GMMO subjects, if the Hb response is found to be above the acceptable range a phlebotomy can be performed to reduce Hb levels and if not effective then the hEPO GMMOs dose will be reduced by excision of one or more hEPO GMMOs.

### Example 1

### EPO GMMOs In Vitro hEPO Secretion Reproducibility Using Different Skin Samples

Objective:

This study tested whether similar in-vitro GMMO secretion profiles are obtained when using different constructs to transduce MOs harvested from three different tummy-tuck donated skins.

Experimental Procedure in Brief:

DMOs were harvested from three different skin samples (HA-131, HA-132, HA-138) obtained from healthy donors. The experimental groups consisted of 4 DMOs that were transduced with HDAd-CAG-wt-hEPO or with the HDAd constructs comprising additional regulatory sequences, e.g., MAR, at 1.5×10¹⁰ vp/BP. GMMOs were maintained in production media supplemented with 10% DBS for the duration of the experiment. Other parameters such as well plates, media volume, and incubation conditions (32° C., 5% CO₂) remained unchanged for the entire experiment. Culture media was replaced every 3-4 days, collected and hEPO levels were tested by ELISA.

In a separate experiment EPO GMMOs were prepared as follows: EPO GMMOs transduced with the different vectors were prepared. Production media containing the secreted hEPO from four individual GMMOs of each treatment group were collected, pooled and analyzed for their hEPO concentration by ELISA (R&D Systems Inc., Quantikine® IVD Human Erythropoietin ELISA, Cat# DEP00-10). The hEPO values ranged from 2457 to 5800 IU/ml, as indicated in Table 1 below. Samples were prepared and analyzed before and after passage on an anti-EPO column. The column used was a thin monolith with immobilized monoclonal anti-EPO antibody 3F6 which has a high affinity to both endogenous and recombinant human EPO (Art. No. 0250 EPO purification kit, Maiia Diagnostics, Uppsala, Sweden).

TABLE 1
Samples description

| | | EPO | |
|---|---|---|---|
| Sample | Sample | Concentration | |
| Number | Identification | IU/BP/ml | Sample Source |
| 1 | H-185 wt-EPO | 5548 | Tummy Tuck ID#569 |
| 2 | H-185 opt-EPO | 8642 | Tummy Tuck ID#569 |
| 3 | H-173 wt-EPO | 2457 | Tummy Tuck ID#566 |
| 4 | H-173 opt-EPO | 5550 | Tummy Tuck ID#566 |
| 5 | H-176 wt-EPO | 5700 | Tummy Tuck ID#558 |
| 6 | H-176 opt-EPO | 4500 | Tummy Tuck ID#558 |

References samples used for isoelectric focusing:

1) Biological Reference Preparation (BRP batches 1 and 2a) from the European Pharmacopoeia Commission (an equimolecular mixture of rHuEPO: epoetin a and b) and darbepoetin a (NESP) from Amgen were mixed and systematically included in the IEF runs so that they were present in all of the analyzed images as reference position markers.

2) Human urinary EPO preparation from the National Institute for Biological Standards and Control (NIBSC).

Isoelectric Focusing—

The isoelectric profiles of the hEPO samples were monitored using the IEF and “double blotting” methods. Samples and references were loaded onto a polyacrylamide gel (5% total concentration of acrylamide and bisacrylamide, and 3% cross-linker) with a pH gradient of 2-8, 7M urea, 5% (w/v) sucrose, and thickness of 1 mm. Samples were run on the gels according to the following protocol: pre-focusing at 250V at 10° C. for 30 minutes followed by a focusing step at 1 W/cm of the gel length, and finally a stop at 3600 V/h.

Double Immunoblotting:

First semi-dry blotting was performed using transfer buffer (25 mM Tris, 192 mM glycine) at 1 mA/cm2, for 30 min followed by 45 min incubation in 5 mM DTT at 37° C., and then the membrane was blocked for 45 min in PBS containing 5% (w/v) non-fat milk. Immunoblotting was performed by incubating the blocked membrane for 1 hour with mouse anti-Human EPO (AE7A5) antibody diluted in 1% (w/v) non-fat milk/PBS solution. A second semi-dry blotting was performed using 50 mM glycine/HCl, 0.1M NaCl, pH 2.6 solution, at 1 mA/cm2 of the membrane for 10 min.

At the end of this step a second blocking was performed again as described above. Following the second blocking, the membrane was incubated overnight at 4° C. with biotinylated secondary antibody (goat anti-mouse IgG (H+L)) diluted in 1% (w/v) non-fat milk/PBS solution. In order to visualize the blotted EPO, the membrane was incubated for 45 min at room temperature with a streptavidin-peroxydase diluted in 1% (w/v) non-fat milk/PBS solution. A substrate was then added, 3,3′-Diaminobenzidine (DAB) Tetrahydrochloride tablets (cat# D5905, Sigma), and a chemiluminescent reaction was developed in which its intensity was captured using a CCD camera.

### Results

FIG. 6 results suggest that skin source has an effect on different constructs' performance, however, in all cases the constructs comprising additional regulatory elements, e.g., MAR, performed at least as well as the control HDAd-CAG-wt-hEPO construct. In the second experiment, when skin HA-132 was transduced, no significant difference in secretion profiles was observed. However, when skins HA-131 and HA-138 were transduced with the three different vectors, secretion levels of the constructs, HDAd-MAR-CAG-opt-hEPO-WPRE and HDAd-MAR-EF1α-opt-hEPO, were about 30-40% higher than the current HDAd construct.

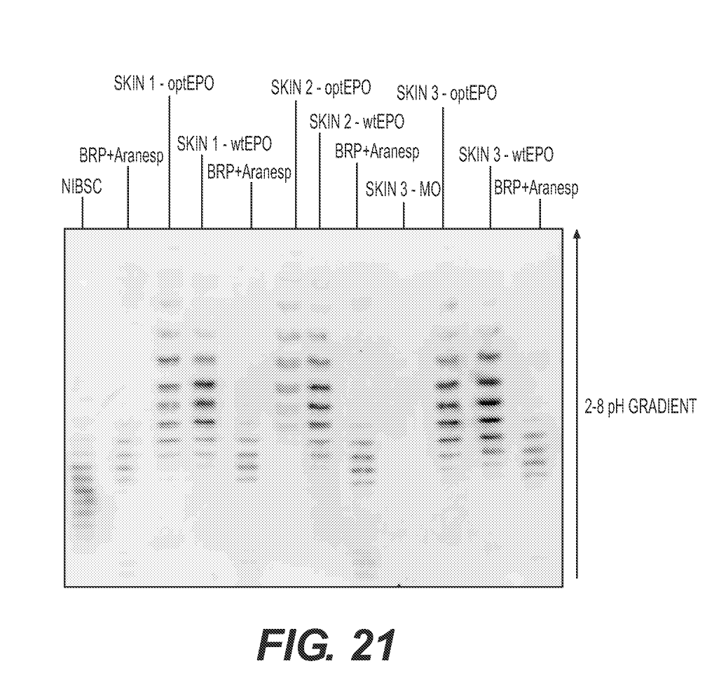

FIG. 21 represents hEPO isoelectric focusing results obtained from 3 skins transduced with the HDAd-CAG-wt-hEPO or HDAd-MAR-EF1α-opt-hEPO vectors. As can be seen a similar isoelectric patterns were observed for each skin, indicating similar hEPO isoforms, even though transduced with the two different vectors. As expected, sample taken from untransduced MO did not show any hEPO signal, validating the specificity of the method.

In comparison to the pH of the recombinant and human urinary derived standards (pH range of 2-4), the IEF of the tested samples was more basic (pH range of 3-7). These results are in agreement with the results obtained from samples collected from different human subjects (above). Moreover, the results suggest that in contrast to recombinant EPO proteins, the EPO produced by hEPO GMMOs is patient specific and is closer in composition to the endogenous EPO.

### Example 2

### In-Vitro Performance of GMMOs Transduced with HDAd-CAG-Wt-hEPO and Expression Cassettes Comprising Additional Regulatory Sequences

Objective:

This study tested whether different in-vitro performance is obtained when using the different constructs to transduce and process GMMOs. The level of target protein secretion and duration of expression as reflected by EPO secretion to the media was used to evaluate the different constructs performance.

Experimental Procedure:

GMMOs were prepared utilizing the standard operating procedures (SOPs) for GMMO production. In the current study each experimental group contained four DMOs which were harvested from the same abdominoplasty donated skin. Harvesting was done using a Nouvag medical drill set at 7,000 RPM equipped with a 14G coring needle and with a proprietary DermaVac positioning device. The DMOs were washed once by saline followed by five washes in production media (supplemented with 10% Defined Bovine Serum [DBS]) and then incubated individually in 1 ml production media in a 5% CO₂, 32° C. incubator for 24 hours. After this latency period, the MOs were transduced with 240 μl production media containing 1.5×10¹⁰ Vp/BP of one of the HDAd-EPO vectors. The mixture was placed on an orbital shaker for 4 hours (300 rpm) followed by 20 hours with no shaking in a 5% CO₂, 32° C. incubator. To stop transduction, the solution containing the viral vector was removed and discarded, and six media exchanges with 3 ml of fresh production medium were performed in order to remove residual, unabsorbed viral particles. Following the washes, 1 ml of media was added and the GMMOs were incubated in a 5% CO₂, 32° C. incubator for 3 days. After the media was exchanged, EPO levels were measured in the spent media by ELISA (R&D Systems Inc., Quantikine® IVD Human Erythropoietin ELISA, Cat# DEP00-10). The GMMOs were incubated for several additional months under identical conditions, with media exchanges every 3-4 days. EPO levels were initially measured every week, and gradually reduced in frequency to once every 10 days.

### Results

While comparing the secretion levels of the GMMOs transduced with the different constructs, it was shown that the HDAd-MAR-CAG-opt-hEPO-WPRE and HDAd-MAR-EF1α-opt-hEPO constructs had secretion profiles at least matching and in general greater than the secretion profile of the HDAd-CAG-wt-hEPO construct (FIG. 5). Moreover, the results obtained in this experiment suggest that the in-vitro secretion decay rate of the new constructs is more moderate: about 3 months from the initiation of the experiment the new constructs secrete about 50% more than the current construct.

### Example 3

### EPO GMMOs In Vivo hEPO Serum Levels of Implanted SCID Mice

Objective:

This study tested whether similar in-vivo GMMO secretion profiles are obtained in SCID mice serum post implantation of BPs harvested from tummy-tuck donated skin, which were transduced using the different viral constructs.

Experimental Procedure in Brief:

DMOs were harvested, transduced with the different HDAd vectors, and maintained for seven days in-vitro before their implantation into six-week old SCID mice. In-vitro hEPO levels secreted by each GMMO (“BP”) were determined by ELISA one day prior to their implantation into the mice. Five mice were implanted with two BPs transduced with HDAd-CAG-wt-hEPO, five mice with two BPs transduced with HDAd-MAR-CAG-opt-hEPO-WPRE and the last five mice with two BPs transduced with HDAd-MAR-EF1α-Opt-hEPO. The two BPs were implanted subcutaneously on the dorsal side of the mouse, one on each side, using 14G implantation needles. Baseline bleed: mice were bled 8 days prior to BP implantation in order to measure the background concentration of hEPO and hematocrit levels. Once implanted blood samples were collected every 10-14 days for 18 weeks in order to monitor in-vivo GMMOs performance. Hematocrit was measured by the centrifugation method and serum hEPO levels in the blood were measured by a hEPO ELISA kit according to the protocol suggested by the manufacturer.

### Results

FIG. 7a shows long term hEPO secretion levels in the serum of SCID mice implanted with HDAd-transduced GMMOs, as well as a rise in the corresponding hematocrit levels.

All mice showed a rapid elevation of serum hEPO levels and a corresponding increase in their hematocrit percentage, indicating that the implanted BPs are viable and can deliver bioactive levels of hEPO for an extended time period (since an increase of few mU in EPO serum levels may increase significantly SCID mice % hematocrit, no difference in hematocrit levels may be expected between the different treatments). Results obtained also show that constructs HDAd-MAR-CAG-optEPO-WPRE and HDAd-MAR-EF1α-optEPO improve GMMOs in-vivo secretion levels and decrease decay rate (FIG. 7b); at day 125 post implantation, 3.54% and 2.87% from initial peak levels were detected in the HDAd-MAR-CAG-opt-hEPO-WPRE and the HDAd-MAR-EF1α-opt-hEPO constructs, respectively. In contrast only 0.58% of the initial peak was measured when the vector HDAd-CAG-wt-hEPO was used. Moreover, while considering absolute secretion values, serum EPO levels remained 7-20 folds higher using HDAd-MAR-CAG-opt-hEPO-WPRE and the HDAd-MAR-EF1α-opt-hEPO constructs compared to HDAd-CAG-wt-hEPO from day 96 and on post implantation. When un-transduced MOs were implanted as a control, no hEPO rise or change in hematocrit percentage was observed (data not shown).

### Example 4

### Methylprednisolone (Depo-Medrol®) Administration Decreased Decay Rate of hEPO in Serum of EPO GMMO Implanted SCID Mice

In an attempt to enhance in-vivo performance, while reducing inflammation and improving hEPO GMMO integration post implantation, the steroid Depo-Medrol® was injected at the mouse implantation site once a week. This steroid (Depo-Medrol®) is clinically approved and routinely used in plastic surgeries to reduce inflammation and improve integration and healing of the surgical wound. Each Depo-Medrol application was done as follows: 100 μl solution containing 1 mg of Depo-Medrol is injected surrounding each implanted GMMO. EPO-expressing GMMOs were prepared and implanted as described above. Two EPO GMMOs were implanted per SCID mouse.

### Results

As seen in FIG. 8a and FIG. 8b, when Depo-Medrol® was injected to GMMO implanted mice, GMMO secreted human EPO decay rate from initial peak serum level was reduced and hEPO secretion duration was improved. For example, Day 20 post implantation, 35% from serum hEPO concentration at peak level was recovered in the Depo-Medrol® group in comparison to only 11% of the control group.

### Example 5

### Different Dose Regimes of Depo-Medrol® Decrease hEPO Serum Decay Rate on EPO GMMO Implanted SCID Mice

Objective:

This study tested whether similar in-vivo GMMO secretion profiles are obtained in SCID mice serum post implantation of BPs transduced with the different viral constructs and when Depo-Medrol is applied to the implantation site every week.

Experimental Procedure in Brief:

DMOs were harvested, transduced with the different HDAd vectors, and maintained for seven days in-vitro before their implantation into six-week old SCID mice. In-vitro hEPO levels secreted by each BP were determined by ELISA one day prior to their implantation into the mice. Five mice were implanted with two BPs transduced with HDAd-CAG-wt-hEPO, five mice with two BPs transduced with HDAd-MAR-CAG-opt-hEPO-WPRE and the last five mice with two BPs transduced with HDAd-MAR-EF1a-Opt-hEPO. The two BPs were implanted subcutaneously on the dorsal side of the mouse, one on each side, using 14G implantation needles. On the day of implantation and once a week thereafter, 1 mg of Depo-Medrol® (in 100 μl saline solution) was injected subcutaneously around the two implanted GMMOs of the HDAd-MAR-CAG-opt-hEPO-WPRE and HDAd-MAR-EF1α-Opt-hEPO constructs (the control group, HDAd-CAG-wt-hEPO, did not receive any steroid treatment). Baseline bleed: mice were bled 5 days prior to BP implantation in order to measure the background concentration of hEPO and hematocrit levels. Implanted SCID's blood samples were collected every 10-14 days for 16 weeks in order to follow in-vivo GMMOs performance. Hematocrit was measured by the centrifugation method and serum hEPO levels in the blood were measured by a hEPO ELISA kit (see above) according to the protocol suggested by the manufacturer.

Additional compounds administered to test enhanced in vivo hEPO GMMO performance included: Vitamin C, N-Acetyl Cysteine, Caspase-1 Inhibitor (Z-Wehd-Fmk), Cytosine, Pirfendone, Tempol, Cathepsin B inhibitor (CA-074-OME), Demecolcine, zVAD (pan caspase inhibitor), Minocycline hydrochloride (caspase 1 and 3 inhibitor), Actemra (IL-6 inhibitor), Aspirin (cox inhibitor), MIF antagonist (macrophage migration inhibitory factor), Infliximab (Anti TNF), Mitomycin C, Resveratrol, and Hyaluronic Acid (data not shown). Each test compound was administered into different SCID mice groups.

### Results

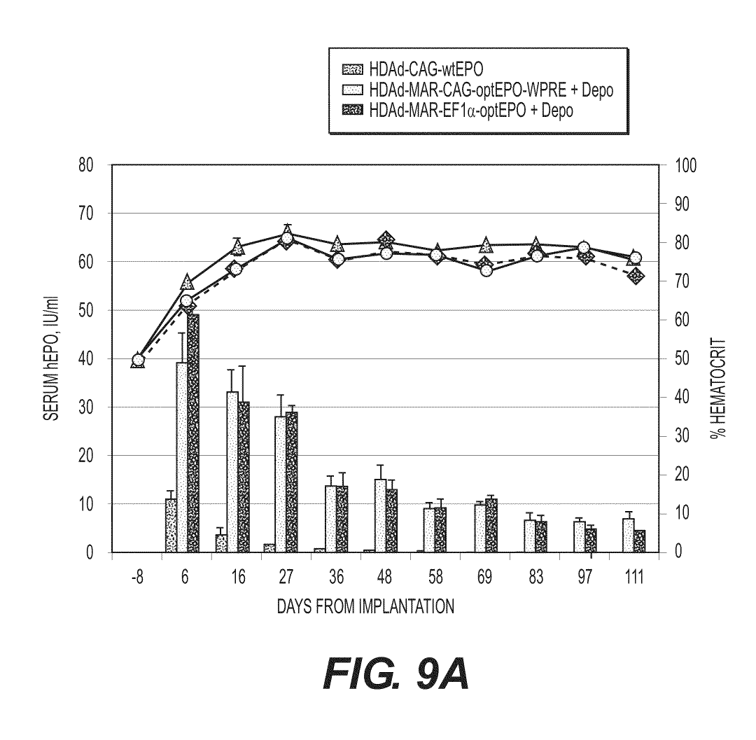

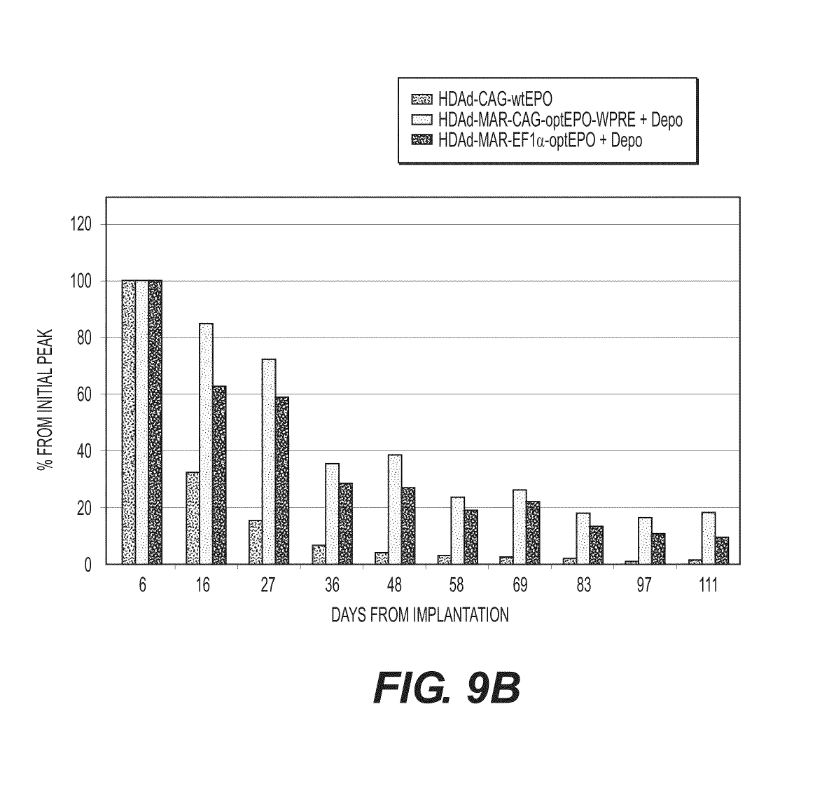

FIG. 9a shows long term hEPO secretion levels in the serum of SCID mice implanted with HDAd-transduced GMMOs, as well as a rise in the corresponding hematocrit levels.

All mice showed a rapid elevation of serum hEPO levels and a corresponding increase in their hematocrit percentage, indicating that the implanted BPs are viable and can deliver bioactive levels of hEPO for an extended time period. Moreover, results obtained also show that in addition to the improved performance due to the use of the HDAd-MAR-CAG-opt-hEPO-WPRE and HDAd-MAR-EF1α-Opt-hEPO constructs (FIGS. 7a and 7b), Depo-Medrol® application to the implantation site may further improve HDAd-MAR-CAG-opt-hEPO-WPRE and HDAd-MAR-EF1α-opt-hEPO constructs transduced GMMOs in-vivo secretion levels and decrease the decay rate during sustained secretion (FIG. 9b). At day 111 post implantation, 18% and 9% from initial peak levels were detected in the HDAd-MAR-CAG-opt-hEPO-WPRE and the HDAd-MAR-EF1α-opt-hEPO constructs, respectively. In contrast only 0.8% from initial peak was measured when GMMOs transduced with the control vector, HDAd-CAG-wt-hEPO, were implanted and Depo-Medrol® was not applied to their implantation site. Moreover, while considering absolute secretion values, serum EPO levels remained 40-50 fold higher using HDAd-MAR-CAG-opt-hEPO-WPRE and HDAd-MAR-EF1α-Opt-hEPO constructs and Depo-Medrol compared to HDAd-CAG-wt-hEPO without Depo-Medrol from day 97 and on post implantation. When un-transduced DMOs were implanted as a control, no hEPO rise or change in hematocrit percentage was observed (data not shown). The testing of numerous different anti-inflammatory compounds, steroids, glucocorticoids and anti-oxidants injected at the site of hEPO GMMO implantation in the SCID mouse model surprisingly revealed that positive results, i.e., decreased serum human EPO decay rates and sustained EPO serum levels, were only observed with Depo-Medrol® injections. Results following injections of anti-caspase 1 did not provide decreased rate decay to the extent observed with Depo-Medrol® under the experimental conditions tested. Further, all other anti-inflammatory agents, anti-proliferative agents and anti-oxidants tested had no positive effect under the doses and application methods tested (data not shown).

### Example 6

### Correlation Between hEPO GMMO Administered Dose and Net Peak Increase in Serum hEPO Levels Above Base Line

A Phase I/II clinical trial were performed, in which pre-dialysis anemic patients with chronic kidney disease (CKD), stage III and stage IV, were implanted with autologous hEPO GMMOs transduced with HDAd-CAG-wtEPO vector. A single implantation treatment with hEPO GMMOs provided between 1.5 months to greater than two years of effective hEPO therapy. Patients were treated at 18-25 IU/kg/day (low dose), at 35-45 IU/kg/day (mid dose) or at 55-65 IU/kg/day (high dose).

### Results

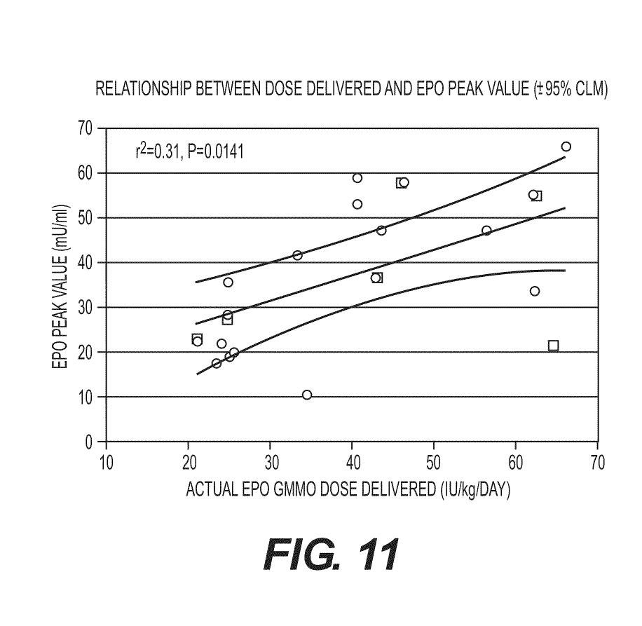

A dose response was demonstrated between the administered dose of daily hEPO production by hEPO GMMOs prior to implantation and peak serum concentration of hEPO in the treated patients after implantation (FIG. 11).

### Example 7

### In-Vivo SCID Mice Results Using New HDAd Constructs where Depo-Medrol® was Applied to the Implantation Site Every Second Week

Objective:

This study tested whether similar in-vivo GMMO secretion profiles are obtained in SCID mice serum post implantation of BPs transduced with the different viral constructs and with Depo-Medrol® applied to the implantation site every second week.

Experimental Procedure in Brief:

DMOs were harvested, transduced with the different HDAd vectors, and maintained for seven days in-vitro before their implantation into six-week old SCID mice. In-vitro hEPO levels secreted by each GMMO were determined by ELISA one day prior to their implantation into the mice. Five mice were implanted with two GMMOs transduced with HDAd-CAG-wt-hEPO, five mice with two GMMOs transduced with HDAd-MAR-CAG-opt-hEPO-WPRE and the last five mice with two GMMOs transduced with HDAd-MAR-EF1α-Opt-hEPO. The two GMMOs were implanted subcutaneously on the dorsal side of the mouse, one on each side, using 14G implantation needles. On the day of implantation and every second week afterwards, 1 mg of Depo-Medrol® (in 100 μl saline solution) was injected subcutaneously around the two implanted GMMOs of each SCID. Baseline bleed: mice were bled 5 days prior to GMMO implantation in order to measure the background concentration of hEPO and hematocrit levels. After implantation, blood samples were collected every 10-14 days for 17 weeks in order to follow in-vivo GMMOs performance. Hematocrit was measured by the centrifugation method and serum hEPO levels in the blood were measured by a hEPO ELISA kit according to the protocol suggested by the manufacturer.

### Results

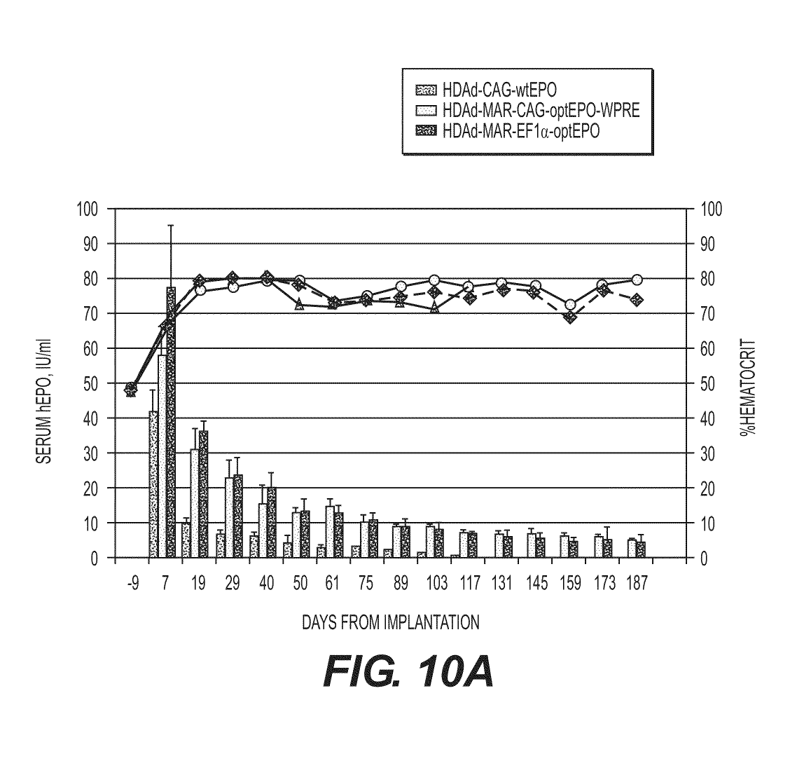

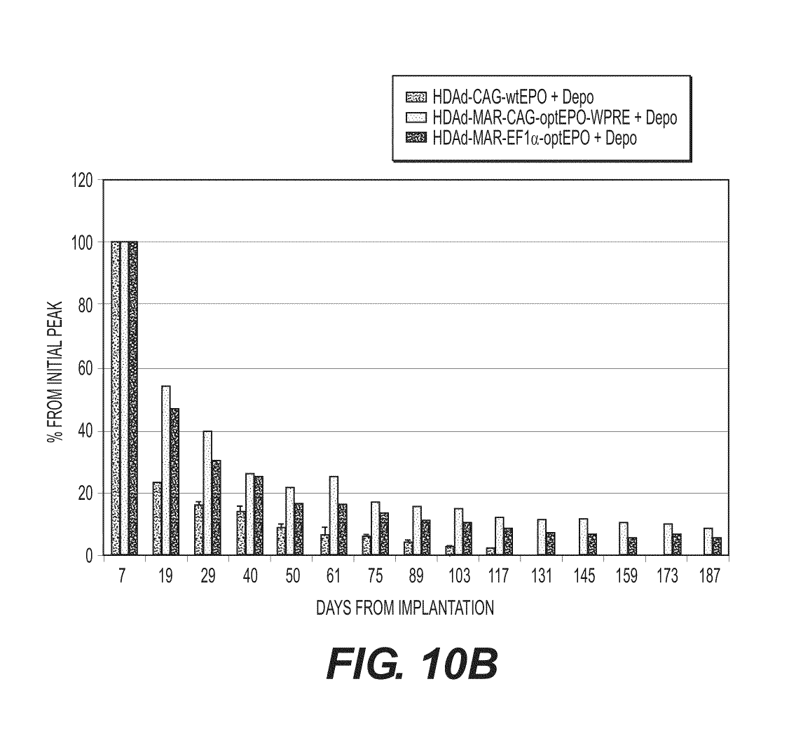

FIG. 10a shows long term hEPO secretion levels in the serum of SCID mice implanted with HDAd-transduced GMMOs, as well as a rise in the corresponding hematocrit levels.

All mice demonstrated a rapid elevation of serum hEPO levels and a corresponding increase in their hematocrit percentage, indicating that the implanted BPs are viable and can deliver bioactive levels of hEPO for an extended period of time. Results obtained also suggest that in addition to the improved performance due to the use of HDAd-MAR-CAG-opt-hEPO-WPRE and HDAd-MAR-EF1α-Opt-hEPO constructs (FIGS. 7a and 7b), Depo-Medrol® application to the implantation site may further improve all constructs transduced GMMOs in-vivo secretion levels and decreased the decay rate of the sustained delivery (FIG. 10b). At day 117 post implantation, 12% and 8% from initial peak levels were detected in the HDAd-MAR-CAG-opt-hEPO-WPRE and the HDAd-MAR-EF1α-opt-hEPO constructs, respectively. In contrast only 2% from of the initial peak was measured when GMMOs transduced with the control vector, HDAd-CAG-wt-hEPO, were implanted and Depo-Medrol® was applied every other week to their implantation site. In this experiment, Depo-medrol improved the control treatment group recovery by a factor of around 4, from 0.59% (see FIG. 7) to 2%; however, it was not close to the sustained performance observed using the HDAd-MAR-CAG-opt-hEPO-WPRE and HDAd-MAR-EF1α-Opt-hEPO constructs. In addition, data obtained in this experiment may suggest that Depo-Medrol application may be reduced from weekly to every second week. Moreover, while considering absolute secretion values, serum EPO levels remained 6-7 folds higher using HDAd-MAR-CAG-opt-hEPO-WPRE and HDAd-MAR-EF1α-Opt-hEPO constructs compared to HDAd-CAG-wtEPO from day 103 and on post implantation. When un-transduced DMOs were implanted as a control, no hEPO rise or change in hematocrit percentage were observed (data not shown).

Conclusions using constructs HDAd-MAR-CAG-opt-hEPO-WPRE, HDAd-MAR-EF1α-Opt-hEPO and HDAd-CAG-wtEPO, and administration of methylprednisolone (Examples 1-5 and 7):

Constructs HDAd-MAR-CAG-opt-hEPO-WPRE and HDAd-MAR-EF1α-opt-hEPO had at least as good in-vitro secretion profile as the HDAd-CAG-wt-hEPO vector. However, the HDAd-MAR-CAG-opt-hEPO-WPRE and HDAd-MAR-EF1α-opt-hEPO constructs additionally showed improved in-vitro secretion levels and duration of secretion.

Skin source may have some effect on the different constructs' performance, however, in all of this study's observations, the HDAd-MAR-CAG-opt-hEPO-WPRE and HDAd-MAR-EF1α-opt-hEPO constructs performed at least as good, if not better, that the HDAd-CAG-wt-hEPO construct.

HDAd-MAR-CAG-opt-hEPO-WPRE and HDAd-MAR-EF1α-opt-hEPO significantly improved GMMO in-vivo performance compared with HDAd-CAG-wt-EPO while increasing secretion levels and, more importantly, the duration of secretion.

Depo-Medrol® application to the implantation sites further improved in-vivo performance for all constructs.

### Example 8

### Safety and Efficacy of Sustained EPO Therapy of Anemia in Chronic Kidney Disease Patients and End-Stage Renal Disease (ESRD) Dialysis Patients Using hEPO GMMO

Applicant has conducted a Phase I/II, prospective, open label, dose escalation study to evaluate safety and efficacy of the EPO GMMO of the invention.

The entire hEPO GMMO production process, from harvest to implantation, takes about 9-15 days. DMOs were harvested from dermis tissue in the lower abdomen, transduced with an HDAd-MAR-EF1α-optEPO vector, and hEPO GMMOs were re-implanted in the same general area.

The MAR-EF1α-optEPO vector construct used to transduce the micro-organs used in this clinical trial is shown in SEQ ID NO: 10. The transduced EPO GMMOs re-implanted to the subject comprise the nucleic acids of SEQ ID NO: 11.

The hEPO GMMOs were implanted at a dose of about 25 IU/Kg/day.

This human clinical trial involved autologous dermal GMMOs providing a novel recombinant protein production and delivery system capable of providing sustained secretion of therapeutic human EPO within the body, using a small tissue explant from the patient's own skin referred to as dermal micro-organs (DMOs).

Study Objectives: The objectives of this study were to assess safety and to evaluate the biologic activity of hEPO GMMO treatment. Biological activity assessments included duration of hEPO GMMO secretion as measured by serum EPO levels above baseline (baseline defined as mean serum EPO in screening, pre-harvesting, and pre-implantation visits).

Study Population: The study was conducted with humans aged 18 to 80 years diagnosed with anemia due to end stage renal disease on dialysis treatment for at least 6 months or due to chronic kidney disease (CKD).

Patients were enrolled in cohorts according to Table 2.

TABLE 2

| Group | Treatment | N |
|---|---|---|
| A | hEPO GMMO 18-25 IU/kg/day | 3 + 3 |
| B | hEPO GMMO 35-45 IU/kg/day | 3 + 3 |
| C | hEPO GMMO 55-65 IU/kg/day | 3 + 3 |

One objective was maintaining Hb levels within the target range of 9-11 g/dl or 9-12 g/dl or less than 12 g/dl.

Each patient received autologous hEPO GMMO tissue implants intended to provide sustained production and delivery of therapeutic levels of EPO for as much as six (6) months or more following hEPO GMMO implantation. Study periods were as follows in Table 3.

TABLE 3

| Period | | |
|---|---|---|
| No. | Period Name | Duration |
| I | Screening | |
| II | Run in Phase and Harvest and | 4 weeks from enrolment to |
| | hEPO GMMO Production | Implantation (day 0) |
| III | Implantation and Efficacy | Day 0 to 52 weeks |
| | Phase | |
| IV | Safety Follow-up | 6 months as of week 53 or |
| | | from early discontinuation |
| V | Long term safety follow up | up to 2 years |
| | (optional) | |

Timeline is relative to the day of implantation (Day 0).

### Period I: Screening Period

If the subject's transferrin saturation was less than 20% and ferritin less than 100 ng/ml, the subject was placed on a regimen of parenteral iron supplements no less than 500 mg and afterwards hematology parameters were retested. If transferrin saturation was then found higher than 20% and/or ferritin higher than 100 ng/ml, the subject was eligible to proceed for final decision for enrollment in the study, otherwise additional iron was administered as required. Patients treated with Acetyl Salicylic Acid (ASA) up to 325 mg/d were temporarily discontinued one week prior to harvest.

### Period II: Run in Phase and Harvest and EPO GMMO Production Period

The run-in phase was 4 weeks. On the 2^{nd}/3^{rd} week of the run-in, the harvest procedure was performed and in the 3^{rd}/4^{th} week of the run-in the implantation procedure was performed.

Subjects maintained a stable dose of rHuEPO+/−25% for the duration of the Run-In Phase. Subjects underwent weekly assessments on the second hemodialysis session of the week (or on a set day of the week for a clinic visit for CKD patients). On the first week EPO PK was taken pre dialysis, 15 minutes after injection of rHuEPO, 1 hour after injection of rHuEPO, at completion of hemodialysis session (defined as time when arterial line is disconnected), 18-24 hours after completion of hemodialysis session, and daily thereafter. During this week on dialysis days EPO PK was taken pre Dialysis and 15 minutes post injection of rHuEPO; for CKD patients on the first week EPO PK was taken on the first day of the week pre injection of EPO and 15 minutes 1 h, 4 h, and 18-24 h post injection and daily thereafter. During this week, on days rHuEPO is injected, EPO PK serum samples was taken pre and 15 minutes post rHuEPO injection. Weeks 2 till 4 EPO PK Samples were taken for ESRD on every dialysis day of the week prior to Dialysis and 15 minutes after rHuEPO injection and for CKD patients up to 3 times a week, on days rHuEPO was injected EPO PK was taken pre and 15 minutes post rHuEPO injection. Hematology was taken once a week, for ESRD patients it was taken on the second dialysis day of the week.

Subjects underwent the harvest procedure of up to 15 micro-organs (MOs) around the beginning of week 3 (some as early as week 1). The MOs were processed into hEPO GMMO according to the subject's group assignment. Implantation of the calculated dose of hEPO GMMO took place on a non-dialysis (for ESRD) day during week 4 (some earlier, as early as week 2).

The entire hEPO GMMO production process, from harvest to pre-implantation, took about 9-14 days.

Blood tests were taken 1-2 days prior to the implantation visit as well as on implantation day. The results were used in baseline determination of Hematology and erythropoietin values. A sample for testing anti-EPO antibodies and anti-adenovirus antibodies was also taken at this time. Heparin was not administered on the dialysis session prior to the Harvest visit.

At the harvesting visit, prior to the procedure, prophylactic antibiotics and local anesthesia was administered. Up to 15 dermal core tissue samples, approx. 30 mm long, were removed from the abdominal skin using a harvesting device with a 14 gauge coring needle under local anesthesia in an ambulatory operating room located at the treatment center. The skin donor sites were evaluated based on the Draize score, photographed and bandaged, and the subject was monitored for up to two (2) hours following harvest. Acetyl salicylic acid (ASA) was not re-started until after implantation.

The dermal samples were transferred in a sterile manner to a GMP production facility. As a key step in the GMP tissue processing protocol, the dermal cores were exposed one day after harvesting to a GMP-produced non-integrating HDAd viral vector for approximately 24 hours, to introduce the EPO gene and its associated expression cassette into the cells of the intact dermal tissue ex vivo, enabling them to produce and secrete human EPO. Subsequent washing and media changes were employed over the ensuing seven (7) days to reduce the residual viral vector titer to a minimum, for product sterility testing, and for measurement of EPO secretion.

Average EPO production by each hEPO GMMO prior to implantation was used to determine the number of hEPO GMMOs to implant in the subject in order to reach the desired total dose per kilo per day. Patients continued rHuEPO treatment until day −1 or earlier. From Implantation day (Day 0), rHuEPO was not administered. Heparin was not administered on the dialysis session prior to the implantation visit. Blood samples were taken 1-2 days, for ESRD on the dialysis visit when connecting to dialysis, prior to the implantation visit as well as on implantation day. The results were used in baseline determination of hematology and erythropoietin values.

Overall, hematology and erythropoietin results from screening, pre-harvesting, and pre-implantation visits, were used to provide baseline values prior to start of hEPO GMMO therapy (baseline defined as mean serum EPO in screening, pre-harvesting, and pre-implantation visits).

### Period III (Implantation Efficacy Phase): Day 0 to Week 52

The hEPO GMMO implantation procedure was performed at visit 5 (Day 0), 9-14 days post harvesting, in an ambulatory operating room at a treatment center. For ESRD, implantation took place between 2 dialysis sessions, not on a dialysis day.

Prior to the procedure prophylactic antibiotics and local anesthesia were administered. Implantation of the desired number of hEPO GMMOs, based on the patient's target dose as per the group allocation, and the in-vitro secretion of each individual hEPO GMMO, was performed on the abdominal skin in an area other than where harvest was conducted, by intradermal or subcutaneous insertion using an implantation tool to control the depth of implantation, the implantation device and procedure are described in WO 2013/118109, which is incorporated here in full

Each patient was treated with SC injections of Depo-Medrol® (no less than 4 mg per hEPO GMMO implanted) at the implantation site immediately post implantation (a total of up to 40 mg Depo-Medrol®). In case of diabetic patients, blood glucose was monitored and glucose lowering treatment was adjusted, if needed, during the treatment with Depo-Medrol®.

The implantation sites were evaluated based on the Draize score and for hematomas, marked by tattooed dots at each end of each GMMO implantation site, photographed, applied with topical antibiotic and bandaged, and the subject was monitored for up to two (2) hours following implantation. Patients, who were treated with acetyl salicylic acid (ASA) prior to the harvest visit, will re-initiate it the day after the implantation.

During Period III, subjects were assessed by a clinical investigator as follows: EPO levels blood samples were drawn pre implantation and every day for the first week starting the day after implantation (on dialysis days samples were collected pre-dialysis). For the next 2-4 weeks, blood samples were taken pre-dialysis on every dialysis day, and for CKD patients, 3 times a week during clinic visits. From week 5 on, blood samples were taken once a week. For ESRD pre-dialysis on the second dialysis day in the week. Patients were evaluated by the clinical investigator once a week during the first 12 weeks. The same SC injections of Depo-Medrol® as given right after implantation (no less than 4 mg per GMMO implanted) was repeated every 2 weeks thereafter for a total of 8 weeks (total of 4 administrations of Depo-Medrol®). The second to fourth administrations of Depo-Medrol® were done on a dialysis day. In case of diabetic patients, blood glucose was monitored and glucose lowering treatment was adjusted, if needed, during the treatment with Depo-Medrol®.

Biochemistry including ferritin, iron, transferrin and its saturation, serum albumin, Vit B12, Folic Acid and PTH were taken as is routine at the dialysis unit. ESRD patients had their samples drawn pre-dialysis from the venous needle tubing when connecting to dialysis. Anti-Adenovirus antibodies were tested during this period at week 4 post implantation.

From week 13 onward, subjects were evaluated for study purposes by a clinical investigator every 2 weeks until week 24 and every one month thereafter. ESRD patients were evaluated on one of the days they visited the clinic for dialysis treatment. Complete physical examination took place on week 14 and week 26.

This Efficacy Phase (Period III) ends either when the patient requires exogenous EPO injections or at 52 weeks of treatment.

Table 4 shows a schematic of the clinical trial design.

TABLE 4

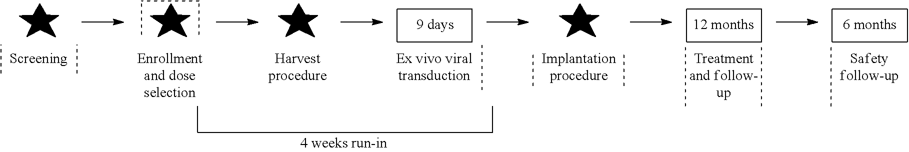

### Results

The clinical trial with the EPO GMMOs of the invention show surprising in vivo efficacy. See, FIGS. 15, 16, 17, and 18 and the corresponding Figure legends. The improved GMMOs described herein deliver sustained, physiologically relevant levels of endogenous erythropoietin (eEPO), which in turn maintains hemoglobin within a clinically desired range. Hemoglobin levels are maintained without the need for rescue rHuEPO or transfusion. The EPO GMMOs are safe and well-tolerated. Based on these results, the GMMOs described herein are useful in treating any of the diseases and disorders described herein, including end stage renal disease (ESRD), patients who are poorly responsive to recombinant humanized erythropoietin (rHuEPO), and Beta Thalassemia patients, including Beta Thalassemia Intermedia.

### Example 9

### Overview

In this study, the in-vitro and in-vivo performance of EPO GMMOs transduced with two types of viral vectors: Adeno-Associated Virus (AAV) and HDAd were studied. GMMOs were transduced with AAV-LK19-MAR-opt-hEPO-WPRE and HDAd-MAR-EF1α-opt-hEPO vectors and maintained in DMEM-F12 media supplemented with 10% serum. Analysis of in-vitro hEPO secretion showed that both HDAd and AAV transduced GMMOs secreted hEPO in vitro and in vivo.

Removal of serum from the in-vitro maintenance of both systems resulted in about a 40% reduction of hEPO secretion of the HDAd system, whereas a more significant reduction was measured for the AAV system (90%).

When GMMOs were transduced with AAV-MAR-CAG-opt-hEPO-WPRE or HDAd-MAR-EF1α-opt-hEPO vectors in the in-vivo SCID mice model, HDAd transduced GMMOs showed significantly higher hEPO serum levels. However, the AAV system showed a much more stable secretion profile for the duration of a few months.

Typically, harvested DMOs are converted to target protein secreting GMMOs in a several day in-vitro process. This manufacturing process includes a latency period following DMO harvesting, transduction with a viral vector and GMMO maintenance in production media. Another goal of this study was to assess the in vitro processing time to determine optimal timing.

DMOs were harvested, transduced with the HDAd-MAR-EF1α-opt-hEPO (SEQ ID NO: 27) or AAV-LK19-MAR-CAG-opt-hEPO-WPRE (SEQ ID NO: 26) viral vectors, and maintained for less than the standard 9 days in-vitro before their implantation into six week old SCID mice.

Growth medium 10% serum: HyClone DME/F-12 1:1 (xi)+2.50 mM L-Glutamine+15 mM HEPES Buffer (Thermo scientific, Cat# SH30023.01). Medium is supplemented with 10% DCS (HyClone Defined Bovine Calf Serum supplemented, Thermo scientific, Cat # SH30072.03); AmBisome 2.5 μg/ml (Amphotericin B Solution 250 ug/ml Biological Industries); Gentamycin sulfate 50 μg/ml (Gentamicin-IKA 80 mg/2 ml-Teva).

84 Dermal core MOs 30 mm were prepared in a sterile hood following the Clinical Harvesting Procedure Protocol with 14G needles (2.05 mm deep from skin surface) and back vacuum containing 2 ml of saline. The MO's were flushed out from the needles with saline. Needles were replaced every 4-5 harvest. The MO's were incubated for one minute in saline. Then all the MO's were washed 3 times with DMEM F-12 media with 10% serum in a Petri dish. Each wash was performed in a new Petri dish.

All the MOs were incubated with 1 ml growth media with 10% serum, in 24 well/plate (SARSTEDT cat #80.1836.500 for Suspension Cells) at 5% CO₂ incubator 32° C. for 48 hrs.

Certain MO's were transduced with AAV-LK19-MAR-optEPO-WPRE 1.5*10¹³ vp/ml. The vector was diluted in growth media containing 10% DCS serum to final concentration of 1.5×10¹¹ vp/BP (10.0 μl/BP).

Other MO's were transduced with HdAd-EF1a-opt-hEPO 9.07*10¹² vp/ml. The vector was diluted in growth media containing 10% DSC serum to final concentration of 1.5×10¹⁰ vp/BP (1.66 μl/BP).

250 μl of transduction medium was added to each well using 1 ml pipettor. The plate was placed on a designated tray and incubated at 32° C., 5% CO₂, for 24 hours, with 150 rpm shaking for the first 4 hours.

GMMOs were washed from the transduction medium, and growth medium was added according to the open system SOP. Namely, the 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). This was repeated 3 times. The GMMOs were transferred to a new 24 well plate with fresh 1 ml growth media in each well. The plate was incubated at 32° C., 5% CO₂ for 3 days.

For the in vivo experiments, the control and test GMMOs/MOs were transferred from from the manufacturing facility to the clinic in incubator at 32° C., w/o CO₂ in 2 ml cryotube containing 2 ml growth medium (2.5 hrs transport). All GMMOs/MOs were washed in saline X6 washes prior to implantation.

In some instances, Depo-Medrol® (40 mg/ml, Pfizer) was injected as follows: 1 mg depomedrol/GMMO/MO (25 ul DepoMedrol® stock+75 ul saline/GMMO/MO). Mice were bled every 10 days and EPO in the serum was measured by ELISA.

The in-vitro hEPO levels secreted by each GMMO was measured by ELISA one day prior to implantation of each GMMO into the mice. In one experiment, two groups of five mice were implanted with two GMMOs transduced with HDAd-MAR-EF1α-opt-hEPO, one group after in-vitro processing of 3 days and the second group after processing of 9 days. The same process was followed in ten GMMOs transduced with AAV-MAR-CAG-opt-hEPO-WPRE vector. Two GMMOs per mouse were implanted subcutaneously on the dorsal side of the mouse, one on each side, using 10G implantation needles. Baseline bleed—mice were bled 6 days prior to GMMOs implantation in order to measure the background concentration of hEPO. Once implanted, blood samples were collected every 10-14 days for 17 weeks for the HDAd transduced GMMOs and for 35 weeks for the AAV transduced GMMOs in order to measure the in-vivo GMMOs performance. Serum hEPO levels in the blood were measured by a hEPO ELISA kit according to the protocol suggested by the manufacturer.

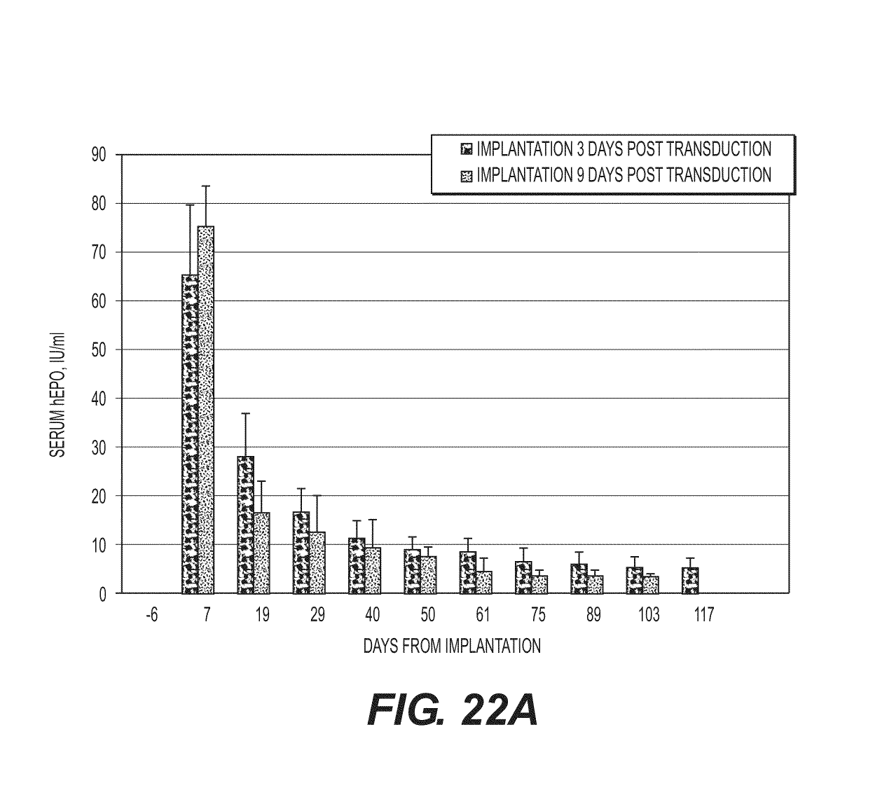

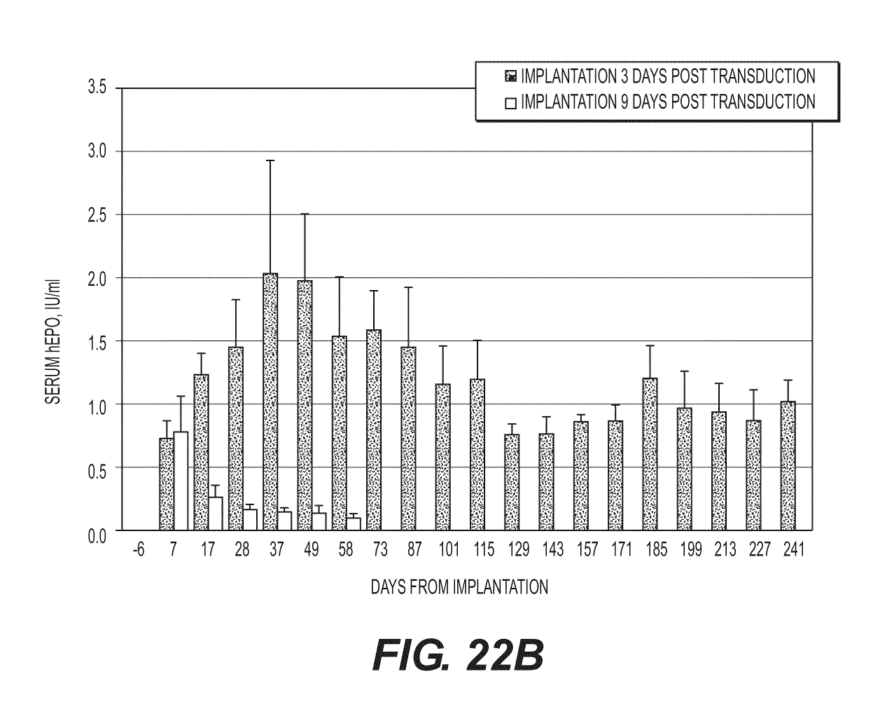

FIGS. 22a and 22b show the effect of the in-vitro processing period on the resulting GMMOs in-vivo performance when transduced with one of the two types of viral vectors, HDAd or AAV. FIG. 22a shows an improvement in the in-vivo performance of HDAd transduced GMMOs when the in-vitro GMMO processing duration was reduced from 9 to 3 days. FIG. 22b shows an improvement in the in-vivo performance of AAV transduced GMMOs when the in-vitro GMMO processing duration was reduced from 9 to 3 days.

On day 7 post implantation, similar secretion levels were obtained between the GMMOs processed in-vitro for 9 or 3 days. However, the protein levels in the serum dropped dramatically immediately after day 7 in mice implanted with the 9 day in-vitro processed GMMOs. At day 58 post implantation, a 14 fold difference was recorded in hEPO serum levels of SCID mice implanted with AAV transduced GMMOs processed in-vitro for 9 days as compared to mice implanted with an AAV transduced GMMO processed for 3 days. (See, e.g., FIG. 22b).

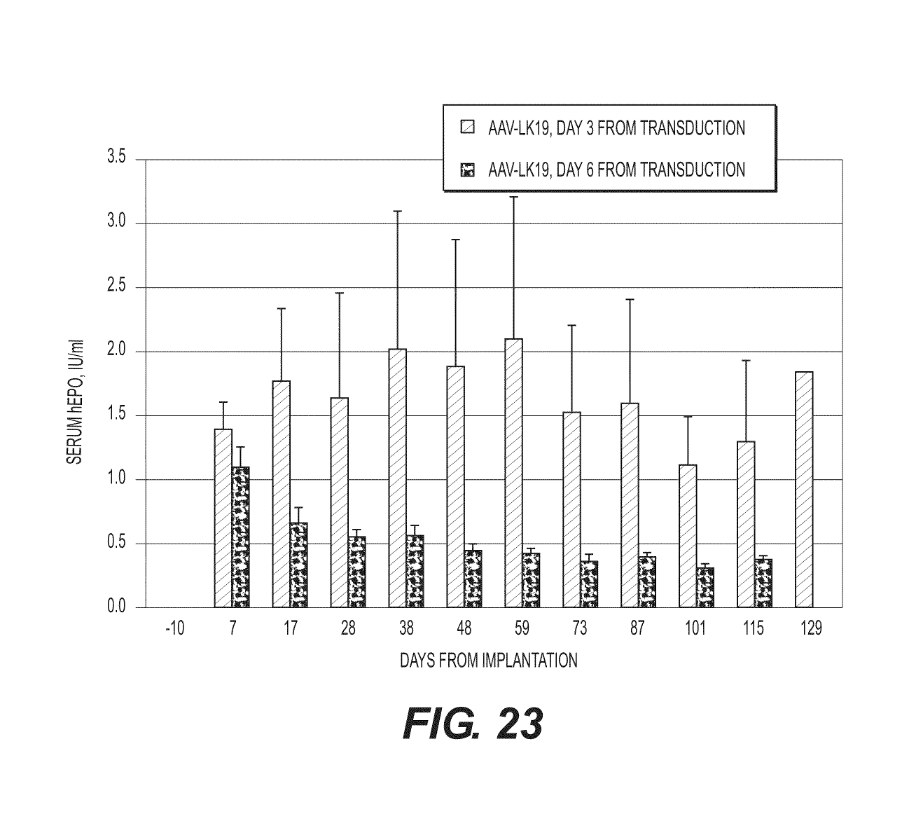

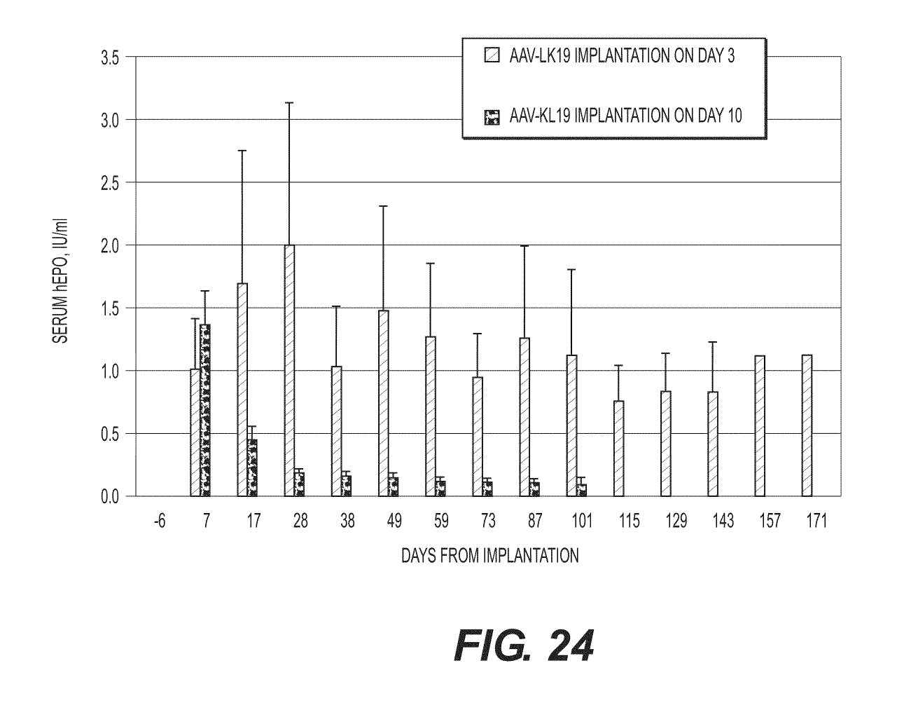

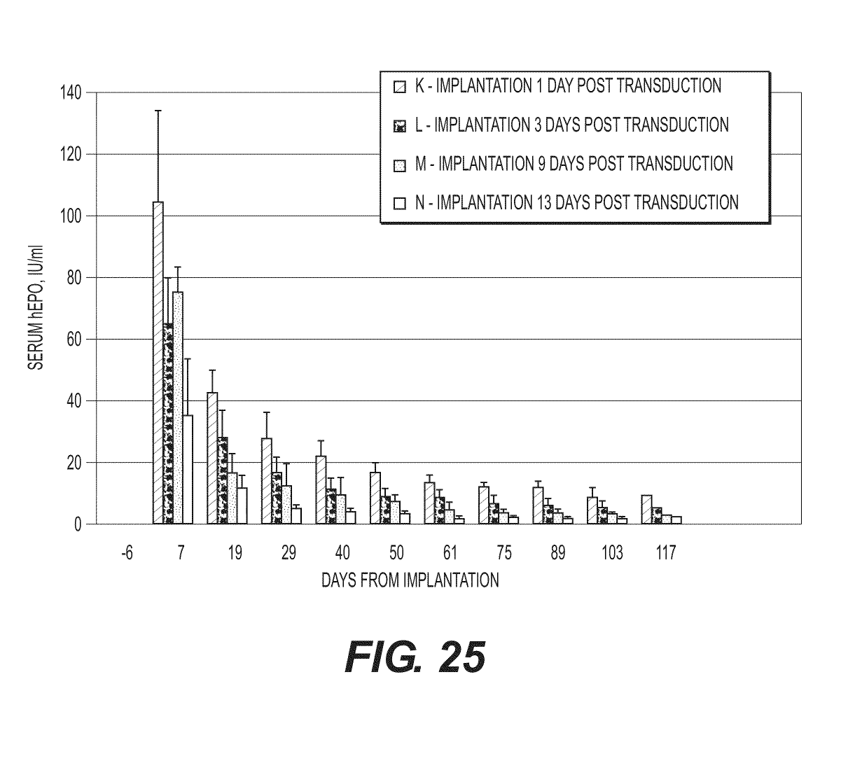

Similar results were obtained when the processing time was reduced from 13 days to 10 days, 9 days, 6 days, 3 days, and 1 day (See, e.g., FIGS. 23-25). Thus, reducing the in-vitro processing time from a standard 13 days or greater to less than 9 days-down to 1 day, significantly improves in vivo efficacy of the GMMO.

### Example 10

In this study, IFN GMMOs transduced with HDAd-CAG opt-IFNa and HDAd-EF1α-opt IFNa were assessed. In vitro processing times of 9 days, 4 days, and 2 days were compared. In particular, IFN GMMOs were implanted into SCID mice on different days post harvest (day 2, day 4, and day 9). DepoMedrol® was provided to the SCID mouse on implantation day and every two weeks thereafter.

### Materials and Methods:

Growth Media: DME/F-12 medium with 10% DCS (defined calf serum): HyClone DME/F-12 1:1 (xi)+2.50 mM L-Glutamine+15 mM HEPES Buffer (Thermo scientific, Cat# SH30023.01). Medium is supplemented with 10% DCS (HyClone Defined Bovine Calf Serum supplemented, Thermo scientific, Cat # SH30072.03); AmBisome 2.5 μg/ml (Liposomal Amphotericin B 50 mg—Gilead); Gentamycin sulfate 50 μg/ml (Gentamicin-IKA 80 mg/2 ml-Teva).

Viral vectors: pAd-CAG-optINFα (SEQ ID NO: 28) at a titer of 7.32×10e12 vp/ml. delta28-MAR-EF1α-optINFa (SEQ ID NO: 22) with a titer of 1.60×10e13 vp/ml.

### Experimental Procedure

Forty nine dermal core MOs approximately 30 mm were prepared in a sterile hood following the harvesting using a driller set at 7000 rpm and 14G needles (2.05 mm deep from skin surface) and back vacuum containing 2 ml of saline. The MO's were flushed out from the needles with saline. Needles were replaced every 4-5 harvest. The MO's were incubated for one minute in saline. All of the MO's were washed 3 times with DMEM F-12 media W/O serum in a Petri dish (all the MO's were cleaned). Every wash was performed in a new Petri dish.

All the MOs were incubated with 1 ml growth media in 24well/plate (SARSTEDT cat #80.1836.500 for Suspension Cells) at 5% CO₂ incubator 32° C. for 24 hrs.

### Viral Transduction:

Certain MO's were transduced with HDAd comprising the CAG-optINFa expression cassette (SEQ ID NO: 28) at 7.32*10¹² vp/ml. The HDAd vector was diluted in growth media containing 10% DCS serum to final concentration of 1.5×10¹⁰ vp/BP (2.05 μl/BP).

Certain MO's were transduced with HDAd comprising CpGfree-optINFa (SEQ ID NO: 23) 1.60*10′³ vp/ml. The vector was diluted in growth media containing 10% DCS serum to final concentration of 1.5×10¹⁰ vp/BP (0.94 μl/BP).

250 μl of transduction medium (growth media plus viral vector) was added to each well using 1 ml pipettor. The plate was placed on a designated tray and incubated at 32° C., 5% CO₂, for 24 hours, with 150 rpm shaking for the first 4 hours.

IFNa GMMOs were washed to remove the transduction medium, and growth medium was added. In general, the 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). 3 additional washes were done. The GMMOs were transferred to a new 24 well plate with fresh 1 ml growth media in each well. The plate was incubated at 32° C., 5% CO₂ for 3 days.

### Implantation

The IFN GMMOs were transferred from the manufacturing site to the clinic in incubator at 32° C., w/o CO₂ in 2 ml cryotube containing 2 ml growth medium (2.5 hrs transport). All GMMOs were washed 6 times in saline prior to implantation.

Two GMMOs were implanted SQ in each mouse with 10G needle.

Depo-Medrol® (40 mg/ml, Pfizer) was injected to certain test mice on implantation and every two weeks thereafter. The injections were as follows: 1 mg Depo-Medrol® per GMMO (25 micro liters Depo-Medrol® stock+75 micro liters saline/GMMO).

Mice were bled after one week, and then every 10 days and IFN in the serum was measured by ELISA.

### Results

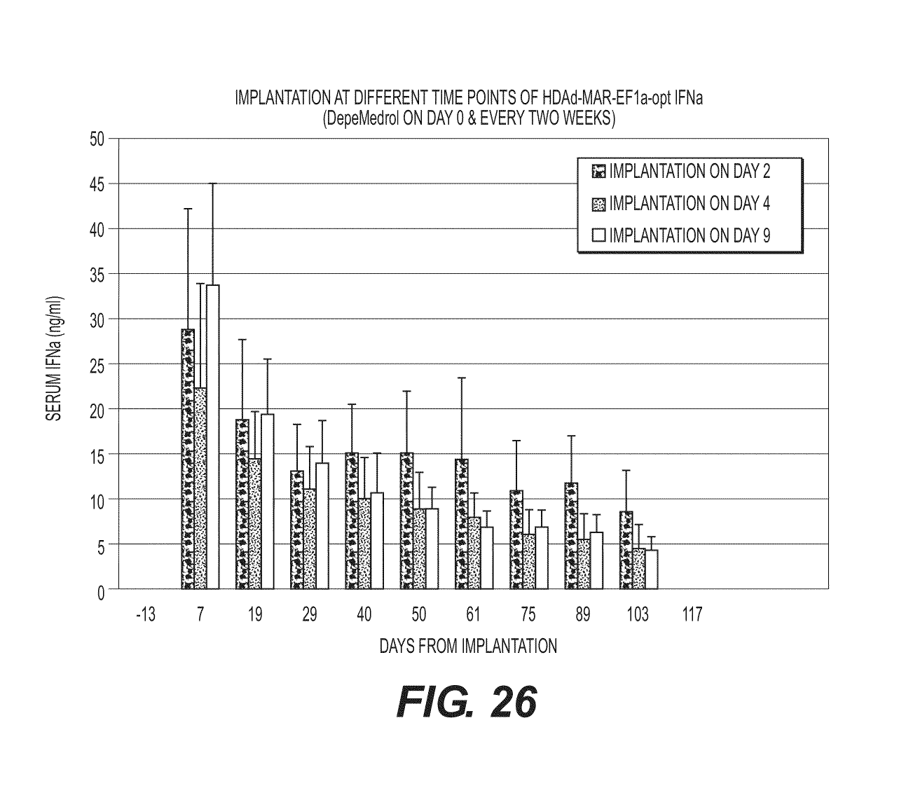

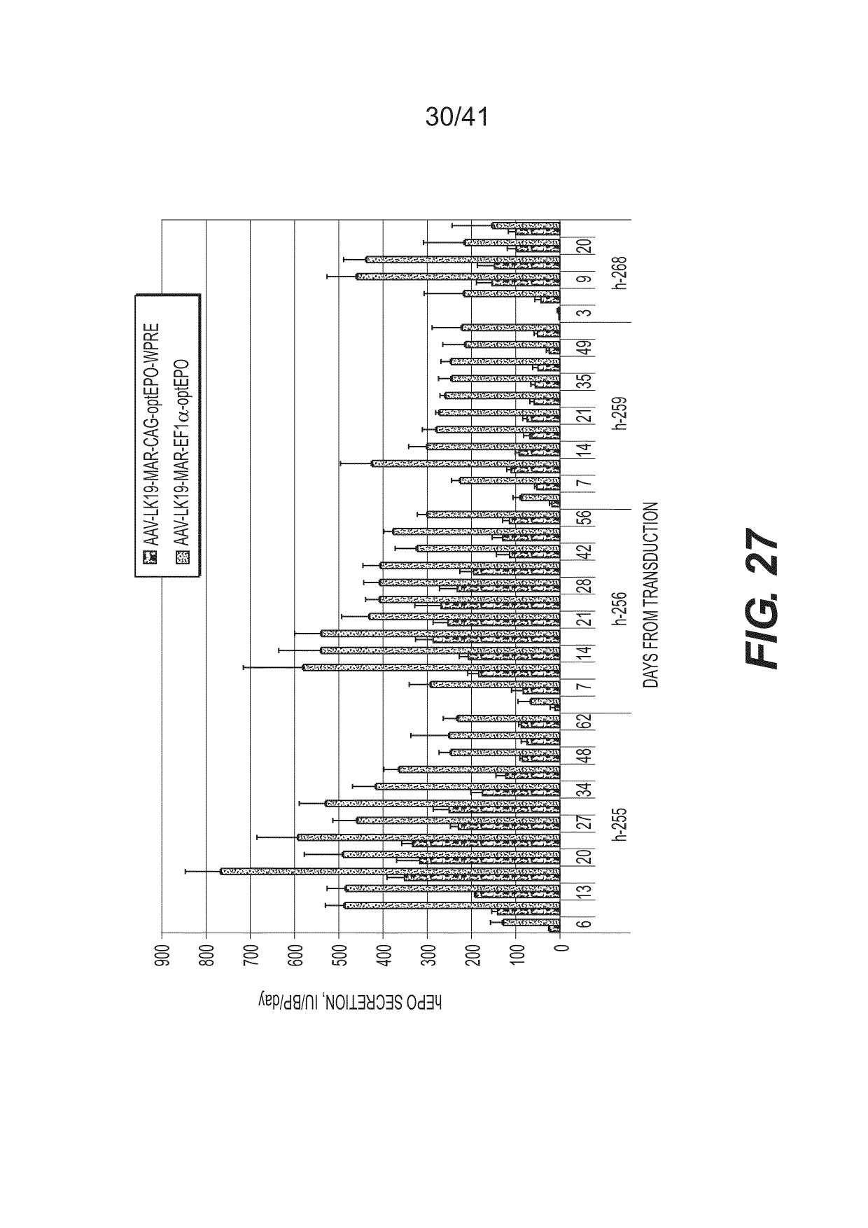

Results are depicted in FIG. 26, which suggests that a reduction in vitro processing time from nine to two days results in elevated serum INFa alpha levels at about 30-40 days and beyond.

### Example 11

### Overview

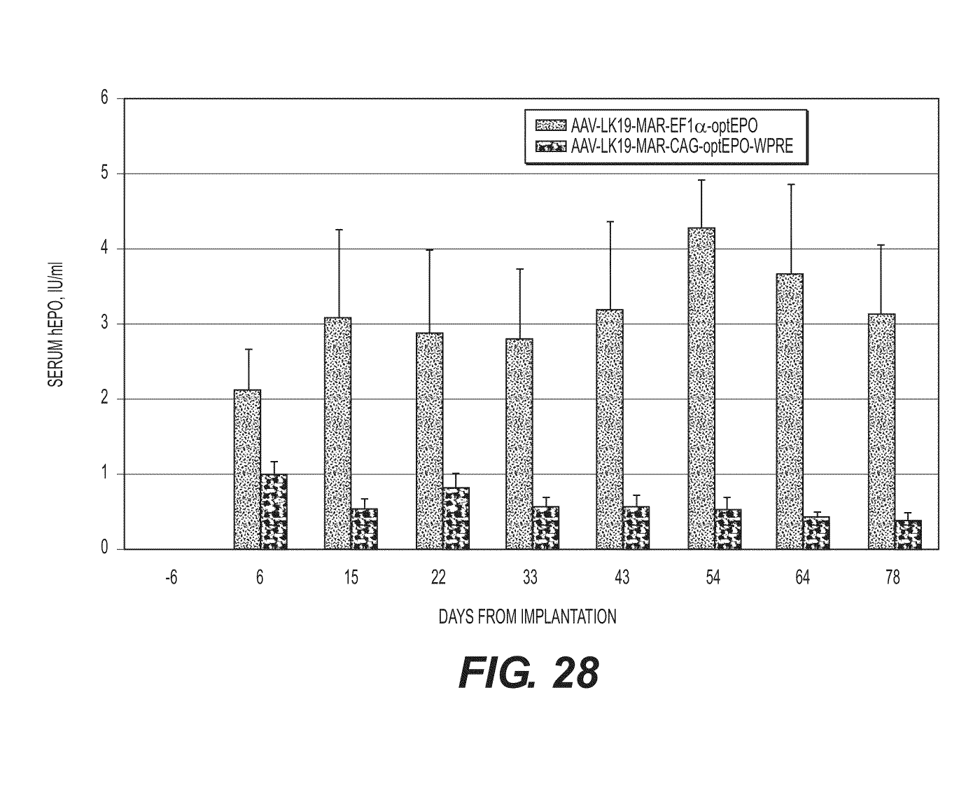

In the following study, the in vivo secretion profiles of EPO GMMOs transduced with AAV-LK19-MAR-EF1α-optEPO and AAV-LK19-MAR-CAG-optEPO-WPRE was tested. Both GMMOs produced hEPO in the serum after implantation. The MAR-EF1a-optEPO vector produced elevated levels as compared to MAR-CAG-optEPO-WPRE. See, FIG. 28.

Materials and Methods:

DME/F-12 medium with 10% DCS (defined calf serum): HyClone DME/F-12 1:1 (X1)+2.50 mM L-Glutamine+15 mM HEPES Buffer (Thermo scientific, Cat# SH30023.01). Medium is supplemented with 10% DCS (HyClone Defined Bovine Calf Serum supplemented, Thermo scientific, Cat # SH30072.03); AmBisome 2.5 μg/ml (Liposomal Amphotericin B 50 mg—Gilead); Gentamycin sulfate 50 μg/ml (Gentamicin-IKA 80 mg/2 ml—Teva).

Viral transduction: Certain MOs were transduced with AAV-LK19-EF1a-optEPO Lot 1, 1.2*10¹³ vp/BP. The vector was diluted in Growth media containing 10% DCS serum to final concentration of 1.5×10¹¹ vp/MO (12.5 μl/MO). Other MOs were transduced with AAV-LK19-MAR-optEPO-WPRE Lot 3, 1.0*10¹³ vp/BP. The vector was diluted in Growth media containing 10% DCS serum to final concentration of 1.5×10¹¹ vp/BP (15 μl/BP).

The GMMO's/MO's were transferred to the clinic in an incubator at 32° C., w/o CO₂ in 2 ml cryotube containing 2 ml growth medium (2.5 hrs transport). All GMMO's/MO's were washed in saline X6 washes prior to implantation. 2 GMMOs were implanted SQ in each mouse with an implantation device with 10G needle. Depo-Medrol® (40 mg/ml, Pfizer) was injected to all groups on implantation and day 45 from implantation. The injections were as following: 1 mg Depo-Medrol®/GMMO&MO (25 μl Depo-Medrol® stock+75 μl saline/GMMO&MO).

### Example 12

### Overview

The in vitro performance of GMMOs transduced with different AAV viral vectors was assessed. Each of the AAV viral vectors comprising the EPO expression cassettes described herein were capable of secreting hEPO in vitro.

### Results

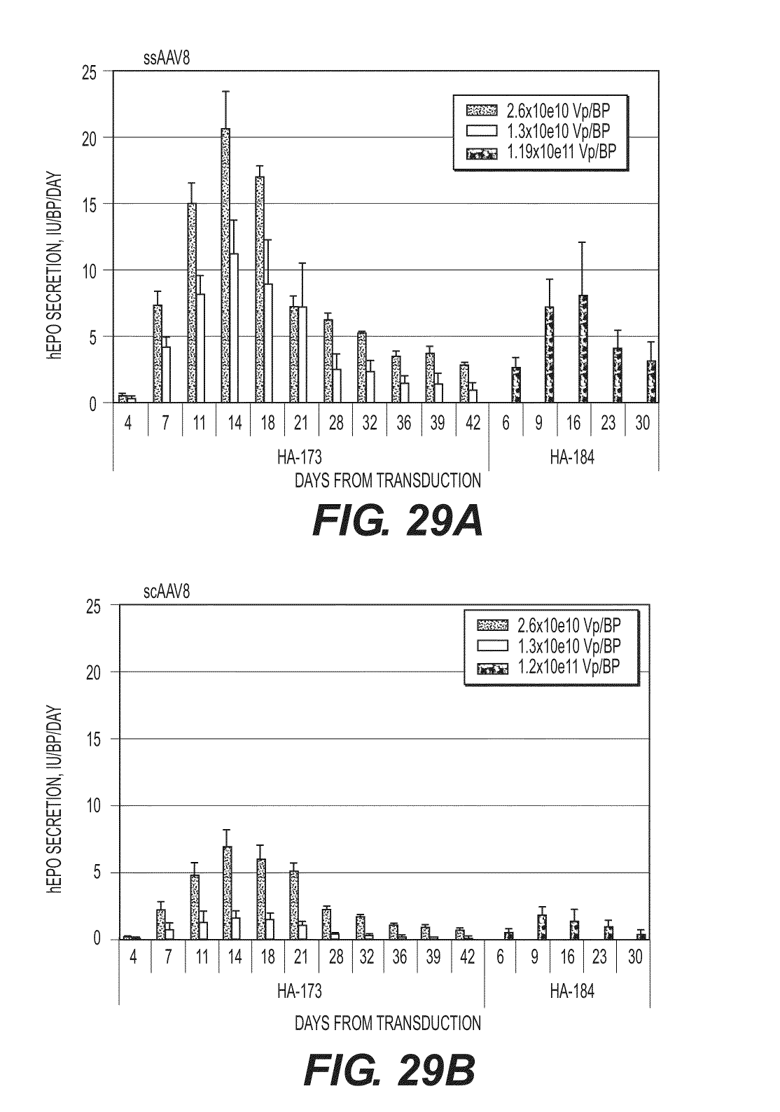

FIGS. 29a and 29b shows in vitro performance of two different types of AAV vectors comprising ssAAV8-MAR-CAG-optEPO-WPRE and scAAV8-MAR-CAG-optEPO-WPRE. Both AAV vectors were able to generate EPO GMMOs that were capable of secreting hEPO in vitro for at least 42 days.

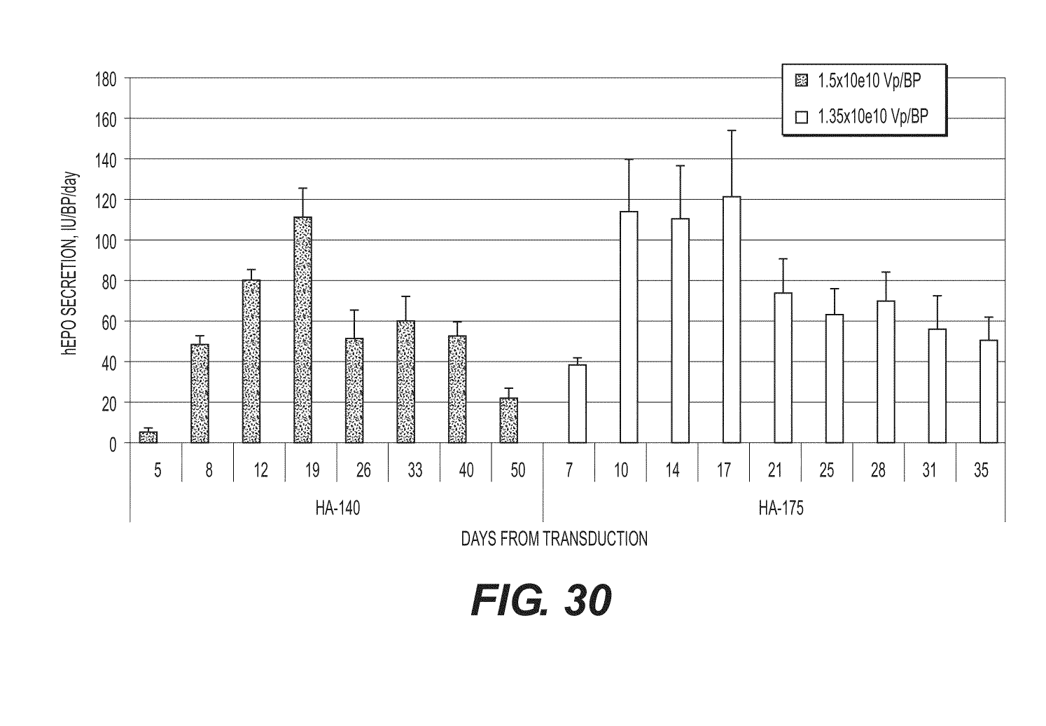

FIG. 30 shows in vitro performance of a different AAV vector, AAV1/2, comprising AAV1/2-MAR-CAG-wtEPO. AAV1/2-MAR-CAG-wtEPO was able to generate EPO GMMOs that were capable of secreting hEPO in vitro for at least 50 days.

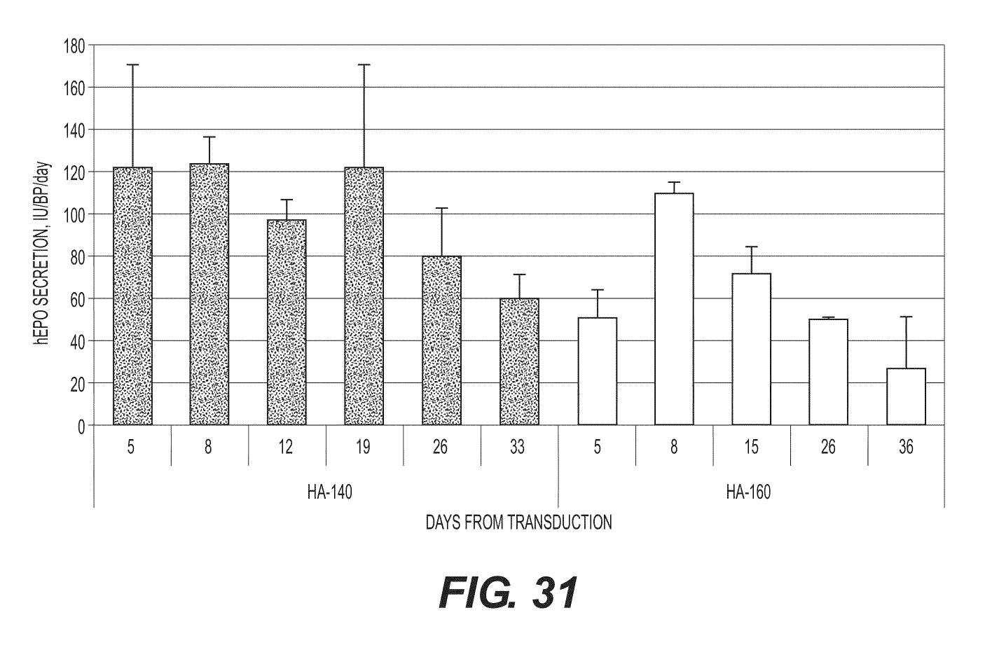

FIG. 31 shows in vitro performance of a different AAV vector, AAV1, comprising scAAV2/1-CAG-wtEPO. scAAV2/1-CAG-wtEPO was able to generate EPO GMMOs that were capable of secreting hEPO in vitro for at least 33 days.

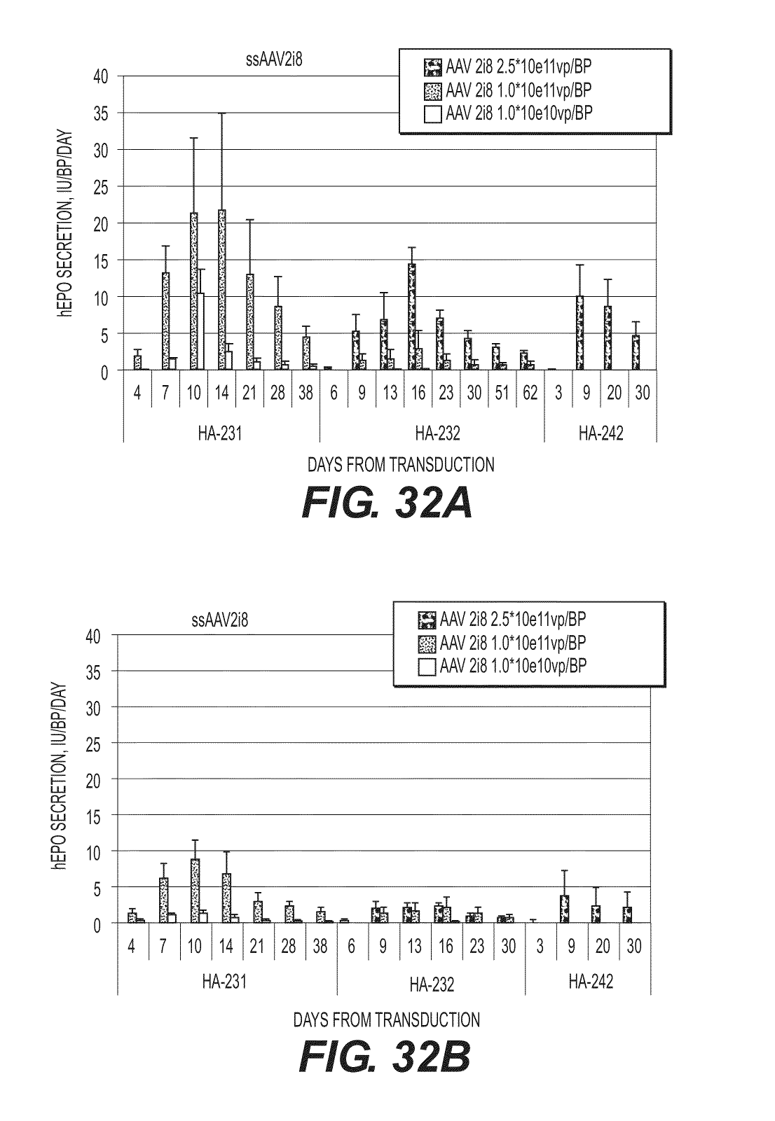

FIGS. 32a and 32b show in vitro performance of two different AAV vectors, ssAAV2i8 and scAAV2i8, comprising ssAAV2i8-MAR-CAG-optEPO-WPRE and sc AAV2i8-CAG-optEPO. hEPO was secreted in vitro from EPO GMMOs comprising ssAAV2i8-MAR-CAG-optEPO-WPRE and sc AAV2i8-CAG-optEPO for at least 62 days.

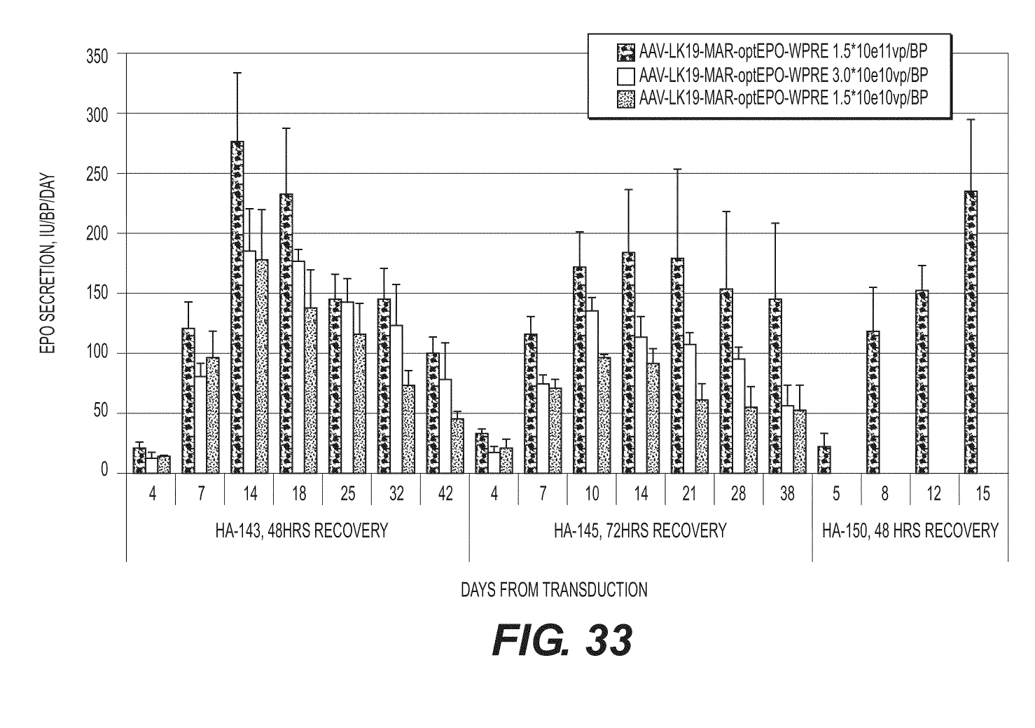

FIG. 33 shows in vitro performance of a different AAV vector, AAV-LK19, comprising MAR-CAG-optEPO-WPRE expression cassettes. hEPO was secreted in vitro from EPO GMMOs comprising AAV-LK19-MAR-optEPO-WPRE for at least 42 days.

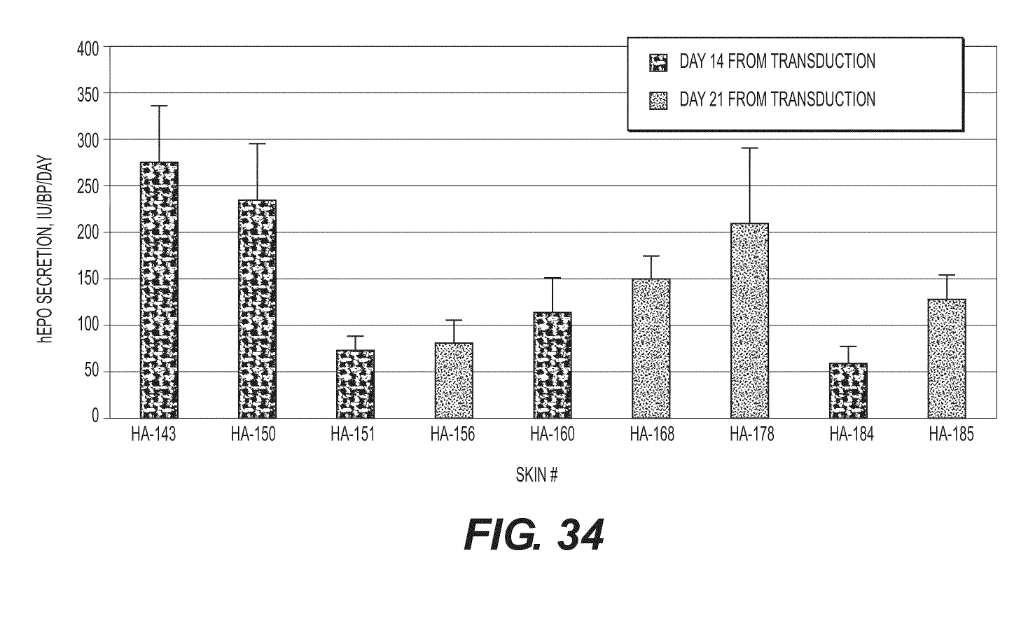

FIG. 34 shows in vitro skin to skin performance variability of EPO GMMO comprising AAV-LK19-MAR-CAG-optEPO-WPRE. Different donor MOs are indicated by “HA-number”. AAV-LK19-MAR-CAG-optEPO-WPRE comprising GMMOs secreted EPO in each skin type tested.

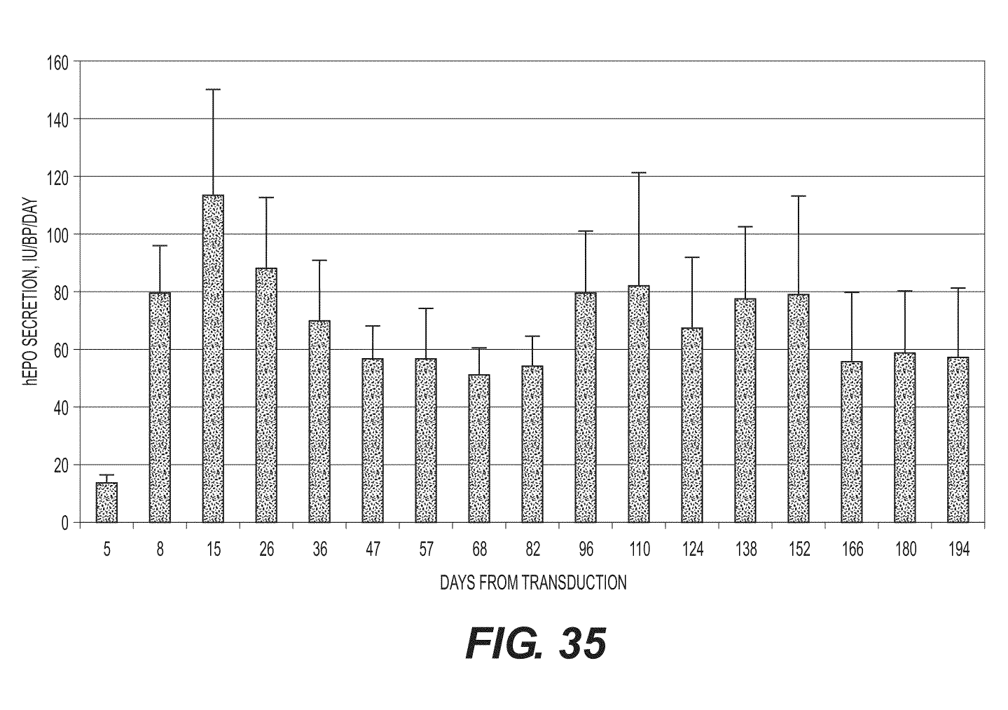

FIG. 35 shows the long term in vitro secretion profile of an EPO GMMO comprising AAV-LK19-MAR-CAG-optEPO-WPRE. Relatively steady hEPO was observed for more than 6 months.

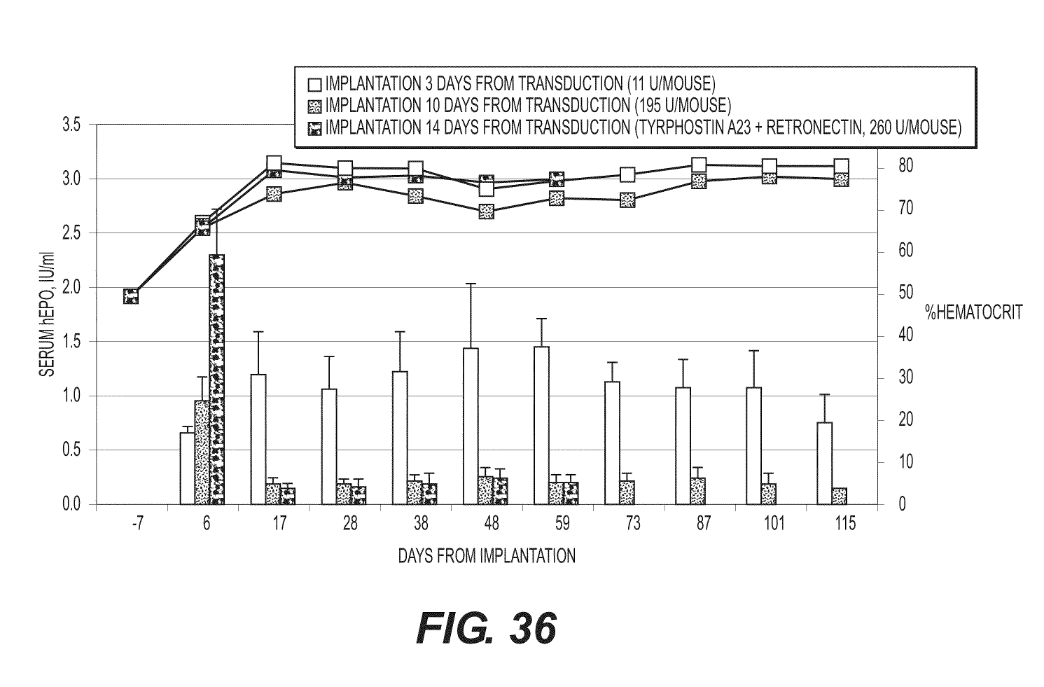

FIG. 36 shows the effect of in vitro processing time on the GMMOs in vivo performance. AAV-LK19-MAR-CAG-optEPO-WPRE was used to transduce MOs and the transduced MOs were maintained in vitro for 3, 10, or 14 days prior to implantation. As seen with HDAd transduced MOs, AAV transduced MOs also secreted higher levels of hEPO and provided an increased hematocrit when the in vitro processing time was reduced from 14 to 10 to 3 days.

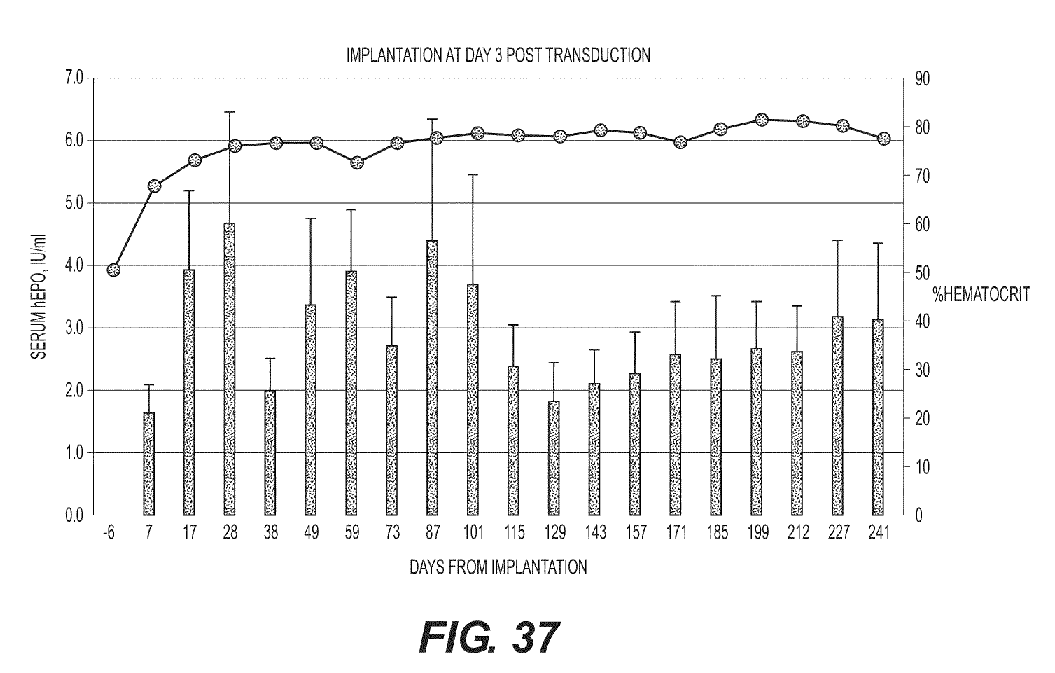

FIG. 37 shows the long term in vivo secretion profile of EPO GMMOs comprising AAV-LK19-MAR-CAG-optEPO-WPRE. Two EPO GMMOs comprising AAV-LK19-MAR-CAG-optEPO-WPRE were implanted into SCID mice and serum hEPO and hematocrit were assessed. The results show at least 241 days of steady hEPO secretion.

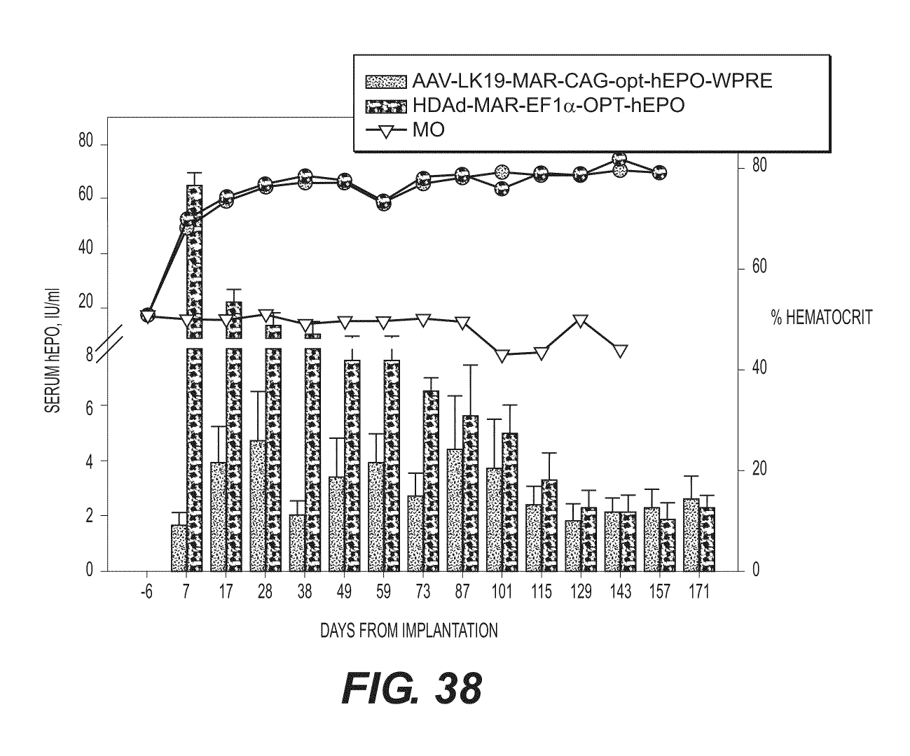

FIG. 38 shows the in vivo performance of EPO GMMOs comprising HDAd-MAR-EF1a-opt-hEPO compared to AAV-LK19-MAR-CAG-opt-hEPO-WPRE. While the HDAd transduced GMMOs initially had higher in vivo secretion levels than the AAV transduced GMMOs, by about 3 months the measured levels of EPO in the serum was about the same. When observed for longer periods of time, the AAV transduced GMMOs maintained EPO levels in the serum while the level of EPO in the serum of mice transduced with HDAd GMMOs declined (data not shown).

### CONCLUSIONS

HDAd and AAV vectors are efficient carriers to transduce micro-organs to produce genetically modified micro-organs that express therapeutic proteins that provide sustained delivery of therapeutic polypeptides. Efficacy has been demonstrated in vitro, in vivo in animal models, and in human clinical trials.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A genetically modified micro-organ that provides a sustained delivery of a therapeutic polypeptide, said micro-organ comprising a vector comprising a nucleic acid sequence encoding said therapeutic polypeptide operably linked to an upstream MAR regulatory sequence and comprising at least one additional regulatory sequence, wherein said at least one genetically modified micro-organ expresses said therapeutic polypeptide for a sustained period of at least three months in a subject in vivo.

2. The genetically modified micro-organ of claim 1, wherein said therapeutic polypeptide is human erythropoietin or human interferon.

3. The genetically modified micro-organ of claim 2, wherein said therapeutic polypeptide is human interferon and said human interferon is an interferon α, an interferon β, an interferon λ, or an interferon γ.

4. The genetically modified micro-organ of claim 1, wherein said at least one additional regulatory sequence comprises a MAR sequence, a CAG promoter sequence, an EF1α promoter sequence or a WPRE sequence.

5. The genetically modified micro-organ of claim 1, wherein said nucleic acid sequence comprises SEQ ID NO: 11, SEQ ID NO: 13, SEQ ID NO: 15, SEQ ID NO: 17, SEQ ID NO: 23, or SEQ ID NO: 25, or a nucleic acid sequence at least 95% identical to SEQ ID NO: 11, SEQ ID NO: 13, SEQ ID NO: 15, SEQ ID NO: 17, SEQ ID NO: 23, or SEQ ID NO: 25.

6. The genetically modified micro-organ of claim 1, wherein said vector is a helper dependent adenovirus (HdAd) vector or an adeno-associated virus (AAV) vector.

7. The genetically modified micro-organ of claim 1, wherein said genetically modified micro-organ is a genetically modified dermal micro-organ.

8. A method of treating anemia in a human subject in need thereof over a sustained time period comprising the steps of:
providing at least one genetically modified micro-organ according to claim 2 that provides a sustained delivery of a human erythropoietin;
determining erythropoietin secretion levels of said at least one genetically modified micro-organ in vitro;
implanting said at least one genetically modified micro-organ in said human subject at an effective dosage; and
measuring erythropoietin levels in the blood serum of said subject;
wherein implantation of said at least one genetically modified micro-organ increases the in vivo serum erythropoietin levels over basal levels for at least three months.

9-13. (canceled)

14. The method of claim 8, further comprising a step of administering methylprednisolone following said implanting step, wherein said administering is by subcutaneous injection around each genetically modified micro-organ implantation site, and optionally repeating the administration every 2 weeks post-implantation for a total of 8 weeks of methylprednisolone treatment.

15-27. (canceled)

28. The method of claim 8, wherein the implanted at least one genetically modified micro-organ provides continuously secreted erythropoietin for at least three months in said subject.

29. The method of claim 8, wherein said in vivo serum erythropoietin levels are increased over basal levels for at least six months, or wherein the in vivo serum erythropoietin levels have decreased decay rates over basal levels for at least six months, or wherein the genetically modified micro-organ maintains percent hematocrit over basal levels for at least six months, or wherein the genetically modified micro-organ is capable of autoregulating hemoglobin levels.

30-32. (canceled)

33. The method of claim 8, further comprising maintaining said at least one genetically modified micro-organ in vitro for less than 9 days prior to said implanting.

34-36. (canceled)

37. A method of providing increased serum erythropoietin levels in a human subject over a sustained period of time comprising the steps of:
providing at least one genetically modified micro-organ according to claim 2 that provides a sustained delivery of a human erythropoietin;
determining erythropoietin secretion levels of said at least one genetically modified micro-organ in vitro;
implanting said at least one genetically modified micro-organ in said subject at an effective dosage; and
measuring erythropoietin levels in the blood serum of said subject,
wherein implantation of said at least one genetically modified micro-organ increases the in vivo serum erythropoietin levels over basal levels for at least three months.

38-41. (canceled)

42. The method of claim 37, further comprising a step of administering methylprednisolone following said implanting step, wherein said administering is by subcutaneous injection around each genetically modified micro-organ implantation site, and optionally repeating the methylprednisolone administration every 2 weeks for a total of 8 weeks.

43-58. (canceled)

59. The method of claim 37, further comprising a step of implanting at a later date to said subject, at least one additional genetically modified micro-organ that provides a sustained delivery of a human erythropoietin, wherein the at least one additional genetically modified micro-organ comprises a vector comprising a nucleic acid sequence encoding said human erythropoietin operably linked to an upstream MAR regulatory sequence and comprising at least one additional regulatory sequence, wherein said at least one additional genetically modified micro-organ expresses said human erythropoietin for a sustained period of at least three months in a subject in vivo.

60. The method of claim 59, further comprising a step of administering methylprednisolone following said implanting of said at least one additional genetically modified micro-organ, wherein said administering is by subcutaneous injection around each genetically modified micro-organ implantation site.

61. The method of claim 37, further comprising a step of maintaining said at least one genetically modified micro-organ in vitro for less than 9 days prior to said implanting.

62-66. (canceled)

67. A method of providing a therapeutic polypeptide to a subject in need thereof over a sustained time period, said method comprising the steps of:
providing at least one genetically modified micro-organ expressing and secreting a therapeutic polypeptide determining in vitro secretion levels of said therapeutic polypeptide from said at least one genetically modified micro-organ;
implanting said at least one genetically modified micro-organ in a subject; and
administering methylprednisolone by subcutaneous injection around each genetically modified micro-organ implantation site following said implanting step;
wherein said method provides said therapeutic polypeptide to said subject for a sustained time period of at least three months.

68. The method of claim 67, wherein said at least one genetically modified micro-organ is a genetically modified dermal micro-organ comprising an incomplete epidermal layer.

69-76. (canceled)

77. A method of treating hepatitis in a human subject in need thereof over a sustained time period comprising the steps of:
providing at least one genetically modified micro-organ according to claim 2 that provides a sustained delivery of a human interferon;
determining interferon secretion levels of said at least one genetically modified micro-organ in vitro;
implanting said at least one genetically modified micro-organ in said human subject at an effective dosage; and
measuring interferon levels in the blood serum of said subject;
wherein implantation of said at least one genetically modified micro-organ increases the in vivo serum interferon levels over basal levels for at least three months.

78-81. (canceled)

82. The method of claim 77, further comprising a step of administering methylprednisolone following said implanting step, wherein said administering is by subcutaneous injection around each genetically modified micro-organ implantation site, and optionally repeating the methylprednisolone administration every 2 weeks for a total of 8 weeks.

83-88. (canceled)

89. The method of claim 77, further comprising a step of implanting at a later date to said subject, at least one additional genetically modified micro-organ that provides a sustained delivery of a human interferon, wherein said at least one additional micro-organ comprises a vector comprising a nucleic acid sequence encoding said human interferon operably linked to an upstream MAR regulatory sequence and comprising at least one additional regulatory sequence, wherein said at least one genetically modified micro-organ expresses said human interferon for a sustained period of at least three months in a subject in vivo.

90-92. (canceled)

93. A method of providing increased serum interferon levels in a human subject over a sustained period of time comprising the steps of:
providing at least one genetically modified micro-organ according to claim 2 that provides a sustained delivery of a human interferon;
determining interferon secretion levels of said at least one genetically modified micro-organ in vitro;
implanting said at least one genetically modified micro-organ in said subject at an effective dosage; and
measuring interferon levels in the blood serum of said subject,
wherein implantation of said at least one genetically modified micro-organ increases the in vivo serum interferon levels over basal levels for at least three months.

94-97. (canceled)

98. The method of claim 93, further comprising a step of administering methylprednisolone following said implanting step, wherein said administering is by subcutaneous injection around each genetically modified micro-organ implantation site, and optionally repeating the methylprednisolone administration every 2 weeks for a total of 8 weeks.

94-103. (canceled)

104. The method of claim 93, further comprising a step of implanting at a later date to said subject, at least one additional genetically modified micro-organ that provides a sustained delivery of a human interferon, wherein said at least one additional micro-organ comprises a vector comprising a nucleic acid sequence encoding said human interferon operably linked to an upstream MAR regulatory sequence and comprising at least one additional regulatory sequence, wherein said at least one genetically modified micro-organ expresses said human interferon for a sustained period of at least three months in a subject in vivo.

105. (canceled)

106. The method of claim 93, further comprising a step of maintaining said at least one genetically modified micro-organ in vitro for less than 9 days prior to said implantation step.

107-110. (canceled)

111. A genetically modified dermal micro-organ comprising the nucleic acids of SEQ ID NO: 10, SEQ ID NO: 11, SEQ ID NO: 22, SEQ ID NO: 23, or SEQ ID NO: 27, or nucleic acids that are at least 95% identical to SEQ ID NO: 10, SEQ ID NO: 11, SEQ ID NO: 22, SEQ ID NO: 23, or SEQ ID NO: 27.

112. The at least one genetically modified micro-organ that provides a sustained delivery of a therapeutic polypeptide of claim 1, wherein the genetically modified micro-organ expresses said therapeutic polypeptide for at least six months in a subject in vivo.

113. The at least one genetically modified micro-organ that provides a sustained delivery of a therapeutic polypeptide of claim 2, wherein the genetically modified micro-organ expresses said therapeutic polypeptide for at least six months in a subject in vivo.


SEQUENCE LISTING (28 sequences)

SEQ ID NO: 1 - DNA, 582 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 2 - DNA, 582 bp, Artificial Sequence
  Description of Artificial Sequence Synthetic optimized EPO polynucleotide
atgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctga

SEQ ID NO: 3 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 4 - DNA, 810 bp, Artificial Sequence
  Description of Artificial Sequence Synthetic IFNbeta S/MAR polynucleotide
actgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagtt

SEQ ID NO: 5 - DNA, 808 bp, Artificial Sequence
  Description of Artificial Sequence Synthetic 5′ region of IFNbeta S/MAR polynucleotide
agtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaa

SEQ ID NO: 6 - DNA, 427 bp, Artificial Sequence
  Description of Artificial Sequence Synthetic CpG free beta-globin MAR polynucleotide
ttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcag

SEQ ID NO: 7 - DNA, 1177 bp, Artificial Sequence
  Description of Artificial Sequence Synthetic CAG promoter polynucleotide
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagt

SEQ ID NO: 8 - DNA, 590 bp, Artificial Sequence
  Description of Artificial Sequence Synthetic WPRE polynucleotide
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 9 - DNA, 230 bp, Artificial Sequence
  Description of Artificial Sequence Synthetic polynucleotide
ggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatc

SEQ ID NO: 10 - DNA, 33057 bp, Artificial Sequence
  Description of Artificial Sequence Synthetic pdelta28-MAR-EF1alpha-optEPO polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttaaggatctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatccccatccagcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 11 - DNA, 2914 bp, Artificial Sequence
  Description of Artificial Sequence Synthetic MAR-EF1alpha-opt-hEPO Expression Cassette polynucleotide
ttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttaaggatctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatccccatccagcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaa

SEQ ID NO: 12 - DNA, 33638 bp, Artificial Sequence
  Description of Artificial Sequence Synthetic pdelta28-MAR-CAG-opt-hEPO-WPRE polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccctcgagctagcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttgctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatcccgggtagtcagcttgagtctgatatcaagcttattgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcattgatacccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 13 - DNA, 3484 bp, Artificial Sequence
  Description of Artificial Sequence Synthetic S/MAR-CAG-opt-hEPO-WPRE cassette polynucleotide
actgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttgctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatcccgggtagtcagcttgagtctgatatcaagcttattgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcattgatacccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatc

SEQ ID NO: 14 - DNA, 32187 bp, Artificial Sequence
  Description of Artificial Sequence Synthetic pdelta--CAG-wt-hEPO polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccctcgagctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 15 - DNA, 2027 bp, Artificial Sequence
  Description of Artificial Sequence Synthetic CAG-opt-hEPO cassette polynucleotide
ccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatc

SEQ ID NO: 16 - DNA, 32187 bp, Artificial Sequence
  Description of Artificial Sequence Synthetic pdelta--CAG-opt-hEPO polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccctcgagctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 17 - DNA, 2038 bp, Artificial Sequence
  Description of Artificial Sequence Synthetic CAG-wt-hEPO cassette polynucleotide
ctcgagctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatc

SEQ ID NO: 18 - DNA, 224 bp, Artificial Sequence
  Description of Artificial Sequence Synthetic EF1alpha promoter polynucleotide
actagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattc

SEQ ID NO: 19 - DNA, 567 bp, Homo sapiens
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 20 - DNA, 567 bp, Artificial Sequence
  Description of Artificial Sequence Synthetic optimized IFN polynucleotide
atggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtga

SEQ ID NO: 21 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 22 - DNA, 33064 bp, Artificial Sequence
  Description of Artificial Sequence Synthetic pdelta28-MAR-EF1alpha-optIFNalpha polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggccctgaccttcgctctgctggtggccctgctggtgctgagctgcaagagcagctgtagcgtggggtgcgacctgccccagacacacagcctgggcagccggcgcacactgatgctgctggcccagatgcggcgcatctccctgttcagctgcctgaaggaccgccacgacttcggctttccccaagaggagttcggcaaccagttccagaaggctgagaccatccccgtgctgcacgagatgatccagcagatcttcaacctgttcagcaccaaggacagcagcgccgcctgggatgagaccctgctggacaagttctacaccgagctgtaccagcagctgaacgacctggaggcctgcgtgattcagggggtgggggtcaccgagacccccctgatgaaggaggacagcatcctggccgtccgcaaatacttccagcgcatcaccctgtacctgaaggagaagaagtactccccctgcgcctgggaggtggtgcgcgccgagatcatgcggagcttcagcctgtccaccaacctccaggagagcctgcgctccaaggagtgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 23 - DNA, 2937 bp, Artificial Sequence
  Description of Artificial Sequence Synthetic MAR-EF1alpha-optIFNalpha expression cassette polynucleotide
ggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggccctgaccttcgctctgctggtggccctgctggtgctgagctgcaagagcagctgtagcgtggggtgcgacctgccccagacacacagcctgggcagccggcgcacactgatgctgctggcccagatgcggcgcatctccctgttcagctgcctgaaggaccgccacgacttcggctttccccaagaggagttcggcaaccagttccagaaggctgagaccatccccgtgctgcacgagatgatccagcagatcttcaacctgttcagcaccaaggacagcagcgccgcctgggatgagaccctgctggacaagttctacaccgagctgtaccagcagctgaacgacctggaggcctgcgtgattcagggggtgggggtcaccgagacccccctgatgaaggaggacagcatcctggccgtccgcaaatacttccagcgcatcaccctgtacctgaaggagaagaagtactccccctgcgcctgggaggtggtgcgcgccgagatcatgcggagcttcagcctgtccaccaacctccaggagagcctgcgctccaaggagtgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcc

SEQ ID NO: 24 - DNA, 33616 bp, Artificial Sequence
  Description of Artificial Sequence Synthetic pdelta28-MAR-CAG-optIFNalpha-WPRE polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccctcgagctagcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttgctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtgaggatccccgggtagtcagctgtagctgatatcaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcattgatacccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcgacgcggacgcgcgtaatacgactcactatagggcgaattggagctccactacgtagtttaaa

SEQ ID NO: 25 - DNA, 3462 bp, Artificial Sequence
  Description of Artificial Sequence Synthetic MAR-CAG-optIFNalpha-WPRE expression cassette polynucleotide
actgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttgctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtgaggatccccgggtagtcagctgtagctgatatcaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcattgatacccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatc

SEQ ID NO: 26 - DNA, 7189 bp, Artificial Sequence
  Description of Artificial Sequence Synthetic pAAV-LK19-MAR-CAG-opt-hEPO-WPRE polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtaacctcgagctagcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttgctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatcccgggtagtcagcttgagtctgatatcaagcttattgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcattgatacccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcttacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaagcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttarcagccggacgtgcaractccgctggtggtctaacgtcggttaggtcccttgaatcacgggacatatgttggtgttggaggt

SEQ ID NO: 27 - DNA, 6630 bp, Artificial Sequence
  Description of Artificial Sequence Synthetic pAAV-LK19-MAR-EF1alpha-opt-hEPO polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtaacttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgggcgtgcacgagtgccccgcctggctgtggctgctgctgtccctgctgtctctgcccctgggcctgcctgtgctgggagcccctccccggctgatctgcgacagccgggtgctggaaagatacctgctggaagccaaagaggccgagaacatcaccaccggctgcgccgagcactgcagcctgaacgagaatatcaccgtgcccgacaccaaggtgaacttctacgcctggaagcggatggaagtgggccagcaggccgtggaagtgtggcagggcctggccctgctgtccgaggccgtgctgagagggcaggccctgctggtgaacagcagccagccctgggagcctctgcagctgcacgtggacaaggccgtgagcggcctgcggagcctgaccaccctgctgagggccctgggcgcccagaaagaggccatcagcccccctgatgccgcctctgccgcccctctgcggaccatcaccgccgacaccttccggaagctgttccgggtgtacagcaacttcctgcggggcaagctgaagctgtacaccggcgaggcctgccggaccggcgatcgctgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaattacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaagcgcgcagctgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgcttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggcagctgcgcgctcgaacttcatgcctgccgaccttccccaggtcacgatccggacggcgggtgagttcacattttarcagccggacgtgcaractccgctggtggtctaacgtcggttaggtcccttgaatcacgggacatatgttggtgttggaggt

SEQ ID NO: 28 - DNA, 5246 bp, Artificial Sequence
  Description of Artificial Sequence Synthetic pAd-CAG-Opt INFa polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccctcgagctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgccgggcccctacgtcacccgccccgttcccacgccccgcgccacgtcacaaactccaccccctcattatcatattggcttcaatccaaaataaggtatattattgatgatggccgcagcggcccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggc